SEQUENCE LISTING (515 sequences)

SEQ ID NO: 1 - DNA, 22 bp, synthetic construct
  source 1..22: mol_type = other DNA
cttgtacctggtgttcggttat

SEQ ID NO: 2 - DNA, 20 bp, synthetic construct
  source 1..20: mol_type = other DNA
gtaggctgggtagccaaatc

SEQ ID NO: 3 - DNA, 20 bp, synthetic construct
  source 1..20: mol_type = other DNA
ccagcccaagatggatgaat

SEQ ID NO: 4 - RNA, 23 nt, synthetic construct
  source 1..23: mol_type = other RNA
ctcttctttcctttcagcaacac

SEQ ID NO: 5 - DNA, 21 bp, synthetic construct
  source 1..21: mol_type = other DNA
gcggtggaagtcattctgata

SEQ ID NO: 6 - DNA, 22 bp, synthetic construct
  source 1..22: mol_type = other DNA
tctctctctctccagtgaacaa

SEQ ID NO: 7 - DNA, 20 bp, synthetic construct
  source 1..20: mol_type = other DNA
gagggccacagattggtatt

SEQ ID NO: 8 - DNA, 24 bp, synthetic construct
  source 1..24: mol_type = other DNA
gatcacttagctcaggaaacaaac

SEQ ID NO: 9 - DNA, 22 bp, synthetic construct
  source 1..22: mol_type = other DNA
actgaaaggtcagattcaggag

SEQ ID NO: 10 - DNA, 20 bp, synthetic construct
  source 1..20: mol_type = other DNA
taccggtttgtgtccaagag

SEQ ID NO: 11 - DNA, 27 bp, synthetic construct
  source 1..27: mol_type = other DNA
tgaggctcgacaggtaccatatagcct

SEQ ID NO: 12 - DNA, 24 bp, synthetic construct
  source 1..24: mol_type = other DNA
gcctgtcgtgcagccatgagttag

SEQ ID NO: 13 - DNA, 34 bp, synthetic construct
  source 1..34: mol_type = other DNA
gactccaaccattggtataatctgaaagttgtct

SEQ ID NO: 14 - DNA, 29 bp, synthetic construct
  source 1..29: mol_type = other DNA
aaaggagaactatgtgagctaatgcattt

SEQ ID NO: 15 - DNA, 21 bp, synthetic construct
  source 1..21: mol_type = other DNA
ctgcctgacaacggactgaaa

SEQ ID NO: 16 - DNA, 29 bp, synthetic construct
  source 1..29: mol_type = other DNA
ctgtatatctggctagccatatgcagaaa

SEQ ID NO: 17 - DNA, 32 bp, synthetic construct
  source 1..32: mol_type = other DNA
tcggctatttctaacagctcacatgtttacat

SEQ ID NO: 18 - DNA, 30 bp, synthetic construct
  source 1..30: mol_type = other DNA
caatcagaagactgctagggtggctgtatt

SEQ ID NO: 19 - DNA, 29 bp, synthetic construct
  source 1..29: mol_type = other DNA
ttccataagtgccagctggctgagaaata

SEQ ID NO: 20 - DNA, 33 bp, synthetic construct
  source 1..33: mol_type = other DNA
gagtgtatagactagaagtacagccatatagtt

SEQ ID NO: 21 - DNA, 29 bp, synthetic construct
  source 1..29: mol_type = other DNA
tctgctcccaggttcaagtgattctcaag

SEQ ID NO: 22 - DNA, 22 bp, synthetic construct
  source 1..22: mol_type = other DNA
ccctgttgcccaggctagagtg

SEQ ID NO: 23 - DNA, 22 bp, synthetic construct
  source 1..22: mol_type = other DNA
tccgagagctcacagcttggct

SEQ ID NO: 24 - DNA, 27 bp, synthetic construct
  source 1..27: mol_type = other DNA
gagcgagctcaagatagaggcagctaa

SEQ ID NO: 25 - DNA, 26 bp, synthetic construct
  source 1..26: mol_type = other DNA
gtttgatggaattcaccagtgtgttt

SEQ ID NO: 26 - DNA, 21 bp, synthetic construct
  source 1..21: mol_type = other DNA
tccacatcacgggagcagctg

SEQ ID NO: 27 - DNA, 35 bp, synthetic construct
  source 1..35: mol_type = other DNA
atttagagagcctgtcacaaatgtccatatgagaa

SEQ ID NO: 28 - DNA, 29 bp, synthetic construct
  source 1..29: mol_type = other DNA
gtccagctgagtggaacattgaaagaaca

SEQ ID NO: 29 - DNA, 23 bp, synthetic construct
  source 1..23: mol_type = other DNA
gagaggagagagtgcagggtcaa

SEQ ID NO: 30 - DNA, 19 bp, synthetic construct
  source 1..19: mol_type = other DNA
taggaggagcccattggcc

SEQ ID NO: 31 - DNA, 34 bp, synthetic construct
  source 1..34: mol_type = other DNA
acattattgttaacaagggttgcttctgggaaga

SEQ ID NO: 32 - DNA, 23 bp, synthetic construct
  source 1..23: mol_type = other DNA
gcaggagaatggcatgaacccgg

SEQ ID NO: 33 - DNA, 24 bp, synthetic construct
  source 1..24: mol_type = other DNA
ccctggcacctagtgggtccatac

SEQ ID NO: 34 - DNA, 28 bp, synthetic construct
  source 1..28: mol_type = other DNA
agatgtggctgaactttaaatgcatatt

SEQ ID NO: 35 - DNA, 25 bp, synthetic construct
  source 1..25: mol_type = other DNA
gcactccagagactctgtttctaaa

SEQ ID NO: 36 - DNA, 21 bp, synthetic construct
  source 1..21: mol_type = other DNA
aaagggccaggcctgagtggt

SEQ ID NO: 37 - DNA, 19 bp, synthetic construct
  source 1..19: mol_type = other DNA
caggcatggtagcgcgcct

SEQ ID NO: 38 - DNA, 21 bp, synthetic construct
  source 1..21: mol_type = other DNA
ctccggcactgggcagaggta

SEQ ID NO: 39 - DNA, 28 bp, synthetic construct
  source 1..28: mol_type = other DNA
ggaagcctcataatcatggtggaaggca

SEQ ID NO: 40 - DNA, 27 bp, synthetic construct
  source 1..27: mol_type = other DNA
tcagtgctagctgataactctgacttt

SEQ ID NO: 41 - DNA, 24 bp, synthetic construct
  source 1..24: mol_type = other DNA
catgtttaccgatgaagctgcaaa

SEQ ID NO: 42 - DNA, 26 bp, synthetic construct
  source 1..26: mol_type = other DNA
cactgcatgttcccagtcataggtgg

SEQ ID NO: 43 - DNA, 33 bp, synthetic construct
  source 1..33: mol_type = other DNA
aggaagctcagctatacctgttactgataattt

SEQ ID NO: 44 - DNA, 24 bp, synthetic construct
  source 1..24: mol_type = other DNA
agacccatctcacatgtagacaca

SEQ ID NO: 45 - DNA, 23 bp, synthetic construct
  source 1..23: mol_type = other DNA
cccatgcaggcctaggctagtgt

SEQ ID NO: 46 - DNA, 29 bp, synthetic construct
  source 1..29: mol_type = other DNA
cttctccaagtctctacacttggccacaa

SEQ ID NO: 47 - DNA, 27 bp, synthetic construct
  source 1..27: mol_type = other DNA
acctaaaggacacatctcagtacattt

SEQ ID NO: 48 - DNA, 24 bp, synthetic construct
  source 1..24: mol_type = other DNA
aggatgacgcctggcttggagaat

SEQ ID NO: 49 - DNA, 27 bp, synthetic construct
  source 1..27: mol_type = other DNA
actgaaggctctatgtgcaggaactcc

SEQ ID NO: 50 - DNA, 35 bp, synthetic construct
  source 1..35: mol_type = other DNA
agcaacaaagattatcctaagacttctggaggaag

SEQ ID NO: 51 - DNA, 27 bp, synthetic construct
  source 1..27: mol_type = other DNA
cctcccaaagtgctgggattataggca

SEQ ID NO: 52 - DNA, 26 bp, synthetic construct
  source 1..26: mol_type = other DNA
aggagggagtgacaggatgtgatctg

SEQ ID NO: 53 - DNA, 23 bp, synthetic construct
  source 1..23: mol_type = other DNA
aactgttcttccaggcctgattt

SEQ ID NO: 54 - DNA, 23 bp, synthetic construct
  source 1..23: mol_type = other DNA
agcctggccaacacagtgaaacc

SEQ ID NO: 55 - RNA, 24 nt, synthetic construct
  source 1..24: mol_type = other RNA
cccatcctggcacgtactgctact

SEQ ID NO: 56 - RNA, 28 nt, synthetic construct
  source 1..28: mol_type = other RNA
tgacctgttcttgggtagcactattggt

SEQ ID NO: 57 - DNA, 31 bp, synthetic construct
  source 1..31: mol_type = other DNA
aggcacactcaggctttacgattcttgttta

SEQ ID NO: 58 - DNA, 30 bp, synthetic construct
  source 1..30: mol_type = other DNA
aaagggatagcagtttcctgctgtccatag

SEQ ID NO: 59 - DNA, 35 bp, synthetic construct
  source 1..35: mol_type = other DNA
attctccctgcaaattgtttgactctagaaatagc

SEQ ID NO: 60 - DNA, 22 bp, synthetic construct
  source 1..22: mol_type = other DNA
cccaggctgctggtcatggaaa

SEQ ID NO: 61 - DNA, 26 bp, synthetic construct
  source 1..26: mol_type = other DNA
gggtagtagcgatctgggttacccag

SEQ ID NO: 62 - DNA, 25 bp, synthetic construct
  source 1..25: mol_type = other DNA
cacagggtttatctcctcagacaaa

SEQ ID NO: 63 - DNA, 25 bp, synthetic construct
  source 1..25: mol_type = other DNA
cattgacccaccggtcattcaggag

SEQ ID NO: 64 - DNA, 17 bp, synthetic construct
  source 1..17: mol_type = other DNA
gcggacgccatggagct

SEQ ID NO: 65 - DNA, 28 bp, synthetic construct
  source 1..28: mol_type = other DNA
ctgagggttatccaagtgacccattgca

SEQ ID NO: 66 - DNA, 29 bp, synthetic construct
  source 1..29: mol_type = other DNA
aacacattagtgatgtctcaatgccatcc

SEQ ID NO: 67 - RNA, 26 nt, synthetic construct
  source 1..26: mol_type = other RNA
catggttgaacctgggaggcggaggt

SEQ ID NO: 68 - DNA, 22 bp, synthetic construct
  source 1..22: mol_type = other DNA
gctcaggcactgagagtcagcc

SEQ ID NO: 69 - RNA, 24 nt, synthetic construct
  source 1..24: mol_type = other RNA
catgcaggaggccaagggtgacgg

SEQ ID NO: 70 - DNA, 29 bp, synthetic construct
  source 1..29: mol_type = other DNA
ggatagctattgagagcttccttcatttg

SEQ ID NO: 71 - DNA, 39 bp, synthetic construct
  source 1..39: mol_type = other DNA
catgaagagtacttgacacatgcatgaactcagtaaatt

SEQ ID NO: 72 - DNA, 34 bp, synthetic construct
  source 1..34: mol_type = other DNA
atgaagtatcatataaggatacactctatgcatc

SEQ ID NO: 73 - DNA, 28 bp, synthetic construct
  source 1..28: mol_type = other DNA
catggggtcccacatgacagccatgact

SEQ ID NO: 74 - DNA, 24 bp, synthetic construct
  source 1..24: mol_type = other DNA
ggtcccagtccctcagtctctcca

SEQ ID NO: 75 - DNA, 39 bp, synthetic construct
  source 1..39: mol_type = other DNA
catgaaagaaacaaacttgtctcggtatccagaaagtgt

SEQ ID NO: 76 - DNA, 29 bp, synthetic construct
  source 1..29: mol_type = other DNA
acagttcagtagtgttaagaacatttgca

SEQ ID NO: 77 - DNA, 39 bp, synthetic construct
  source 1..39: mol_type = other DNA
catgaactattccttcaagccaagcatagtatgtctaga

SEQ ID NO: 78 - DNA, 24 bp, synthetic construct
  source 1..24: mol_type = other DNA
ggatggcacctgctggctacattt

SEQ ID NO: 79 - RNA, 31 nt, synthetic construct
  source 1..31: mol_type = other RNA
catgagtagttgggattacaggcatgtgcca

SEQ ID NO: 80 - RNA, 34 nt, synthetic construct
  source 1..34: mol_type = other RNA
aagacacaagtttatctatatgacaaactttgac

SEQ ID NO: 81 - DNA, 32 bp, synthetic construct
  source 1..32: mol_type = other DNA
catgtgtaactaacctgcacgttgtgcacatg

SEQ ID NO: 82 - DNA, 27 bp, synthetic construct
  source 1..27: mol_type = other DNA
tgcccattgtattagtcaggcagggtt

SEQ ID NO: 83 - DNA, 29 bp, synthetic construct
  source 1..29: mol_type = other DNA
catgctcctggactcaagcgatcctcctg

SEQ ID NO: 84 - DNA, 21 bp, synthetic construct
  source 1..21: mol_type = other DNA
ggagctggtggagcctgctct

SEQ ID NO: 85 - DNA, 38 bp, synthetic construct
  source 1..38: mol_type = other DNA
catgaatatgaaataaagcccacagtaaagattactct

SEQ ID NO: 86 - DNA, 34 bp, synthetic construct
  source 1..34: mol_type = other DNA
gtgaccaaactaaatgtttgtgtactttaaagga

SEQ ID NO: 87 - DNA, 26 bp, synthetic construct
  source 1..26: mol_type = other DNA
catggccactgcactccagcctaggt

SEQ ID NO: 88 - RNA, 24 nt, synthetic construct
  source 1..24: mol_type = other RNA
gtggccctaactcggctcaccata

SEQ ID NO: 89 - DNA, 36 bp, synthetic construct
  source 1..36: mol_type = other DNA
catgaaagaatatcgatacatgggcagctgacactt

SEQ ID NO: 90 - DNA, 28 bp, synthetic construct
  source 1..28: mol_type = other DNA
aaatagctgctttagatatctgaagcca

SEQ ID NO: 91 - DNA, 35 bp, synthetic construct
  source 1..35: mol_type = other DNA
catgtttgagacacagtttctctcttgtcacccag

SEQ ID NO: 92 - DNA, 26 bp, synthetic construct
  source 1..26: mol_type = other DNA
cccactgtggaatgtcacagctattt

SEQ ID NO: 93 - DNA, 26 bp, synthetic construct
  source 1..26: mol_type = other DNA
catgcccactgctggaccttagcacg

SEQ ID NO: 94 - DNA, 29 bp, synthetic construct
  source 1..29: mol_type = other DNA
tgatgcatggccttagctgtttactttgc

SEQ ID NO: 95 - DNA, 31 bp, synthetic construct
  source 1..31: mol_type = other DNA
catgctccacatgcatagtatcagccctcca

SEQ ID NO: 96 - DNA, 25 bp, synthetic construct
  source 1..25: mol_type = other DNA
gaggtggaaattgcagtgagccgag

SEQ ID NO: 97 - DNA, 24 bp, synthetic construct
  source 1..24: mol_type = other DNA
catgaggtgtgagccaccacgcct

SEQ ID NO: 98 - DNA, 26 bp, synthetic construct
  source 1..26: mol_type = other DNA
agtgactcacgcctataatcccagcg

SEQ ID NO: 99 - DNA, 32 bp, synthetic construct
  source 1..32: mol_type = other DNA
catgtttaaagacagtcttcgccaggtgtggt

SEQ ID NO: 100 - DNA, 19 bp, synthetic construct
  source 1..19: mol_type = other DNA
ccgaggcaggccgatcacc

SEQ ID NO: 101 - DNA, 35 bp, synthetic construct
  source 1..35: mol_type = other DNA
catgtttacgaggccagcatcatcctgatactaaa

SEQ ID NO: 102 - DNA, 34 bp, synthetic construct
  source 1..34: mol_type = other DNA
gtaacataatacaaagctgcatcacagctaatct

SEQ ID NO: 103 - DNA, 26 bp, synthetic construct
  source 1..26: mol_type = other DNA
catggggatcccagaccttggccagg

SEQ ID NO: 104 - DNA, 35 bp, synthetic construct
  source 1..35: mol_type = other DNA
aggtgcttataaatttctgttacatgaaagaatac

SEQ ID NO: 105 - DNA, 39 bp, synthetic construct
  source 1..39: mol_type = other DNA
catgaaattaagaaggttcatcacagttcaaagaagctc

SEQ ID NO: 106 - DNA, 32 bp, synthetic construct
  source 1..32: mol_type = other DNA
attcccgctcttatggcttatactctagttgg

SEQ ID NO: 107 - DNA, 27 bp, synthetic construct
  source 1..27: mol_type = other DNA
catgtggtctggtcgtccatgcttgca

SEQ ID NO: 108 - DNA, 28 bp, synthetic construct
  source 1..28: mol_type = other DNA
cacagttctcatactcaggaaccctcat

SEQ ID NO: 109 - DNA, 29 bp, synthetic construct
  source 1..29: mol_type = other DNA
catgtggtgttggcgctcagacagtgata

SEQ ID NO: 110 - DNA, 32 bp, synthetic construct
  source 1..32: mol_type = other DNA
tttgtcagttggccctgatttcagatatcctc

SEQ ID NO: 111 - DNA, 31 bp, synthetic construct
  source 1..31: mol_type = other DNA
catggcttgtaatcccagctactcaggaggc

SEQ ID NO: 112 - DNA, 27 bp, synthetic construct
  source 1..27: mol_type = other DNA
tttcaccagcacagtgtagaattgcag

SEQ ID NO: 113 - DNA, 26 bp, synthetic construct
  source 1..26: mol_type = other DNA
catgcttgtacctggtgttcggttat

SEQ ID NO: 114 - DNA, 24 bp, synthetic construct
  source 1..24: mol_type = other DNA
catgccagcccaagatggatgaat

SEQ ID NO: 115 - DNA, 25 bp, synthetic construct
  source 1..25: mol_type = other DNA
catggcggtggaagtcattctgata

SEQ ID NO: 116 - DNA, 24 bp, synthetic construct
  source 1..24: mol_type = other DNA
catggagggccacagattggtatt

SEQ ID NO: 117 - DNA, 26 bp, synthetic construct
  source 1..26: mol_type = other DNA
catgactgaaaggtcagattcaggag

SEQ ID NO: 118 - DNA, 39 bp, synthetic construct
  source 1..39: mol_type = other DNA
catgactacatttcctgctatcttgatttacaactcttg

SEQ ID NO: 119 - DNA, 28 bp, synthetic construct
  source 1..28: mol_type = other DNA
tccgaaggaagtgaattcacccaaggat

SEQ ID NO: 120 - DNA, 34 bp, synthetic construct
  source 1..34: mol_type = other DNA
catgtttgggatgaaactgttccacctcagattt

SEQ ID NO: 121 - DNA, 29 bp, synthetic construct
  source 1..29: mol_type = other DNA
ttactgccagagtattctggttaagaaac

SEQ ID NO: 122 - DNA, 39 bp, synthetic construct
  source 1..39: mol_type = other DNA
catgcatagtatttcacatttgtgtattacttcgaattt

SEQ ID NO: 123 - DNA, 34 bp, synthetic construct
  source 1..34: mol_type = other DNA
aaacgtttggttcattacaagaatagaggccttc

SEQ ID NO: 124 - DNA, 34 bp, synthetic construct
  source 1..34: mol_type = other DNA
catgtccttgagtctatcttggaggtgagcaaac

SEQ ID NO: 125 - DNA, 29 bp, synthetic construct
  source 1..29: mol_type = other DNA
tgcgtggtgtgcagtaaattatcaatagg

SEQ ID NO: 126 - DNA, 39 bp, synthetic construct
  source 1..39: mol_type = other DNA
catgccaaatttaattcagtctttcttcccatagtgtct

SEQ ID NO: 127 - DNA, 35 bp, synthetic construct
  source 1..35: mol_type = other DNA
actctttatgaagagcaatttggtagtacctacca

SEQ ID NO: 128 - DNA, 31 bp, synthetic construct
  source 1..31: mol_type = other DNA
catggttcaacaccctttgatggctcccatg

SEQ ID NO: 129 - DNA, 24 bp, synthetic construct
  source 1..24: mol_type = other DNA
ggcctccttgaactatggtgggct

SEQ ID NO: 130 - DNA, 33 bp, synthetic construct
  source 1..33: mol_type = other DNA
catgaaagcagtgtgctgagtgaaagaagtcag

SEQ ID NO: 131 - DNA, 29 bp, synthetic construct
  source 1..29: mol_type = other DNA
tcctcatgtgagtttatgtcctacttgaa

SEQ ID NO: 132 - DNA, 31 bp, synthetic construct
  source 1..31: mol_type = other DNA
catgttgtgctgcaataaacatagatgcgcg

SEQ ID NO: 133 - DNA, 29 bp, synthetic construct
  source 1..29: mol_type = other DNA
agtccaaagtttaagatgccagagatgca

SEQ ID NO: 134 - DNA, 39 bp, synthetic construct
  source 1..39: mol_type = other DNA
catgtttctccatagactcctgttcttgttatacagatg

SEQ ID NO: 135 - DNA, 35 bp, synthetic construct
  source 1..35: mol_type = other DNA
ttgatataaagcctactgtgatcttggcaccatac

SEQ ID NO: 136 - DNA, 39 bp, synthetic construct
  source 1..39: mol_type = other DNA
catgttgtggatgattagcatcttaaccatgttgagtct

SEQ ID NO: 137 - DNA, 25 bp, synthetic construct
  source 1..25: mol_type = other DNA
tcaggagtttgagatcagcctggcc

SEQ ID NO: 138 - DNA, 31 bp, synthetic construct
  source 1..31: mol_type = other DNA
catgcccagatgtggatgttcatggtccaga

SEQ ID NO: 139 - DNA, 25 bp, synthetic construct
  source 1..25: mol_type = other DNA
gggtggatgagactttccgaagaac

SEQ ID NO: 140 - DNA, 23 bp, synthetic construct
  source 1..23: mol_type = other DNA
catgccctgcgaaccgtcgaccg

SEQ ID NO: 141 - DNA, 29 bp, synthetic construct
  source 1..29: mol_type = other DNA
gtggattacctgaggtcagtagttcaaga

SEQ ID NO: 142 - DNA, 23 bp, synthetic construct
  source 1..23: mol_type = other DNA
catggcaggctgctggaccctgc

SEQ ID NO: 143 - DNA, 27 bp, synthetic construct
  source 1..27: mol_type = other DNA
cctggctagctccgaaatattccagga

SEQ ID NO: 144 - DNA, 28 bp, synthetic construct
  source 1..28: mol_type = other DNA
catgggttccttcgctactctggctcca

SEQ ID NO: 145 - DNA, 21 bp, synthetic construct
  source 1..21: mol_type = other DNA
ccaccacacccagctaagttt

SEQ ID NO: 146 - DNA, 39 bp, synthetic construct
  source 1..39: mol_type = other DNA
catggaatctcaaacggtatttaacatataagaagattc

SEQ ID NO: 147 - DNA, 35 bp, synthetic construct
  source 1..35: mol_type = other DNA
ctgcatagaatacagccttactgaatgttagtctc

SEQ ID NO: 148 - DNA, 27 bp, synthetic construct
  source 1..27: mol_type = other DNA
catgaaggctcctggagccctttgagg

SEQ ID NO: 149 - DNA, 27 bp, synthetic construct
  source 1..27: mol_type = other DNA
tctgtctgcctggagcactgatttctc

SEQ ID NO: 150 - DNA, 35 bp, synthetic construct
  source 1..35: mol_type = other DNA
catgtattggatagtgagcccaccactcagtaaca

SEQ ID NO: 151 - DNA, 29 bp, synthetic construct
  source 1..29: mol_type = other DNA
cctgagctagatataaagactctccacgt

SEQ ID NO: 152 - DNA, 25 bp, synthetic construct
  source 1..25: mol_type = other DNA
catgaccaccacgcctggctaattt

SEQ ID NO: 153 - DNA, 27 bp, synthetic construct
  source 1..27: mol_type = other DNA
tttaagccaatagcaccatgtttcttt

SEQ ID NO: 154 - DNA, 24 bp, synthetic construct
  source 1..24: mol_type = other DNA
catggggcattgcagcaggtgcag

SEQ ID NO: 155 - DNA, 24 bp, synthetic construct
  source 1..24: mol_type = other DNA
ggcatttattgcagggtgccaagc

SEQ ID NO: 156 - DNA, 31 bp, synthetic construct
  source 1..31: mol_type = other DNA
catgccttaaaccatcacggtgctgctttga

SEQ ID NO: 157 - DNA, 26 bp, synthetic construct
  source 1..26: mol_type = other DNA
agcctatgtgtgtctctgcacgtgat

SEQ ID NO: 158 - DNA, 38 bp, synthetic construct
  source 1..38: mol_type = other DNA
catgatcttacaagaagacatatgcccacagaggacta

SEQ ID NO: 159 - DNA, 29 bp, synthetic construct
  source 1..29: mol_type = other DNA
ctgctgagaaattactgtccctgtgcaaa

SEQ ID NO: 160 - DNA, 30 bp, synthetic construct
  source 1..30: mol_type = other DNA
catgctgggcatcagagcaagactccatct

SEQ ID NO: 161 - DNA, 18 bp, synthetic construct
  source 1..18: mol_type = other DNA
cccgtgccacactcgccg

SEQ ID NO: 162 - DNA, 27 bp, synthetic construct
  source 1..27: mol_type = other DNA
catgctgggcatgcgacatctctcagg

SEQ ID NO: 163 - DNA, 34 bp, synthetic construct
  source 1..34: mol_type = other DNA
atcgtcaaatgaactcgaaatgaatcataatcga

SEQ ID NO: 164 - DNA, 25 bp, synthetic construct
  source 1..25: mol_type = other DNA
catgttgccctggtgcctaggctgg

SEQ ID NO: 165 - DNA, 28 bp, synthetic construct
  source 1..28: mol_type = other DNA
gcacaaccagagatacatcagccctaaa

SEQ ID NO: 166 - DNA, 25 bp, synthetic construct
  source 1..25: mol_type = other DNA
catggcccaccttggcctcccaaag

SEQ ID NO: 167 - DNA, 35 bp, synthetic construct
  source 1..35: mol_type = other DNA
gaatgcttgtgtttaaatcttcagtgtcactctag

SEQ ID NO: 168 - DNA, 30 bp, synthetic construct
  source 1..30: mol_type = other DNA
catggggaatagacatgctgtggtggcaga

SEQ ID NO: 169 - DNA, 28 bp, synthetic construct
  source 1..28: mol_type = other DNA
agctaatatcatgccactgcactccagc

SEQ ID NO: 170 - DNA, 35 bp, synthetic construct
  source 1..35: mol_type = other DNA
catgattaaggatgaggaccctgaggttcagaaac

SEQ ID NO: 171 - DNA, 25 bp, synthetic construct
  source 1..25: mol_type = other DNA
aggcttcaccagacaccaaatctgc

SEQ ID NO: 172 - DNA, 32 bp, synthetic construct
  source 1..32: mol_type = other DNA
catgagatctgctgttagtctgatgggcttcc

SEQ ID NO: 173 - DNA, 19 bp, synthetic construct
  source 1..19: mol_type = other DNA
cctatgcggccgagcctcc

SEQ ID NO: 174 - DNA, 32 bp, synthetic construct
  source 1..32: mol_type = other DNA
catgtttcagccaagcagtttgaaaggattct

SEQ ID NO: 175 - DNA, 28 bp, synthetic construct
  source 1..28: mol_type = other DNA
ggtctcaaactcctgggttcaagtgaac

SEQ ID NO: 176 - DNA, 34 bp, synthetic construct
  source 1..34: mol_type = other DNA
catgattatgataggtatcaacacccttgcattt

SEQ ID NO: 177 - DNA, 25 bp, synthetic construct
  source 1..25: mol_type = other DNA
acgaatttgtggccaagtcctcaaa

SEQ ID NO: 178 - DNA, 34 bp, synthetic construct
  source 1..34: mol_type = other DNA
catgggaatctttgttaattgtttcacgtggaaa

SEQ ID NO: 179 - DNA, 27 bp, synthetic construct
  source 1..27: mol_type = other DNA
ggctgtggagatgcatcagtgaatcag

SEQ ID NO: 180 - DNA, 38 bp, synthetic construct
  source 1..38: mol_type = other DNA
catgtccattgccattactatttcagagttacaatgtt

SEQ ID NO: 181 - DNA, 29 bp, synthetic construct
  source 1..29: mol_type = other DNA
ttctcatgacagtgctgatctgtggattt

SEQ ID NO: 182 - DNA, 24 bp, synthetic construct
  source 1..24: mol_type = other DNA
catgcgagcggcccaactccacag

SEQ ID NO: 183 - DNA, 25 bp, synthetic construct
  source 1..25: mol_type = other DNA
tctcagctactcgggaggctgagat

SEQ ID NO: 184 - DNA, 35 bp, synthetic construct
  source 1..35: mol_type = other DNA
catgaaacgagttaatacttgcaccgtctcagaca

SEQ ID NO: 185 - DNA, 26 bp, synthetic construct
  source 1..26: mol_type = other DNA
tcatggcatggatgtaccaaagattt

SEQ ID NO: 186 - DNA, 38 bp, synthetic construct
  source 1..38: mol_type = other DNA
catgaaacacaaccactgagagtgctattatggtactc

SEQ ID NO: 187 - DNA, 29 bp, synthetic construct
  source 1..29: mol_type = other DNA
tcctgagccaggtttacatggttaccatt

SEQ ID NO: 188 - DNA, 33 bp, synthetic construct
  source 1..33: mol_type = other DNA
catgggacattcatgtttctccaaagccctctg

SEQ ID NO: 189 - DNA, 22 bp, synthetic construct
  source 1..22: mol_type = other DNA
gtcaccatggccactctggagc

SEQ ID NO: 190 - DNA, 38 bp, synthetic construct
  source 1..38: mol_type = other DNA
catggaagttgccaaacagttcctcagaatagttacac

SEQ ID NO: 191 - DNA, 26 bp, synthetic construct
  source 1..26: mol_type = other DNA
atctatcccaaagggctgctgtgagg

SEQ ID NO: 192 - DNA, 37 bp, synthetic construct
  source 1..37: mol_type = other DNA
catgttgaatgaatggtacgtgcgaagtatatgctga

SEQ ID NO: 193 - DNA, 23 bp, synthetic construct
  source 1..23: mol_type = other DNA
gtctgggaggcagaggttgcagt

SEQ ID NO: 194 - DNA, 39 bp, synthetic construct
  source 1..39: mol_type = other DNA
catgccacaaacctctttgttataatttctccagttgag

SEQ ID NO: 195 - DNA, 20 bp, synthetic construct
  source 1..20: mol_type = other DNA
ctgcctgctggcagggcttc

SEQ ID NO: 196 - DNA, 39 bp, synthetic construct
  source 1..39: mol_type = other DNA
catggggatagacagacctgttgaatcataaatagaaca

SEQ ID NO: 197 - DNA, 32 bp, synthetic construct
  source 1..32: mol_type = other DNA
cagattcgctaaatgattgaccttgtacattt

SEQ ID NO: 198 - DNA, 30 bp, synthetic construct
  source 1..30: mol_type = other DNA
catgaccttcacaaagcatgcactgactgt

SEQ ID NO: 199 - DNA, 26 bp, synthetic construct
  source 1..26: mol_type = other DNA
tgagctgtggccacttactcaggaaa

SEQ ID NO: 200 - DNA, 25 bp, synthetic construct
  source 1..25: mol_type = other DNA
catgatcccagcactctggagggcc

SEQ ID NO: 201 - DNA, 22 bp, synthetic construct
  source 1..22: mol_type = other DNA
tggtccctctggccaacttggc

SEQ ID NO: 202 - RNA, 36 nt, synthetic construct
  source 1..36: mol_type = other RNA
catgtttgcctaccttcctgtacacataaaccaagg

SEQ ID NO: 203 - DNA, 25 bp, synthetic construct
  source 1..25: mol_type = other DNA
ccaacagatgcctcccactccaagt

SEQ ID NO: 204 - DNA, 39 bp, synthetic construct
  source 1..39: mol_type = other DNA
catgttacacattattcagcaatggactagaattggtgc

SEQ ID NO: 205 - DNA, 22 bp, synthetic construct
  source 1..22: mol_type = other DNA
cacccaccttggcctcccaaag

SEQ ID NO: 206 - DNA, 32 bp, synthetic construct
  source 1..32: mol_type = other DNA
catgcatggcagcatggcagctttatatcctg

SEQ ID NO: 207 - DNA, 21 bp, synthetic construct
  source 1..21: mol_type = other DNA
ctcctgcctcagcctcccgag

SEQ ID NO: 208 - DNA, 25 bp, synthetic construct
  source 1..25: mol_type = other DNA
catggggcttggccttaagtgccgt

SEQ ID NO: 209 - DNA, 27 bp, synthetic construct
  source 1..27: mol_type = other DNA
gacagagtttcactcttgttgcccagg

SEQ ID NO: 210 - RNA, 25 nt, synthetic construct
  source 1..25: mol_type = other RNA
catggggtccaggaaagtcgggcag

SEQ ID NO: 211 - DNA, 25 bp, synthetic construct
  source 1..25: mol_type = other DNA
gagttcaagaccagcctcgccaatg

SEQ ID NO: 212 - DNA, 31 bp, synthetic construct
  source 1..31: mol_type = other DNA
catgttcacagctaatctctcctgggcacac

SEQ ID NO: 213 - DNA, 29 bp, synthetic construct
  source 1..29: mol_type = other DNA
gatggcttggaaatccataagtcctattt

SEQ ID NO: 214 - DNA, 39 bp, synthetic construct
  source 1..39: mol_type = other DNA
catgtttaacctgctcagtctagttagaacatctctcgt

SEQ ID NO: 215 - DNA, 24 bp, synthetic construct
  source 1..24: mol_type = other DNA
tgatcccacctggtctgatgggtg

SEQ ID NO: 216 - DNA, 34 bp, synthetic construct
  source 1..34: mol_type = other DNA
catgcacagagcatgagcattgaagcagaatgaa

SEQ ID NO: 217 - DNA, 34 bp, synthetic construct
  source 1..34: mol_type = other DNA
ggtgctcagtattctaattctttgattccttgtc

SEQ ID NO: 218 - DNA, 28 bp, synthetic construct
  source 1..28: mol_type = other DNA
catgcacacacaccagcatgcacactca

SEQ ID NO: 219 - RNA, 26 nt, synthetic construct
  source 1..26: mol_type = other RNA
ccagcatgatgttggtccaagttggc

SEQ ID NO: 220 - DNA, 24 bp, synthetic construct
  source 1..24: mol_type = other DNA
catggggccaaagcggactgaggc

SEQ ID NO: 221 - DNA, 31 bp, synthetic construct
  source 1..31: mol_type = other DNA
gccgttgttcttatcatctgtcgataccaaa

SEQ ID NO: 222 - RNA, 35 nt, synthetic construct
  source 1..35: mol_type = other RNA
catgaaagacccagaatagccaaagctatcctgag

SEQ ID NO: 223 - DNA, 24 bp, synthetic construct
  source 1..24: mol_type = other DNA
tccgtgtgcccaagtcagttacca

SEQ ID NO: 224 - DNA, 32 bp, synthetic construct
  source 1..32: mol_type = other DNA
catgtggtcctttcttgcagtaggaggatgtc

SEQ ID NO: 225 - DNA, 28 bp, synthetic construct
  source 1..28: mol_type = other DNA
taattggtcagtatccgtatgggtcttt

SEQ ID NO: 226 - DNA, 31 bp, synthetic construct
  source 1..31: mol_type = other DNA
catgaaactgcctgattccacagctgaggta

SEQ ID NO: 227 - DNA, 29 bp, synthetic construct
  source 1..29: mol_type = other DNA
gagccttctaccttcagactcagactgaa

SEQ ID NO: 228 - RNA, 30 nt, synthetic construct
  source 1..30: mol_type = other RNA
catggggatgcttggtgcaggcttctattt

SEQ ID NO: 229 - DNA, 28 bp, synthetic construct
  source 1..28: mol_type = other DNA
tgttggctcctatgtccatttgacattt

SEQ ID NO: 230 - DNA, 33 bp, synthetic construct
  source 1..33: mol_type = other DNA
catgggaaatccctcagtccggaagtctagaac

SEQ ID NO: 231 - DNA, 35 bp, synthetic construct
  source 1..35: mol_type = other DNA
tgcctgtgtatatacagcatgtatgatgtacattt

SEQ ID NO: 232 - DNA, 39 bp, synthetic construct
  source 1..39: mol_type = other DNA
catggcacatgcaaataatgcaatcatatatggcttaat

SEQ ID NO: 233 - DNA, 34 bp, synthetic construct
  source 1..34: mol_type = other DNA
gtatcgaagtatataaactgatggttcacagaga

SEQ ID NO: 234 - DNA, 39 bp, synthetic construct
  source 1..39: mol_type = other DNA
catgtaccataacttaatgccatatttgccatagttggc

SEQ ID NO: 235 - DNA, 26 bp, synthetic construct
  source 1..26: mol_type = other DNA
cacgcaaccgtaggtgtgtattccag

SEQ ID NO: 236 - DNA, 36 bp, synthetic construct
  source 1..36: mol_type = other DNA
catgaaatgtaaaccttgtctaaatctgggatcttc

SEQ ID NO: 237 - DNA, 34 bp, synthetic construct
  source 1..34: mol_type = other DNA
tttgtctctgcttacatttggctctctgtaattt

SEQ ID NO: 238 - DNA, 39 bp, synthetic construct
  source 1..39: mol_type = other DNA
catgaaagctcacaattacaagtgctaagtcttcagaaa

SEQ ID NO: 239 - DNA, 32 bp, synthetic construct
  source 1..32: mol_type = other DNA
cctgtatttctgtgtcttcacatggcattctc

SEQ ID NO: 240 - DNA, 37 bp, synthetic construct
  source 1..37: mol_type = other DNA
catgctaaatcctttcccgttagggactgttctgatc

SEQ ID NO: 241 - DNA, 34 bp, synthetic construct
  source 1..34: mol_type = other DNA
gctgcataaatgtcttctttagagaagtgtcttt

SEQ ID NO: 242 - DNA, 31 bp, synthetic construct
  source 1..31: mol_type = other DNA
catgaatggtggaggtagcagcattgcaatc

SEQ ID NO: 243 - DNA, 25 bp, synthetic construct
  source 1..25: mol_type = other DNA
cttgaactcctgggctcaagcgatc

SEQ ID NO: 244 - DNA, 39 bp, synthetic construct
  source 1..39: mol_type = other DNA
catgttactcattcttaagcactaggtgtgattctaaat

SEQ ID NO: 245 - DNA, 25 bp, synthetic construct
  source 1..25: mol_type = other DNA
catcatcccaccaagttagctccaa

SEQ ID NO: 246 - DNA, 35 bp, synthetic construct
  source 1..35: mol_type = other DNA
catgttaaggcatagcttagcagtccaccaaagat

SEQ ID NO: 247 - DNA, 26 bp, synthetic construct
  source 1..26: mol_type = other DNA
tcccaggtaatgctgatgttgccagt

SEQ ID NO: 248 - RNA, 39 nt, synthetic construct
  source 1..39: mol_type = other RNA
catgaatgcttaagatatgtacccagcataccttaaatg

SEQ ID NO: 249 - DNA, 35 bp, synthetic construct
  source 1..35: mol_type = other DNA
caagctcaagatttctgttagttaccagtttcttt

SEQ ID NO: 250 - DNA, 35 bp, synthetic construct
  source 1..35: mol_type = other DNA
catgaaagtcacactgttgctaacaagctgataaa

SEQ ID NO: 251 - RNA, 25 nt, synthetic construct
  source 1..25: mol_type = other RNA
gggctctggtgtgacgaagtcattg

SEQ ID NO: 252 - DNA, 23 bp, synthetic construct
  source 1..23: mol_type = other DNA
catgctgcactccagcctgggcg

SEQ ID NO: 253 - DNA, 29 bp, synthetic construct
  source 1..29: mol_type = other DNA
taggagtgcttctctgatgatgttccacc

SEQ ID NO: 254 - DNA, 39 bp, synthetic construct
  source 1..39: mol_type = other DNA
catgcatattatatctttgcttgcctagtttctcatgtt

SEQ ID NO: 255 - DNA, 28 bp, synthetic construct
  source 1..28: mol_type = other DNA
ttatggttattacttgacagagggcagt

SEQ ID NO: 256 - RNA, 32 nt, synthetic construct
  source 1..32: mol_type = other RNA
catgatggtagcatgacttcctggcctctagt

SEQ ID NO: 257 - DNA, 29 bp, synthetic construct
  source 1..29: mol_type = other DNA
ttggtgctttatggtttcattctgaacac

SEQ ID NO: 258 - DNA, 30 bp, synthetic construct
  source 1..30: mol_type = other DNA
catgttggcaatggcatttggtttcctcca

SEQ ID NO: 259 - DNA, 29 bp, synthetic construct
  source 1..29: mol_type = other DNA
atgagatagatgacagaactgacacctag

SEQ ID NO: 260 - DNA, 31 bp, synthetic construct
  source 1..31: mol_type = other DNA
catgtagtctcagctacttgggaagctgagg

SEQ ID NO: 261 - DNA, 24 bp, synthetic construct
  source 1..24: mol_type = other DNA
tcctggcatttgggaagcttgcac

SEQ ID NO: 262 - DNA, 36 bp, synthetic construct
  source 1..36: mol_type = other DNA
catgtaagtggagataagcttgtatgtcttatatgg

SEQ ID NO: 263 - DNA, 28 bp, synthetic construct
  source 1..28: mol_type = other DNA
ggaggagggcatttgaggagtctctatg

SEQ ID NO: 264 - DNA, 34 bp, synthetic construct
  source 1..34: mol_type = other DNA
catgtttattggagggccaggtaatagctttgca

SEQ ID NO: 265 - DNA, 26 bp, synthetic construct
  source 1..26: mol_type = other DNA
agtgtgatagtgtgcgcctgtcaact

SEQ ID NO: 266 - DNA, 32 bp, synthetic construct
  source 1..32: mol_type = other DNA
catgtgaaagtccacctgtgagttgcagaact

SEQ ID NO: 267 - DNA, 24 bp, synthetic construct
  source 1..24: mol_type = other DNA
acctagcctttgctctagcaccgg

SEQ ID NO: 268 - DNA, 25 bp, synthetic construct
  source 1..25: mol_type = other DNA
catggggtgtggtggctcatggctg

SEQ ID NO: 269 - DNA, 32 bp, synthetic construct
  source 1..32: mol_type = other DNA
catgcagaagagtgtgttgccttccactgttc

SEQ ID NO: 270 - DNA, 39 bp, synthetic construct
  source 1..39: mol_type = other DNA
catgcaaacttgcttagttgtttacctgtgatattggct

SEQ ID NO: 271 - DNA, 34 bp, synthetic construct
  source 1..34: mol_type = other DNA
catgaccatttaatccaagctacccgctatgtcg

SEQ ID NO: 272 - DNA, 35 bp, synthetic construct
  source 1..35: mol_type = other DNA
catggctgaactggcctaacctgaatgtatgtaca

SEQ ID NO: 273 - DNA, 38 bp, synthetic construct
  source 1..38: mol_type = other DNA
catggtatttgtatggtcatacacgaggaactgctgta

SEQ ID NO: 274 - DNA, 24 bp, synthetic construct
  source 1..24: mol_type = other DNA
ggttcaactgaagtgccagcctgc

SEQ ID NO: 275 - RNA, 32 nt, synthetic construct
  source 1..32: mol_type = other RNA
catgaaactcctgacctcaagtgatccacctg

SEQ ID NO: 276 - DNA, 27 bp, synthetic construct
  source 1..27: mol_type = other DNA
cctcaaagtgtctgcagagcaggaatc

SEQ ID NO: 277 - DNA, 39 bp, synthetic construct
  source 1..39: mol_type = other DNA
catgttatggctcaggataaattcctacatgtagaattc

SEQ ID NO: 278 - DNA, 29 bp, synthetic construct
  source 1..29: mol_type = other DNA
gaaggatcagagtaccagcatccgtcttc

SEQ ID NO: 279 - DNA, 29 bp, synthetic construct
  source 1..29: mol_type = other DNA
catggggtttcaccatgttggccaggatg

SEQ ID NO: 280 - DNA, 29 bp, synthetic construct
  source 1..29: mol_type = other DNA
cagtttatattccagataggcaggaggtt

SEQ ID NO: 281 - DNA, 39 bp, synthetic construct
  source 1..39: mol_type = other DNA
catgaataccagtaagagcttgaactttgtaagttaaac

SEQ ID NO: 282 - DNA, 22 bp, synthetic construct
  source 1..22: mol_type = other DNA
ctgtgcccatttcacgccctgg

SEQ ID NO: 283 - DNA, 27 bp, synthetic construct
  source 1..27: mol_type = other DNA
catgtgtgcagcagacttctgcctgga

SEQ ID NO: 284 - DNA, 28 bp, synthetic construct
  source 1..28: mol_type = other DNA
tttgtgaaaggcaaatgatgcatgacag

SEQ ID NO: 285 - DNA, 32 bp, synthetic construct
  source 1..32: mol_type = other DNA
catgtacagtattaggcacctgactgggtaaa

SEQ ID NO: 286 - DNA, 29 bp, synthetic construct
  source 1..29: mol_type = other DNA
gaaaggttaagcaacttgcccatggtcaa

SEQ ID NO: 287 - DNA, 28 bp, synthetic construct
  source 1..28: mol_type = other DNA
catggtggcacatgcctgtaatcccagc

SEQ ID NO: 288 - DNA, 21 bp, synthetic construct
  source 1..21: mol_type = other DNA
aggcccagccatctcactgca

SEQ ID NO: 289 - DNA, 34 bp, synthetic construct
  source 1..34: mol_type = other DNA
catggataatctgggtaccagccttaaggaaggc

SEQ ID NO: 290 - DNA, 29 bp, synthetic construct
  source 1..29: mol_type = other DNA
gggcccataggatttctggactgaaatca

SEQ ID NO: 291 - DNA, 32 bp, synthetic construct
  source 1..32: mol_type = other DNA
catgttgagctaagccagtccatcaagactgg

SEQ ID NO: 292 - DNA, 23 bp, synthetic construct
  source 1..23: mol_type = other DNA
gagcgattctcctgcctaggcct

SEQ ID NO: 293 - DNA, 25 bp, synthetic construct
  source 1..25: mol_type = other DNA
catgtgcctcggcctcccaagtagc

SEQ ID NO: 294 - DNA, 29 bp, synthetic construct
  source 1..29: mol_type = other DNA
acagtaaccagatctcagccaatcagagg

SEQ ID NO: 295 - DNA, 30 bp, synthetic construct
  source 1..30: mol_type = other DNA
catgaaattagctggtggcaggcacctgta

SEQ ID NO: 296 - DNA, 26 bp, synthetic construct
  source 1..26: mol_type = other DNA
ggttacagtgagccatgattgtgccg

SEQ ID NO: 297 - DNA, 39 bp, synthetic construct
  source 1..39: mol_type = other DNA
catgatttcgagaaataacttccgatttatttcagatag

SEQ ID NO: 298 - DNA, 28 bp, synthetic construct
  source 1..28: mol_type = other DNA
tagggcatttcacatgggctcagaaaca

SEQ ID NO: 299 - DNA, 27 bp, synthetic construct
  source 1..27: mol_type = other DNA
catgaagccaaggctgagccaagtcag

SEQ ID NO: 300 - DNA, 29 bp, synthetic construct
  source 1..29: mol_type = other DNA
aaacttccagactgattccaaagtccaag

SEQ ID NO: 301 - DNA, 29 bp, synthetic construct
  source 1..29: mol_type = other DNA
catgtcattgattccagtgcggtcctaaa

SEQ ID NO: 302 - DNA, 27 bp, synthetic construct
  source 1..27: mol_type = other DNA
ggtgacagagggagactctgtcacaaa

SEQ ID NO: 303 - DNA, 23 bp, synthetic construct
  source 1..23: mol_type = other DNA
catgcgcccggacagtggagacg

SEQ ID NO: 304 - DNA, 28 bp, synthetic construct
  source 1..28: mol_type = other DNA
tcttcctgtgttacctaggctggtctca

SEQ ID NO: 305 - DNA, 33 bp, synthetic construct
  source 1..33: mol_type = other DNA
catgtgtcccactggagaaccctgactaataca

SEQ ID NO: 306 - DNA, 27 bp, synthetic construct
  source 1..27: mol_type = other DNA
gctaggattacaggcgtgagccactat

SEQ ID NO: 307 - DNA, 38 bp, synthetic construct
  source 1..38: mol_type = other DNA
catgcttctacttgttggacttctgatagggtccaaag

SEQ ID NO: 308 - DNA, 29 bp, synthetic construct
  source 1..29: mol_type = other DNA
agtcatggaggtagagccttcttgaaagg

SEQ ID NO: 309 - DNA, 29 bp, synthetic construct
  source 1..29: mol_type = other DNA
catgcctcacaggtaggcgtgaggatcag

SEQ ID NO: 310 - DNA, 22 bp, synthetic construct
  source 1..22: mol_type = other DNA
gccctgaggctcacgtcactca

SEQ ID NO: 311 - DNA, 34 bp, synthetic construct
  source 1..34: mol_type = other DNA
catgctcaacatgaagacaacagggatgaggagc

SEQ ID NO: 312 - DNA, 35 bp, synthetic construct
  source 1..35: mol_type = other DNA
atgaagctaggattattaacctaagggtcacttgt

SEQ ID NO: 313 - DNA, 25 bp, synthetic construct
  source 1..25: mol_type = other DNA
catggtcgctcaggctggtgtgcag

SEQ ID NO: 314 - DNA, 28 bp, synthetic construct
  source 1..28: mol_type = other DNA
aaacacagtccctgtccttacagagctc

SEQ ID NO: 315 - DNA, 35 bp, synthetic construct
  source 1..35: mol_type = other DNA
catgggcttacatctgtaatcccagcactttgaga

SEQ ID NO: 316 - DNA, 27 bp, synthetic construct
  source 1..27: mol_type = other DNA
actgcctcagttaaccagcagacttca

SEQ ID NO: 317 - DNA, 25 bp, synthetic construct
  source 1..25: mol_type = other DNA
catgcctcccaccttggcctcccaa

SEQ ID NO: 318 - DNA, 24 bp, synthetic construct
  source 1..24: mol_type = other DNA
tcctgggctcaagcaatcctcctg

SEQ ID NO: 319 - DNA, 33 bp, synthetic construct
  source 1..33: mol_type = other DNA
catgatggatcctggtacccagagggaatacaa

SEQ ID NO: 320 - DNA, 26 bp, synthetic construct
  source 1..26: mol_type = other DNA
ggagcttgtacagcagctaaggtgag

SEQ ID NO: 321 - DNA, 32 bp, synthetic construct
  source 1..32: mol_type = other DNA
catggactcaaactcctgggctcaagtgatcc

SEQ ID NO: 322 - DNA, 29 bp, synthetic construct
  source 1..29: mol_type = other DNA
tccctgagatcaagtgattgtcccatctt

SEQ ID NO: 323 - DNA, 32 bp, synthetic construct
  source 1..32: mol_type = other DNA
catgcccatggactcgtaattggtttcctgga

SEQ ID NO: 324 - DNA, 29 bp, synthetic construct
  source 1..29: mol_type = other DNA
aatactctggatgatcttcaggacatgtt

SEQ ID NO: 325 - DNA, 31 bp, synthetic construct
  source 1..31: mol_type = other DNA
catggtgatagcatgcctctgtagtcccagc

SEQ ID NO: 326 - DNA, 21 bp, synthetic construct
  source 1..21: mol_type = other DNA
ggaggtggaggcgggaggatt

SEQ ID NO: 327 - RNA, 29 nt, synthetic construct
  source 1..29: mol_type = other RNA
catgaatgagtgagtgacgaggctgctgg

SEQ ID NO: 328 - DNA, 27 bp, synthetic construct
  source 1..27: mol_type = other DNA
aacccttcatgaacgtgaggaagctga

SEQ ID NO: 329 - DNA, 37 bp, synthetic construct
  source 1..37: mol_type = other DNA
catgtcttagccattcagatcaatcccagacagacta

SEQ ID NO: 330 - DNA, 29 bp, synthetic construct
  source 1..29: mol_type = other DNA
aatactgtagccttcctagggctctcaca

SEQ ID NO: 331 - RNA, 38 nt, synthetic construct
  source 1..38: mol_type = other RNA
catgtacaccttaacctagcaatgctttctcttctgag

SEQ ID NO: 332 - DNA, 23 bp, synthetic construct
  source 1..23: mol_type = other DNA
ctgccacagcctcccatgtagct

SEQ ID NO: 333 - DNA, 27 bp, synthetic construct
  source 1..27: mol_type = other DNA
catgtctgcctcagcctcccatgtagc

SEQ ID NO: 334 - DNA, 30 bp, synthetic construct
  source 1..30: mol_type = other DNA
ctaaggagagactgctttctgatgcctacc

SEQ ID NO: 335 - DNA, 27 bp, synthetic construct
  source 1..27: mol_type = other DNA
catgcatgcattgcaacgcagtggtgc

SEQ ID NO: 336 - DNA, 30 bp, synthetic construct
  source 1..30: mol_type = other DNA
cagtgctatccagcccagatactatgcttt

SEQ ID NO: 337 - DNA, 34 bp, synthetic construct
  source 1..34: mol_type = other DNA
catgctgcgaacacttaatgcagagcttgatacc

SEQ ID NO: 338 - DNA, 29 bp, synthetic construct
  source 1..29: mol_type = other DNA
aggatggtatcgatctcttcacctcgtga

SEQ ID NO: 339 - DNA, 36 bp, synthetic construct
  source 1..36: mol_type = other DNA
catgcttccagctagtttctagaggtcttgtctgga

SEQ ID NO: 340 - DNA, 33 bp, synthetic construct
  source 1..33: mol_type = other DNA
aagttgcaaactgcattgagatagacaattaaa

SEQ ID NO: 341 - DNA, 29 bp, synthetic construct
  source 1..29: mol_type = other DNA
catgctaggtgacagcattgagcagggct

SEQ ID NO: 342 - DNA, 23 bp, synthetic construct
  source 1..23: mol_type = other DNA
tggctaacgcctgtgatcccagc

SEQ ID NO: 343 - DNA, 31 bp, synthetic construct
  source 1..31: mol_type = other DNA
catggttcaagagcgatggctctcgaataaa

SEQ ID NO: 344 - DNA, 21 bp, synthetic construct
  source 1..21: mol_type = other DNA
ctcgctgcaacttccacccgc

SEQ ID NO: 345 - DNA, 26 bp, synthetic construct
  source 1..26: mol_type = other DNA
catggggcatcaccacaggctgagga

SEQ ID NO: 346 - DNA, 35 bp, synthetic construct
  source 1..35: mol_type = other DNA
tttcctgttatttctataagatgcttcctctgatt

SEQ ID NO: 347 - DNA, 31 bp, synthetic construct
  source 1..31: mol_type = other DNA
catggaggttgcattgagccaagatcatggc

SEQ ID NO: 348 - DNA, 26 bp, synthetic construct
  source 1..26: mol_type = other DNA
gagaatgcagcccagtaggtctcagc

SEQ ID NO: 349 - DNA, 31 bp, synthetic construct
  source 1..31: mol_type = other DNA
cgtatgaggctcgacaggtaccatatagcct

SEQ ID NO: 350 - DNA, 38 bp, synthetic construct
  source 1..38: mol_type = other DNA
cgtagactccaaccattggtataatctgaaagttgtct

SEQ ID NO: 351 - DNA, 25 bp, synthetic construct
  source 1..25: mol_type = other DNA
cgtactgcctgacaacggactgaaa

SEQ ID NO: 352 - DNA, 36 bp, synthetic construct
  source 1..36: mol_type = other DNA
cgtatcggctatttctaacagctcacatgtttacat

SEQ ID NO: 353 - DNA, 33 bp, synthetic construct
  source 1..33: mol_type = other DNA
cgtattccataagtgccagctggctgagaaata

SEQ ID NO: 354 - DNA, 33 bp, synthetic construct
  source 1..33: mol_type = other DNA
cgtatctgctcccaggttcaagtgattctcaag

SEQ ID NO: 355 - DNA, 26 bp, synthetic construct
  source 1..26: mol_type = other DNA
cgtatccgagagctcacagcttggct

SEQ ID NO: 356 - DNA, 30 bp, synthetic construct
  source 1..30: mol_type = other DNA
cgtagtttgatggaattcaccagtgtgttt

SEQ ID NO: 357 - DNA, 39 bp, synthetic construct
  source 1..39: mol_type = other DNA
cgtaatttagagagcctgtcacaaatgtccatatgagaa

SEQ ID NO: 358 - DNA, 27 bp, synthetic construct
  source 1..27: mol_type = other DNA
cgtagagaggagagagtgcagggtcaa

SEQ ID NO: 359 - DNA, 38 bp, synthetic construct
  source 1..38: mol_type = other DNA
cgtaacattattgttaacaagggttgcttctgggaaga

SEQ ID NO: 360 - DNA, 28 bp, synthetic construct
  source 1..28: mol_type = other DNA
cgtaccctggcacctagtgggtccatac

SEQ ID NO: 361 - DNA, 29 bp, synthetic construct
  source 1..29: mol_type = other DNA
cgtagcactccagagactctgtttctaaa

SEQ ID NO: 362 - DNA, 23 bp, synthetic construct
  source 1..23: mol_type = other DNA
cgtacaggcatggtagcgcgcct

SEQ ID NO: 363 - DNA, 32 bp, synthetic construct
  source 1..32: mol_type = other DNA
cgtaggaagcctcataatcatggtggaaggca

SEQ ID NO: 364 - DNA, 28 bp, synthetic construct
  source 1..28: mol_type = other DNA
cgtacatgtttaccgatgaagctgcaaa

SEQ ID NO: 365 - DNA, 37 bp, synthetic construct
  source 1..37: mol_type = other DNA
cgtaaggaagctcagctatacctgttactgataattt

SEQ ID NO: 366 - DNA, 27 bp, synthetic construct
  source 1..27: mol_type = other DNA
cgtacccatgcaggcctaggctagtgt

SEQ ID NO: 367 - DNA, 31 bp, synthetic construct
  source 1..31: mol_type = other DNA
cgtaacctaaaggacacatctcagtacattt

SEQ ID NO: 368 - DNA, 31 bp, synthetic construct
  source 1..31: mol_type = other DNA
cgtaactgaaggctctatgtgcaggaactcc

SEQ ID NO: 369 - DNA, 31 bp, synthetic construct
  source 1..31: mol_type = other DNA
cgtacctcccaaagtgctgggattataggca

SEQ ID NO: 370 - DNA, 27 bp, synthetic construct
  source 1..27: mol_type = other DNA
cgtaaactgttcttccaggcctgattt

SEQ ID NO: 371 - DNA, 28 bp, synthetic construct
  source 1..28: mol_type = other DNA
cgtacccatcctggcacgtactgctact

SEQ ID NO: 372 - DNA, 35 bp, synthetic construct
  source 1..35: mol_type = other DNA
cgtaaggcacactcaggctttacgattcttgttta

SEQ ID NO: 373 - DNA, 39 bp, synthetic construct
  source 1..39: mol_type = other DNA
cgtaattctccctgcaaattgtttgactctagaaatagc

SEQ ID NO: 374 - DNA, 30 bp, synthetic construct
  source 1..30: mol_type = other DNA
cgtagggtagtagcgatctgggttacccag

SEQ ID NO: 375 - DNA, 29 bp, synthetic construct
  source 1..29: mol_type = other DNA
cgtacattgacccaccggtcattcaggag

SEQ ID NO: 376 - DNA, 32 bp, synthetic construct
  source 1..32: mol_type = other DNA
cgtactgagggttatccaagtgacccattgca

SEQ ID NO: 377 - DNA, 26 bp, synthetic construct
  source 1..26: mol_type = other DNA
cgtacttgtacctggtgttcggttat

SEQ ID NO: 378 - DNA, 24 bp, synthetic construct
  source 1..24: mol_type = other DNA
cgtaccagcccaagatggatgaat

SEQ ID NO: 379 - DNA, 25 bp, synthetic construct
  source 1..25: mol_type = other DNA
cgtagcggtggaagtcattctgata

SEQ ID NO: 380 - DNA, 24 bp, synthetic construct
  source 1..24: mol_type = other DNA
cgtagagggccacagattggtatt

SEQ ID NO: 381 - DNA, 26 bp, synthetic construct
  source 1..26: mol_type = other DNA
cgtaactgaaaggtcagattcaggag

SEQ ID NO: 382 - DNA, 31 bp, synthetic construct
  source 1..31: mol_type = other DNA
gtactgaggctcgacaggtaccatatagcct

SEQ ID NO: 383 - DNA, 38 bp, synthetic construct
  source 1..38: mol_type = other DNA
gtacgactccaaccattggtataatctgaaagttgtct

SEQ ID NO: 384 - DNA, 25 bp, synthetic construct
  source 1..25: mol_type = other DNA
gtacctgcctgacaacggactgaaa

SEQ ID NO: 385 - DNA, 36 bp, synthetic construct
  source 1..36: mol_type = other DNA
gtactcggctatttctaacagctcacatgtttacat

SEQ ID NO: 386 - DNA, 33 bp, synthetic construct
  source 1..33: mol_type = other DNA
gtacttccataagtgccagctggctgagaaata

SEQ ID NO: 387 - DNA, 33 bp, synthetic construct
  source 1..33: mol_type = other DNA
gtactctgctcccaggttcaagtgattctcaag

SEQ ID NO: 388 - DNA, 26 bp, synthetic construct
  source 1..26: mol_type = other DNA
gtactccgagagctcacagcttggct

SEQ ID NO: 389 - DNA, 30 bp, synthetic construct
  source 1..30: mol_type = other DNA
gtacgtttgatggaattcaccagtgtgttt

SEQ ID NO: 390 - DNA, 39 bp, synthetic construct
  source 1..39: mol_type = other DNA
gtacatttagagagcctgtcacaaatgtccatatgagaa

SEQ ID NO: 391 - DNA, 27 bp, synthetic construct
  source 1..27: mol_type = other DNA
gtacgagaggagagagtgcagggtcaa

SEQ ID NO: 392 - DNA, 38 bp, synthetic construct
  source 1..38: mol_type = other DNA
gtacacattattgttaacaagggttgcttctgggaaga

SEQ ID NO: 393 - DNA, 28 bp, synthetic construct
  source 1..28: mol_type = other DNA
gtacccctggcacctagtgggtccatac

SEQ ID NO: 394 - DNA, 29 bp, synthetic construct
  source 1..29: mol_type = other DNA
gtacgcactccagagactctgtttctaaa

SEQ ID NO: 395 - DNA, 23 bp, synthetic construct
  source 1..23: mol_type = other DNA
gtaccaggcatggtagcgcgcct

SEQ ID NO: 396 - DNA, 32 bp, synthetic construct
  source 1..32: mol_type = other DNA
gtacggaagcctcataatcatggtggaaggca

SEQ ID NO: 397 - DNA, 28 bp, synthetic construct
  source 1..28: mol_type = other DNA
gtaccatgtttaccgatgaagctgcaaa

SEQ ID NO: 398 - DNA, 37 bp, synthetic construct
  source 1..37: mol_type = other DNA
gtacaggaagctcagctatacctgttactgataattt

SEQ ID NO: 399 - DNA, 27 bp, synthetic construct
  source 1..27: mol_type = other DNA
gtaccccatgcaggcctaggctagtgt

SEQ ID NO: 400 - DNA, 31 bp, synthetic construct
  source 1..31: mol_type = other DNA
gtacacctaaaggacacatctcagtacattt

SEQ ID NO: 401 - DNA, 31 bp, synthetic construct
  source 1..31: mol_type = other DNA
gtacactgaaggctctatgtgcaggaactcc

SEQ ID NO: 402 - DNA, 31 bp, synthetic construct
  source 1..31: mol_type = other DNA
gtaccctcccaaagtgctgggattataggca

SEQ ID NO: 403 - DNA, 27 bp, synthetic construct
  source 1..27: mol_type = other DNA
gtacaactgttcttccaggcctgattt

SEQ ID NO: 404 - DNA, 28 bp, synthetic construct
  source 1..28: mol_type = other DNA
gtaccccatcctggcacgtactgctact

SEQ ID NO: 405 - DNA, 35 bp, synthetic construct
  source 1..35: mol_type = other DNA
gtacaggcacactcaggctttacgattcttgttta

SEQ ID NO: 406 - DNA, 39 bp, synthetic construct
  source 1..39: mol_type = other DNA
gtacattctccctgcaaattgtttgactctagaaatagc

SEQ ID NO: 407 - DNA, 30 bp, synthetic construct
  source 1..30: mol_type = other DNA
gtacgggtagtagcgatctgggttacccag

SEQ ID NO: 408 - DNA, 29 bp, synthetic construct
  source 1..29: mol_type = other DNA
gtaccattgacccaccggtcattcaggag

SEQ ID NO: 409 - DNA, 32 bp, synthetic construct
  source 1..32: mol_type = other DNA
gtacctgagggttatccaagtgacccattgca

SEQ ID NO: 410 - DNA, 26 bp, synthetic construct
  source 1..26: mol_type = other DNA
gtaccttgtacctggtgttcggttat

SEQ ID NO: 411 - DNA, 24 bp, synthetic construct
  source 1..24: mol_type = other DNA
gtacccagcccaagatggatgaat

SEQ ID NO: 412 - DNA, 25 bp, synthetic construct
  source 1..25: mol_type = other DNA
gtacgcggtggaagtcattctgata

SEQ ID NO: 413 - DNA, 24 bp, synthetic construct
  source 1..24: mol_type = other DNA
gtacgagggccacagattggtatt

SEQ ID NO: 414 - DNA, 26 bp, synthetic construct
  source 1..26: mol_type = other DNA
gtacactgaaaggtcagattcaggag

SEQ ID NO: 415 - DNA, 31 bp, synthetic construct
  source 1..31: mol_type = other DNA
acgttgaggctcgacaggtaccatatagcct

SEQ ID NO: 416 - DNA, 38 bp, synthetic construct
  source 1..38: mol_type = other DNA
acgtgactccaaccattggtataatctgaaagttgtct

SEQ ID NO: 417 - DNA, 25 bp, synthetic construct
  source 1..25: mol_type = other DNA
acgtctgcctgacaacggactgaaa

SEQ ID NO: 418 - DNA, 36 bp, synthetic construct
  source 1..36: mol_type = other DNA
acgttcggctatttctaacagctcacatgtttacat

SEQ ID NO: 419 - DNA, 33 bp, synthetic construct
  source 1..33: mol_type = other DNA
acgtttccataagtgccagctggctgagaaata

SEQ ID NO: 420 - DNA, 33 bp, synthetic construct
  source 1..33: mol_type = other DNA
acgttctgctcccaggttcaagtgattctcaag

SEQ ID NO: 421 - RNA, 26 nt, synthetic construct
  source 1..26: mol_type = other RNA
acgttccgagagctcacagcttggct

SEQ ID NO: 422 - DNA, 30 bp, synthetic construct
  source 1..30: mol_type = other DNA
acgtgtttgatggaattcaccagtgtgttt

SEQ ID NO: 423 - DNA, 39 bp, synthetic construct
  source 1..39: mol_type = other DNA
acgtatttagagagcctgtcacaaatgtccatatgagaa

SEQ ID NO: 424 - DNA, 27 bp, synthetic construct
  source 1..27: mol_type = other DNA
acgtgagaggagagagtgcagggtcaa

SEQ ID NO: 425 - DNA, 38 bp, synthetic construct
  source 1..38: mol_type = other DNA
acgtacattattgttaacaagggttgcttctgggaaga

SEQ ID NO: 426 - DNA, 28 bp, synthetic construct
  source 1..28: mol_type = other DNA
acgtccctggcacctagtgggtccatac

SEQ ID NO: 427 - DNA, 29 bp, synthetic construct
  source 1..29: mol_type = other DNA
acgtgcactccagagactctgtttctaaa

SEQ ID NO: 428 - DNA, 23 bp, synthetic construct
  source 1..23: mol_type = other DNA
acgtcaggcatggtagcgcgcct

SEQ ID NO: 429 - DNA, 32 bp, synthetic construct
  source 1..32: mol_type = other DNA
acgtggaagcctcataatcatggtggaaggca

SEQ ID NO: 430 - DNA, 28 bp, synthetic construct
  source 1..28: mol_type = other DNA
acgtcatgtttaccgatgaagctgcaaa

SEQ ID NO: 431 - DNA, 37 bp, synthetic construct
  source 1..37: mol_type = other DNA
acgtaggaagctcagctatacctgttactgataattt

SEQ ID NO: 432 - DNA, 27 bp, synthetic construct
  source 1..27: mol_type = other DNA
acgtcccatgcaggcctaggctagtgt

SEQ ID NO: 433 - DNA, 31 bp, synthetic construct
  source 1..31: mol_type = other DNA
acgtacctaaaggacacatctcagtacattt

SEQ ID NO: 434 - DNA, 31 bp, synthetic construct
  source 1..31: mol_type = other DNA
acgtactgaaggctctatgtgcaggaactcc

SEQ ID NO: 435 - DNA, 31 bp, synthetic construct
  source 1..31: mol_type = other DNA
acgtcctcccaaagtgctgggattataggca

SEQ ID NO: 436 - DNA, 27 bp, synthetic construct
  source 1..27: mol_type = other DNA
acgtaactgttcttccaggcctgattt

SEQ ID NO: 437 - DNA, 28 bp, synthetic construct
  source 1..28: mol_type = other DNA
acgtcccatcctggcacgtactgctact

SEQ ID NO: 438 - DNA, 35 bp, synthetic construct
  source 1..35: mol_type = other DNA
acgtaggcacactcaggctttacgattcttgttta

SEQ ID NO: 439 - DNA, 39 bp, synthetic construct
  source 1..39: mol_type = other DNA
acgtattctccctgcaaattgtttgactctagaaatagc

SEQ ID NO: 440 - DNA, 30 bp, synthetic construct
  source 1..30: mol_type = other DNA
acgtgggtagtagcgatctgggttacccag

SEQ ID NO: 441 - DNA, 29 bp, synthetic construct
  source 1..29: mol_type = other DNA
acgtcattgacccaccggtcattcaggag

SEQ ID NO: 442 - DNA, 32 bp, synthetic construct
  source 1..32: mol_type = other DNA
acgtctgagggttatccaagtgacccattgca

SEQ ID NO: 443 - DNA, 26 bp, synthetic construct
  source 1..26: mol_type = other DNA
acgtcttgtacctggtgttcggttat

SEQ ID NO: 444 - DNA, 24 bp, synthetic construct
  source 1..24: mol_type = other DNA
acgtccagcccaagatggatgaat

SEQ ID NO: 445 - DNA, 25 bp, synthetic construct
  source 1..25: mol_type = other DNA
acgtgcggtggaagtcattctgata

SEQ ID NO: 446 - DNA, 24 bp, synthetic construct
  source 1..24: mol_type = other DNA
acgtgagggccacagattggtatt

SEQ ID NO: 447 - DNA, 26 bp, synthetic construct
  source 1..26: mol_type = other DNA
acgtactgaaaggtcagattcaggag

SEQ ID NO: 448 - DNA, 36 bp, synthetic construct
  source 1..36: mol_type = other DNA
catgttcatcacacacatgctaacacaaatccagtc

SEQ ID NO: 449 - DNA, 32 bp, synthetic construct
  source 1..32: mol_type = other DNA
cagaatccatgctcttaaccattatgctctgc

SEQ ID NO: 450 - DNA, 21 bp, synthetic construct
  source 1..21: mol_type = other DNA
catggaccgccggtgagccgg

SEQ ID NO: 451 - DNA, 28 bp, synthetic construct
  source 1..28: mol_type = other DNA
gaatgtactttgcaagtgagaatgacat

SEQ ID NO: 452 - DNA, 36 bp, synthetic construct
  source 1..36: mol_type = other DNA
catgtcatcctggacattagctacctctgtgattga

SEQ ID NO: 453 - DNA, 34 bp, synthetic construct
  source 1..34: mol_type = other DNA
acccagagtaggcattaatcattccagacatttc

SEQ ID NO: 454 - DNA, 26 bp, synthetic construct
  source 1..26: mol_type = other DNA
catggcacttgcctccaccttcccag

SEQ ID NO: 455 - DNA, 28 bp, synthetic construct
  source 1..28: mol_type = other DNA
ggtgaaactacttaatgggtgcagggtg

SEQ ID NO: 456 - DNA, 27 bp, synthetic construct
  source 1..27: mol_type = other DNA
catgccagggaccctagcaaacgagct

SEQ ID NO: 457 - DNA, 27 bp, synthetic construct
  source 1..27: mol_type = other DNA
tgaccctggtcagtattcctagggtcc

SEQ ID NO: 458 - DNA, 36 bp, synthetic construct
  source 1..36: mol_type = other DNA
catgccatatttataccagcctggctctaggagagt

SEQ ID NO: 459 - DNA, 29 bp, synthetic construct
  source 1..29: mol_type = other DNA
tcatagcaagcaatgacaatgccaataaa

SEQ ID NO: 460 - DNA, 34 bp, synthetic construct
  source 1..34: mol_type = other DNA
catgtctcccacactccagatccaaagactactt

SEQ ID NO: 461 - DNA, 34 bp, synthetic construct
  source 1..34: mol_type = other DNA
tgtagatacagttattctttgttactggttgaat

SEQ ID NO: 462 - DNA, 26 bp, synthetic construct
  source 1..26: mol_type = other DNA
catgcgtgcacgcgcatgcaaatagg

SEQ ID NO: 463 - DNA, 34 bp, synthetic construct
  source 1..34: mol_type = other DNA
caaataagcacataagataaagatccagctattt

SEQ ID NO: 464 - DNA, 36 bp, synthetic construct
  source 1..36: mol_type = other DNA
catgccctgaacatttcaccatgaaatttgtgagct

SEQ ID NO: 465 - DNA, 25 bp, synthetic construct
  source 1..25: mol_type = other DNA
gcctaagcctggaacctaggaggtg

SEQ ID NO: 466 - DNA, 29 bp, synthetic construct
  source 1..29: mol_type = other DNA
catggacggagtttcactctttcgcccag

SEQ ID NO: 467 - DNA, 22 bp, synthetic construct
  source 1..22: mol_type = other DNA
agaggtggcaatgggccgagat

SEQ ID NO: 468 - DNA, 33 bp, synthetic construct
  source 1..33: mol_type = other DNA
catgaaagtctcaagtagtcggcatatctgttt

SEQ ID NO: 469 - DNA, 25 bp, synthetic construct
  source 1..25: mol_type = other DNA
actgctggctggaaacagaggctaa

SEQ ID NO: 470 - DNA, 37 bp, synthetic construct
  source 1..37: mol_type = other DNA
catggtgataatttaaggcagtttctcatctgccttc

SEQ ID NO: 471 - DNA, 30 bp, synthetic construct
  source 1..30: mol_type = other DNA
catgcacttgtagttccaggtactagggag

SEQ ID NO: 472 - DNA, 30 bp, synthetic construct
  source 1..30: mol_type = other DNA
catgaaattagccggacatggtggtgcatg

SEQ ID NO: 473 - DNA, 21 bp, synthetic construct
  source 1..21: mol_type = other DNA
gtgcgcgcctgtaatcccagc

SEQ ID NO: 474 - DNA, 39 bp, synthetic construct
  source 1..39: mol_type = other DNA
catgaaattcccaagtcactttgttcagaaatatttcag

SEQ ID NO: 475 - DNA, 29 bp, synthetic construct
  source 1..29: mol_type = other DNA
aaagggactccaagtattgtacgatgaaa

SEQ ID NO: 476 - DNA, 38 bp, synthetic construct
  source 1..38: mol_type = other DNA
catgtccttagactttccttgctttcagtaaccttgag

SEQ ID NO: 477 - DNA, 26 bp, synthetic construct
  source 1..26: mol_type = other DNA
cactgagaggcctagaagcagtgacc

SEQ ID NO: 478 - DNA, 39 bp, synthetic construct
  source 1..39: mol_type = other DNA
catgtttagttagattgtcttcttattgttgagttacag

SEQ ID NO: 479 - DNA, 28 bp, synthetic construct
  source 1..28: mol_type = other DNA
ccacaaagtgcaaagtttcatgcaactt

SEQ ID NO: 480 - DNA, 38 bp, synthetic construct
  source 1..38: mol_type = other DNA
catgaattacatttaatgcctgttgatgagggctcaag

SEQ ID NO: 481 - DNA, 35 bp, synthetic construct
  source 1..35: mol_type = other DNA
gttctatccattaagatcatgctcactaatttaga

SEQ ID NO: 482 - DNA, 28 bp, synthetic construct
  source 1..28: mol_type = other DNA
catgtttagtagcatggccatgtgcttt

SEQ ID NO: 483 - DNA, 27 bp, synthetic construct
  source 1..27: mol_type = other DNA
gaccagcctaggcaacatagtgagacc

SEQ ID NO: 484 - DNA, 23 bp, synthetic construct
  source 1..23: mol_type = other DNA
catggccaaggcccaccctgcag

SEQ ID NO: 485 - DNA, 22 bp, synthetic construct
  source 1..22: mol_type = other DNA
caggattccgtaagccaggttt

SEQ ID NO: 486 - DNA, 32 bp, synthetic construct
  source 1..32: mol_type = other DNA
catgcaaatcaaatcccagctgagccactctg

SEQ ID NO: 487 - DNA, 24 bp, synthetic construct
  source 1..24: mol_type = other DNA
ttctcatcttcccagctgacattt

SEQ ID NO: 488 - DNA, 34 bp, synthetic construct
  source 1..34: mol_type = other DNA
catgaatccacccacctagaattctgtactcagc

SEQ ID NO: 489 - DNA, 31 bp, synthetic construct
  source 1..31: mol_type = other DNA
gactcaatttatgttgtttccattccagttt

SEQ ID NO: 490 - DNA, 24 bp, synthetic construct
  source 1..24: mol_type = other DNA
catgccaccacgcccagctaattt

SEQ ID NO: 491 - DNA, 31 bp, synthetic construct
  source 1..31: mol_type = other DNA
agtatgaagcccacctagattcattgtgagg

SEQ ID NO: 492 - DNA, 39 bp, synthetic construct
  source 1..39: mol_type = other DNA
catgatttatttacaactgtgattggacaagaagaattc

SEQ ID NO: 493 - DNA, 23 bp, synthetic construct
  source 1..23: mol_type = other DNA
cgatctcggctcactgcaacctc

SEQ ID NO: 494 - DNA, 37 bp, synthetic construct
  source 1..37: mol_type = other DNA
catgattatttatatgatcttcacctgtgcaattcct

SEQ ID NO: 495 - DNA, 29 bp, synthetic construct
  source 1..29: mol_type = other DNA
caggagacctctctgagtagttgacaatt

SEQ ID NO: 496 - DNA, 36 bp, synthetic construct
  source 1..36: mol_type = other DNA
catgatctctcaagtgagaggattcttccttcctga

SEQ ID NO: 497 - DNA, 31 bp, synthetic construct
  source 1..31: mol_type = other DNA
gacttcagaagctagttactgaaggaatact

SEQ ID NO: 498 - DNA, 39 bp, synthetic construct
  source 1..39: mol_type = other DNA
catgaataagtctctcagtcattatattagcttctaaca

SEQ ID NO: 499 - DNA, 28 bp, synthetic construct
  source 1..28: mol_type = other DNA
atgtaatgacggacttagtggcaataaa

SEQ ID NO: 500 - DNA, 35 bp, synthetic construct
  source 1..35: mol_type = other DNA
catggggaagaggtcaggatttgcacctatatctg

SEQ ID NO: 501 - DNA, 24 bp, synthetic construct
  source 1..24: mol_type = other DNA
aatcctcccagcactttgggaggc

SEQ ID NO: 502 - DNA, 30 bp, synthetic construct
  source 1..30: mol_type = other DNA
catgctcaggactgaagggtttcccaggac

SEQ ID NO: 503 - DNA, 22 bp, synthetic construct
  source 1..22: mol_type = other DNA
gctattcgggaggctgaggcag

SEQ ID NO: 504 - DNA, 37 bp, synthetic construct
  source 1..37: mol_type = other DNA
catgatgtttagttatgccctatgcagatgtcttggt

SEQ ID NO: 505 - DNA, 33 bp, synthetic construct
  source 1..33: mol_type = other DNA
tatgtctactctgtagcatttggtggttcatcc

SEQ ID NO: 506 - DNA, 22 bp, synthetic construct
  source 1..22: mol_type = other DNA
catggggacggtacggtgcgcg

SEQ ID NO: 507 - DNA, 35 bp, synthetic construct
  source 1..35: mol_type = other DNA
cagataatcagcattaataatatgtccagcatgac

SEQ ID NO: 508 - DNA, 39 bp, synthetic construct
  source 1..39: mol_type = other DNA
catgacataaagactttgctgatgatatattaacaaatg

SEQ ID NO: 509 - DNA, 33 bp, synthetic construct
  source 1..33: mol_type = other DNA
caatagtgggacaggcatagcatagacattctc

SEQ ID NO: 510 - DNA, 28 bp, synthetic construct
  source 1..28: mol_type = other DNA
catgacggcatatcctgctcaccgactt

SEQ ID NO: 511 - DNA, 29 bp, synthetic construct
  source 1..29: mol_type = other DNA
atgggcactattgctttctaaatgcacaa

SEQ ID NO: 512 - DNA, 31 bp, synthetic construct
  source 1..31: mol_type = other DNA
catgtttcctgtgctgaagtgaccctttggt

SEQ ID NO: 513 - DNA, 29 bp, synthetic construct
  source 1..29: mol_type = other DNA
ggaagttcatgcctaagaaacatgtagca

SEQ ID NO: 514 - DNA, 36 bp, synthetic construct
  source 1..36: mol_type = other DNA
catgttaggtcttagatttaagttcctgatccattt

SEQ ID NO: 515 - DNA, 22 bp, synthetic construct
  source 1..22: mol_type = other DNA
tctcggctcactgcaagctccg

(19) United States
(12) Patent Application Publication
(10) Pub. No.: US 2026/0286436 A1
(43) Pub. Date: Sep. 24, 2026

(54) METHODS FOR IDENTIFYING CROSS-CONTAMINATION AMONG SEQUENTIAL NUCLEIC ACID AMPLIFICATION REACTIONS

(71) Applicant: Wisconsin Alumni Research Foundation, Madison, WI (US)

(72) Inventors: Muhammed Murtaza, Middleton, WI (US); Amanda Schussman, Madison, WI (US); Bradon McDonald, Madison, WI (US); Kirsten Dennison, Madison, WI (US)

(73) Assignee: Wisconsin Alumni Research Foundation, Madison, WI (US)

(21) Appl. No.: 19/573,120

(22) Filed: Mar. 20, 2026

Related U.S. Application Data
(60) Provisional application No. 63/774,954, filed on Mar. 20, 2025.

(51) Int. Cl. C12Q 1/6876; C12Q 1/6848; C12Q 1/6869; C40B 40/06; C40B 50/06; C40B 70/00

(52) U.S. Cl. CPC C12Q 1/6876; C12Q 1/6848; C12Q 1/6869; C12Q 2600/16; C40B 40/06; C40B 50/06; C40B 70/00

(57) ABSTRACT

The invention is directed to methods for identifying cross-contamination among sequential nucleic acid amplification reactions, such as with the use of molecularly-tagged primers in the amplification reactions.

### CROSS-REFERENCE TO RELATED APPLICATIONS

Priority is hereby claimed to U.S. Provisional Application 63/774,954, filed Mar. 20, 2025, which is incorporated herein by reference in its entirety.

### STATEMENT REGARDING FEDERALLY SPONSORED RESEARCH

This invention was made with government support under CA243078 and CA234306 awarded by the National Institutes of Health. The government has certain rights in the invention.

### SEQUENCE LISTING

The instant application contains a Sequence Listing which has been submitted in XML format and is hereby incorporated by reference in its entirety. The XML copy, created on Mar. 19, 2026, is named USPTO-260320-09824664-P240138US02-APP-SEQ LIST and is 459,815 bytes in size.

### FIELD OF THE INVENTION

The invention is directed to methods for identifying cross-contamination among sequential nucleic acid amplification reactions, such as with the use of molecularly-tagged primers in the amplification reactions.

### BACKGROUND

Cross-contamination between diagnostic assays based on PCR has been a long-recognized challenge in molecular diagnostics. PCR relies on exponential amplification of targeted genomic loci but the sheer molecular abundance (quantity and concentration) of the resulting amplicons can linger in laboratory surfaces and as aerosol. A key strategy to overcome such cross-contamination is a dedicated workflow that separate pre-PCR and post-PCR steps of an assay. This works remarkably well except when an assay involves multiple PCR steps, such that after some initial exponential amplification, the assay can no longer be performed in the template-free/amplicon-free labs and later PCR setups must be completed in a post-PCR environment. When the final readout is performed using next-generation sequencing, such cross-contamination before sample barcoding can make it a challenge to interpret downstream results. Tools and methods that permit distinguishing whether a detected amplicon was truly present in template DNA or if it was lingering cross-contamination from a previous experiment are needed.

### SUMMARY OF THE INVENTION

One aspect of the invention is directed to methods of analyzing nucleic acids. The methods can comprise sequentially amplifying template nucleic acid with each primer set within the group. In preferred versions, the group of primer sets comprises two or more primer sets, each primer set comprises set-identity primer pairs, each set-identity primer pair consists of set-identity primers consisting of a first set-identity primer and a second set-identity primer, each set-identity primer comprises a template-annealing segment on a 3′ end of the set-identity primer, each set-identity primer comprises a set-identity segment on a 5′ end of the set-identity primer, a combination of the set-identity segment on the first set-identity primer and the set-identity segment on the second set-identity primer in each set-identity primer pair defines a set-identity signature, and each of the set-identity signatures is defined only by set-identity primer pairs in one and only one of primer sets within the group.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1D. Possible source of contamination from experiment to experiment in an exemplary workflow (Structural Variant Enrichment and Normalization (SVEN) workflow (WO 2025/006244 A1)). FIG. 1A. Step 1, multiplexed preamplification for tumor structural variant (SV) enrichment. FIG. 1B. Step 2, library preparation and adapter ligation to amplicons. FIG. 1C. Step 3, quantification of individual libraries and pool. FIG. 1D. Step 4, amplicon sequencing and analysis. Step 1 (PCR product generation) is the likely culprit for run-to-run carryover. Contaminating carry-over DNA molecules can contaminate Step 1 (PCR product generation) or Step 2 (amplicon library construction) in subsequent runs. Amplicons that linger from Step 1 in a first run, for example, could end up serving as template in Step 1 and/or Step 2 subsequent run, thereby contaminating the subsequent experiments and leading to erroneous results.

FIGS. 2A and 2B. Target contamination in no-template controls (NTCs) by lab space with eight replicates per lab space. The template-free lab space (circle) is where reaction master mixes are created, and therefore, no contaminating molecules are anticipated, either control or structural variant (SV). The extraction lab space (square) is where DNA is extracted, prepared, or otherwise added to various molecular assays. Because this space is used for DNA extraction, some level of control target amplification and potentially SV target amplification is anticipated due to extraction/preparation of tumor templates in this space. These two spaces should have low-level contamination as these are pre-amplification spaces. Conversely, the main lab space (triangle) is a post-amplification space, and a higher degree of both control and SV contaminating amplicons is anticipated. FIG. 2A. Control target contamination. Contamination was noted in all lab spaces with the most persistent contamination noted in the extraction lab space as expected. FIG. 2B. Structural variant (SV) target contamination. NTCs demonstrated increasing SV contamination as more DNA or amplicons are expected to be present with the template-free lab having the least contamination, the extraction lab having a middle amount of contamination, and the main lab space having extremely high levels of contamination. Y-axes are log-scale; replicates without any targets identified are labelled as “Not Detected”.

FIGS. 3A-3C. Tagged primer approach. FIG. 3A. 4-bp tags were added to the 5′ end of only the forward primers. Reverse primer and 3′ ends remained untagged to prevent color-balance concerns during paired-end sequencing. FIGS. 3B and 3C. Use of tags as a prep-to-prep contamination control, showing a first run on a template with a Tag1 forward primer (Forward_Tag1) (FIG. 3B) and a second run on the same template with a Tag2 forward primer (Forward_Tag2) (FIG. 3C), in which Tag1 amplicons from the first run are carried over as contamination into the second run and filtered out of the analysis. 5′ addition does not significantly affect primer performance. Tags permit informatically filtering lingering contamination from previous runs with the same patient. The system can quantify the level of contamination, track down sources and timing of contamination, and provide longitudinal tracking for quality control at scale. The method is broadly applicable where post-amplification contamination is a concern, particularly with low-abundance targets.

FIGS. 4A and 4B. Longitudinal use of tagged system in both primary patient samples (FIG. 4A) and in model systems (FIG. 4B). FIG. 4A. SV target contamination levels when the tag remains the same between runs in patient buffy coat samples (normal, n=2). Each patient's identifier is on the x-axis. For each sample analyzed, the points on the left show results from a first run (EXP_1, circle) and the points on the right show results from a second, subsequent run (EXP_2, square). For four patient panels, there was a demonstrable increase in contamination between EXP_1 and EXP_2 using the same tag, implicating run-to-run carryover. For the remaining panels, SV detection was either decreased in the subsequent run (two panels) or both runs had no SVs detected (one panel). FIG. 4B. SV target contamination levels when the tag is switched between runs in a cell line model system that has been used extensively for development. A first run (EXP_1, n=4, circle) used Tag2 and showed a low-level of contamination. Contamination was eliminated when switching to Tag3 in a second run (EXP_2, n=8, square) and to Tag1 in a third run (EXP_3, n=3, triangle), demonstrating the utility of this tagged-primer approach.

### DETAILED DESCRIPTION OF THE INVENTION

One aspect of the invention is directed to methods of analyzing nucleic acid with a group of primer sets.

The methods can comprise sequentially amplifying template nucleic acid with each primer set within the group. “Sequentially amplifying template nucleic acid with each primer set within the group” as used herein means that the primers in each individual primer set are all employed together in at least one amplification reaction that is separate from the amplification reactions employing the other primer sets and, further, that the primer sets in the group are employed in a series of amplification reactions such that no single primer set in the group is re-employed after another primer set in the group until each primer set in the group is employed in at least one amplification reaction. “Amplification reaction” as used herein refers to the replication of at least a portion of a nucleic acid template with a nucleic acid primer. “Replication” refers to the copying of a nucleic acid sequence contained in a nucleic acid template and can encompass copying the sequence from a DNA template to a DNA product, an RNA template to a DNA product, or a DNA template to an RNA product. “Primer” refers to a nucleic acid that binds to the nucleic acid template and serves as a starting point for polymerization from same. The primers of the invention can comprise DNA, RNA, or a combination of the two.

In some versions, each sequential amplification reaction is conducted in an enclosed reaction mixture and produces an enclosed product mixture. “Enclosed mixture” and variants thereof such as “enclosed amplification mixture” and “enclosed product mixture” refer to a mixture in a given phase that is completely surrounded by an enclosure of one or more immiscible phases. In some versions, the mixture is a liquid, aqueous phase. In some versions, the enclosure is entirely a solid phase. In some versions, the enclosure is entirely a non-water-immiscible (e.g., oil) liquid phase. In some versions, the enclosure is a combination of a solid phase and a non-water-immiscible liquid phase.

In some versions, the methods comprise exposing one or more of the enclosed product mixtures. An enclosed product mixture is understood herein to be exposed when the enclosure of the product mixture is breached such that the product mixture is exposed to any substance outside the enclosure, such as surrounding gas (e.g., air), a pipet tip, a needle, a molecule introduced from outside the enclosure, etc. In some versions, each product mixture is sequentially exposed. In some versions, the product mixtures are sequentially exposed in the order in which they were generated.

Each primer set can comprise one or more primer pairs. “Primer pair” as used herein refers to a pair of primers designed to amplify a target segment of a template nucleic acid. The target segment may or may not be present in any given amplification reaction.

In the present invention, the primer pairs in each primer set comprises or consists of set-identity primer pairs. Each set-identity primer pair consists of set-identity primers consisting of a first set-identity primer and a second set-identity primer. In some versions, the first set-identity primer can be considered to be a forward primer, and the second set-identity primer can be considered to be a reverse primer. In some versions, the second set-identity primer can be considered to be a forward primer, and the first set-identity primer can be considered to be a reverse primer. “Forward primer,” as understood in the art, refers to a primer that binds to an antisense strand of a nucleic acid template, and “reverse primer,” as understood in the art, refers to a primer that binds to a sense strand of a nucleic acid template.

In some versions, each set-identity primer comprises a template-annealing segment on a 3′ end thereof. “3′ end” of a primer refers to a 3′ terminal base and one or more contiguous bases upstream (e.g., 5′) thereof “Template-annealing segment” refers to a segment of the primer designed to anneal to target site on a template nucleic acid. The target site may or may not be present in any given amplification reaction. The template-annealing segment may be completely or partially complementary to the target site. However, in order to prime a polymerization reaction, at least the 3′ terminal base is typically thought to be required to be complementary (form a base pair) with template nucleic acid.

In various versions of the invention, the template-annealing segment on any given set-identity primer can be at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, at least 18 bases, at least 19 bases, at least 20 bases, at least 21 bases, at least 22 bases, at least 23 bases, at least 24 bases, at least 25 bases, at least 26 bases, at least 27 bases, at least 28 bases, at least 29 bases, or at least 30 bases.

In various versions of the invention, the template-annealing segment on each set-identity primer in each primer set can be at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, at least 18 bases, at least 19 bases, at least 20 bases, at least 21 bases, at least 22 bases, at least 23 bases, at least 24 bases, at least 25 bases, at least 26 bases, at least 27 bases, at least 28 bases, at least 29 bases, or at least 30 bases.

In various versions of the invention, the template-annealing segment on any given set-identity primer can be up to 6 bases, up to 7 bases, up to 8 bases, up to 9 bases, up to 10 bases, up to 11 bases, up to 12 bases, up to 13 bases, up to 14 bases, up to 15 bases, up to 16 bases, up to 17 bases, up to 18 bases, up to 19 bases, up to 20 bases, up to 21 bases, up to 22 bases, up to 23 bases, up to 24 bases, up to 25 bases, up to 26 bases, up to 27 bases, up to 28 bases, up to 29 bases, up to 30 bases, up to 31 bases, up to 32 bases, up to 33 bases, up to 34 bases, up to 35 bases, up to 36 bases, up to 37 bases, up to 38 bases, up to 39 bases, up to 40 bases, up to 41 bases, up to 42 bases, up to 43 bases, up to 44 bases, up to 45 bases, up to 46 bases, up to 47 bases, up to 48 bases, up to 49 bases, up to 50 bases, up to 51 bases, up to 52 bases, up to 53 bases, up to 54 bases, up to 55 bases, up to 56 bases, up to 57 bases, up to 58 bases, up to 59 bases, or up to 60 bases or more in length.

In various versions of the invention, the template-annealing segment on each set-identity primer in each primer set can be up to 6 bases, up to 7 bases, up to 8 bases, up to 9 bases, up to 10 bases, up to 11 bases, up to 12 bases, up to 13 bases, up to 14 bases, up to 15 bases, up to 16 bases, up to 17 bases, up to 18 bases, up to 19 bases, up to 20 bases, up to 21 bases, up to 22 bases, up to 23 bases, up to 24 bases, up to 25 bases, up to 26 bases, up to 27 bases, up to 28 bases, up to 29 bases, up to 30 bases, up to 31 bases, up to 32 bases, up to 33 bases, up to 34 bases, up to 35 bases, up to 36 bases, up to 37 bases, up to 38 bases, up to 39 bases, up to 40 bases, up to 41 bases, up to 42 bases, up to 43 bases, up to 44 bases, up to 45 bases, up to 46 bases, up to 47 bases, up to 48 bases, up to 49 bases, up to 50 bases, up to 51 bases, up to 52 bases, up to 53 bases, up to 54 bases, up to 55 bases, up to 56 bases, up to 57 bases, up to 58 bases, up to 59 bases, or up to 60 bases or more in length.

As used herein, “base length” refers to the number of bases contained by a particular segment, such as a set-identity segment, and can include 0 bases (in a null limiting case) to any positive number of bases. “Base sequence” as used herein refers to a sequence of 2 or more bases as is understood in the art, except that the term as used herein, unless the context dictates otherwise, also encompasses the identity of a single base in cases where a base length of only 1 is present and a null sequence in a limiting case in which a base length of 0 is present. It is understood that two elements consisting of the same base sequence necessarily have the same base length. It is also understood that two elements consisting of different base lengths are necessarily defined by different base sequences.

In various versions of the invention, the template-annealing segment of each of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or each of the first set-identity primers in each primer set has a base length and base sequence identical to a base length and base sequence of the template-annealing segment of at least one of the first set-identity primers in each of the other sets.

In various versions of the invention, the template-annealing segment of each of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or each of the second set-identity primers in each primer set has a base length and base sequence identical to a base length and base sequence of the template-annealing segment of at least one of the second set-identity primers in each of the other sets.

In some versions, each set-identity primer comprises a set-identity segment on a 5′ end thereof. Except in a null limiting case (discussed below) in which a particular set-identity segment has a base length of 0, the “5′ end” of a primer refers to a 5′ terminal base and one or more contiguous bases downstream (i.e., 3′) thereof. In a null limiting case in which a particular set-identity segment has a base length of 0, “5′ end” refers to the 5′ terminus of the set-identity primer. “Set-identity segment” refers to a detectable characteristic on the 5′ end of each set-identity primer that identifies each set-identity primer pair as a member of a given set. Exemplary detectable characteristics include labeled bases, modified bases, base lengths, or base sequences, and, in a null limiting case, a lack of any or all of any one or more of the foregoing. In preferred versions of the invention, the detectable characteristic of each set-identity segment comprises a base length, a base sequence, or a combination thereof.

The set-identity segments on each set-identity primer pair can define the primer pair as a member of a given set by defining a set-identity signature. A set-identity signature is the combination of detectable characteristics on the first and second set-identity primers of a given set-identity primer pair. The set-identity signatures can identify a set-identity primer pair as a member of a primer set by being defined by the set-identity primer pairs in only one of the sets. In other words, multiple different set-identity signatures can be defined in a given primer set, but multiple primer sets would not define the same set-identity signature.

In various versions of the invention, all the set-identity primer pairs within a given primer set together define no more than 20, no more than 15, no more than 10, no more than 5, no more than 4 no more than 3, no more than 2, or no more than 1 different set-identity signatures. In various versions of the invention, all the set-identity primer pairs within each primer set together define no more than 20, no more than 15, no more than 10, no more than 5, no more than 4 no more than 3, no more than 2, no more than 1 different set-identity signatures. In some versions, all the set-identity primer pairs within a given primer set define the same set-identity signature. In some versions, all the set-identity primer pairs within each primer set define the same set-identity signature.

It should be noted that only one detectable difference in the combination of the first and second set-identity primers in a primer pair are required to distinguish that primer pair from a primer pair of a different set. This permits a variety of different configurations among the detectable characteristics in the set-identity segments of each set-identity primer pair. For example, a primer pair in a first set with a first set-identity primer having a set-identity segment of 0 bases and the second set-identity primer having a set-identity segment of 0 bases would distinguish from a primer pair in a second set with a first set-identity primer having a set-identity segment of 1 or more bases and the second set-identity primer having a set-identity segment of 0 bases. In another example, a primer pair in a first set with a first set-identity primer having a set-identity segment of 1 or more bases and the second set-identity primer having a set-identity segment of 0 bases would distinguish from a primer pair in a second set with a first set-identity primer having a set-identity segment of 0 bases and the second set-identity primer having a set-identity segment of 1 or more bases. In another example, a primer pair in a first set with a first set-identity primer having a set-identity segment of 1 base and the second set-identity primer having a set-identity segment of 0 bases would distinguish from a primer pair in a second set with a first set-identity primer having a set-identity segment of 2 or more bases and the second set-identity primer having a set-identity segment of 0 bases. In another example, a primer pair in a first set with a first set-identity primer having a set-identity segment of 1 or more bases defining a first sequence and the second set-identity primer having a set-identity segment of 0 bases would distinguish from a primer pair in a second set with a first set-identity primer having a set-identity segment of 1 or more bases defining a second sequence and the second set-identity primer having a set-identity segment of 0 bases. In another example, a primer pair in a first set with a first set-identity primer having a set-identity segment of 1 or more bases defining a first sequence and the second set-identity primer having a set-identity segment of 1 or more bases defining a second sequence would distinguish from a primer pair in a second set with a first set-identity primer having a set-identity segment of 1 or more bases defining a third sequence and the second set-identity primer having a set-identity segment of 1 or more bases defining the second sequence. Further examples would be evident to a practitioner in the art. As evident from above, one or more primers in different sets may comprise a null (base length of 0) set-identity segment, provided that at least one set-identity primer in each set-identity primer pair comprises a detectable characteristic that distinguishes from the set-identity primer pairs of other sets.

In various versions of the invention, the set-identity segment on any given set-identity primer can be at least 0 bases, at least 1 base, at least 2 bases, at least 3 bases, at least 4 bases, at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, at least 18 bases, at least 19 bases, at least 20 bases, at least 21 bases, at least 22 bases, at least 23 bases, at least 24 bases, at least 25 bases, at least 26 bases, at least 27 bases, at least 28 bases, at least 29 bases, or at least 30 bases.

In various versions of the invention, the set-identity segment on each set-identity primer can be at least 0 bases, at least 1 base, at least 2 bases, at least 3 bases, at least 4 bases, at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, at least 18 bases, at least 19 bases, at least 20 bases, at least 21 bases, at least 22 bases, at least 23 bases, at least 24 bases, at least 25 bases, at least 26 bases, at least 27 bases, at least 28 bases, at least 29 bases, or at least 30 bases.

In various versions of the invention, the set-identity segment on any given set-identity primer can be up to 1 base, up to 2 bases, up to 3 bases, up to 4 bases, up to 5 bases, up to 6 bases, up to 7 bases, up to 8 bases, up to 9 bases, up to 10 bases, up to 11 bases, up to 12 bases, up to 13 bases, up to 14 bases, up to 15 bases, up to 16 bases, up to 17 bases, up to 18 bases, up to 19 bases, up to 20 bases, up to 21 bases, up to 22 bases, up to 23 bases, up to 24 bases, up to 25 bases, up to 26 bases, up to 27 bases, up to 28 bases, up to 29 bases, up to 30 bases, up to 31 bases, up to 32 bases, up to 33 bases, up to 34 bases, up to 35 bases, up to 36 bases, up to 37 bases, up to 38 bases, up to 39 bases, up to 40 bases, up to 41 bases, up to 42 bases, up to 43 bases, up to 44 bases, up to 45 bases, up to 46 bases, up to 47 bases, up to 48 bases, up to 49 bases, up to 50 bases, up to 51 bases, up to 52 bases, up to 53 bases, up to 54 bases, up to 55 bases, up to 56 bases, up to 57 bases, up to 58 bases, up to 59 bases, or up to 60 bases or more in length.

In various versions of the invention, the set-identity segment on each set-identity primer can be up to 1 base, up to 2 bases, up to 3 bases, up to 4 bases, up to 5 bases, up to 6 bases, up to 7 bases, up to 8 bases, up to 9 bases, up to 10 bases, up to 11 bases, up to 12 bases, up to 13 bases, up to 14 bases, up to 15 bases, up to 16 bases, up to 17 bases, up to 18 bases, up to 19 bases, up to 20 bases, up to 21 bases, up to 22 bases, up to 23 bases, up to 24 bases, up to 25 bases, up to 26 bases, up to 27 bases, up to 28 bases, up to 29 bases, up to 30 bases, up to 31 bases, up to 32 bases, up to 33 bases, up to 34 bases, up to 35 bases, up to 36 bases, up to 37 bases, up to 38 bases, up to 39 bases, up to 40 bases, up to 41 bases, up to 42 bases, up to 43 bases, up to 44 bases, up to 45 bases, up to 46 bases, up to 47 bases, up to 48 bases, up to 49 bases, up to 50 bases, up to 51 bases, up to 52 bases, up to 53 bases, up to 54 bases, up to 55 bases, up to 56 bases, up to 57 bases, up to 58 bases, up to 59 bases, or up to 60 bases or more in length.

In various versions of the invention, each set-identity segment comprises from 0 to 10 bases, such as from 0 to 9 bases, from 0 to 8 bases, from 0 to 7 bases, from 0 to 6 bases, from 0 to 5 bases, from 0 to 4 bases, from 0 to 3 bases, from 0 to 2 bases, from 0 to 1 bases, from 1 to 9 bases, from 1 to 8 bases, from 1 to 7 bases, from 1 to 6 bases, from 1 to 5 bases, from 1 to 4 bases, from 1 to 3 bases, from 1 to 2 bases, from 2 to 6 bases, from 2 to 5 bases, or from 3 to 5 bases.

In some versions, each set-identity segment on each first set-identity primer in each primer set has a different base sequence than each set-identity segment on each first set-identity primer in each other set. In some versions, the set-identity segments on all the first set-identity primers in all the sets have the same base length. In some versions, set-identity segments on all the second set-identity primers in all the sets have the same base length. In some versions, set-identity segments on all the second set-identity primers in all the sets have the same base sequence. In some versions, set-identity segments on all the second set-identity primers in all the sets have the same base length and the same base sequence.

In some versions, one or more set-identity segments comprise a non-complimentary overhang. In some versions, the set-identity segment on each first set-identity primer comprises a non-complimentary overhang. “Non-complimentary overhang” refers to a set-identity segment in which at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% of the bases in the segment are non-complimentary (i.e., do not base pair) with a corresponding base on the template nucleic acid when the template-annealing segment is bound to the template nucleic acid.

In various versions of the invention, each primer set can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99 set-identity primer pairs. In various versions of the invention, each primer set can comprise up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35, up to 36, up to 37, up to 38, up to 39, up to 40, up to 41, up to 42, up to 43, up to 44, up to 45, up to 46, up to 47, up to 48, up to 49, up to 50, up to 51, up to 52, up to 53, up to 54, up to 55, up to 56, up to 57, up to 58, up to 59, up to 60, up to 61, up to 62, up to 63, up to 64, up to 65, up to 66, up to 67, up to 68, up to 69, up to 70, up to 71, up to 72, up to 73, up to 74, up to 75, up to 76, up to 77, up to 78, up to 79, up to 80, up to 81, up to 82, up to 83, up to 84, up to 85, up to 86, up to 87, up to 88, up to 89, up to 90, up to 91, up to 92, up to 93, up to 94, up to 95, up to 96, up to 97, up to 98, up to 99, up to 100, or more set-identity primer pairs. The counted set-identity primer pairs in such cases are understood to be different from each other. Set-identity primer pairs are considered herein to be different if at least one of the respective first set-identity primers or the respective second set-identity primers have at least one difference in base length or base sequence. Set-identity primer pairs are considered herein to be the same if the respective first set-identity primers have identical base lengths and base sequences and the respective second set-identity primers have identical base lengths and base sequences. Set-identity primers are considered herein to be different if the primers have at least one difference in base length or base sequence. Set-identity primers are considered herein to be the same if the primers have identical base lengths and base sequences.

In some versions of the invention, each primer set comprises multiple copies of each first set-identity primer and each second set-identity primer. Two molecules are considered herein to be “copies” of a primer when each molecule has an identical base length and base sequence.

In various versions of the invention, the set-identity primers in each primer set can be configured to amplify at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 different nonoverlapping target segments. In various versions of the invention, the set-identity primers in each primer set can be configured to amplify up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35, up to 36, up to 37, up to 38, up to 39, up to 40, up to 41, up to 42, up to 43, up to 44, up to 45, up to 46, up to 47, up to 48, up to 49, or up to 50 or more different nonoverlapping target segments. “Different nonoverlapping target segments” refers to target segments of target nucleic acid that have no overlapping bases.

In some versions, the set-identity primer pairs in each primer set are configured to amplify the same target segments.

In various versions of the invention, the group of primer sets can comprise 2 or more primer sets, 3 or more primer sets, 4 or more primer sets, 5 or more primer sets, 6 or more primer sets, 7 or more primer sets, 8 or more primer sets, 9 or more primer sets, 10 or more primer sets, 11 or more primer sets, 12 or more primer sets, 13 or more primer sets, 14 or more primer sets, 15 or more primer sets, 16 or more primer sets, 17 or more primer sets, 18 or more primer sets, 19 or more primer sets, or 20 or more primer sets. In various versions of the invention, the group of primer sets can comprise up to 2 primer sets, up to 3 primer sets, up to 4 primer sets, up to 5 primer sets, up to 6 primer sets, up to 7 primer sets, up to 8 primer sets, up to 9 primer sets, up to 10 primer sets, up to 11 primer sets, up to 12 primer sets, up to 13 primer sets, up to 14 primer sets, up to 15 primer sets, up to 16 primer sets, up to 17 primer sets, up to 18 primer sets, up to 19 primer sets, up to 20 primer sets, up to 25 primer sets, up to 30 primer set, up to 35 primer sets or more. The counted primer sets in such cases are understood to be different from each other. A first primer set is understood to be different from a second primer set if none of the set-identity signatures defined in the first primer set are defined in the second primer set.

In some versions, each sequential amplification comprises mixing the template nucleic acid and one of the primer sets, wherein the mixing for all the sequential amplifications is performed within a single enclosable mixing space. “Single enclosable mixing space” as used herein refers to a space suitable for the mixing steps that is enclosable with a barrier impassable to humans.

In some versions, the sequential amplifications can comprise generating a separate amplicon sample from each primer set. Each amplicon sample can comprise amplicons comprising one or more of the set-identity signatures. The sequences of the amplicons in each separate amplicon sample can then be determined. From the sequences, the set-identity signatures in the sequences of the amplicons in each of the amplicon samples can be identified. Sequences of the amplicons from any given amplicon sample that comprise a set-identity signature not defined by the primer set used to generate the amplicon sample can then be informatically filtered from further analysis.

In some versions, the sequence of the amplicons in each amplicon sample are determined. The sequence determination can include any of a number of steps, such as generating one or more sequencing libraries and sequencing the sequencing libraries in a nucleic acid sequencing instrument. “Sequencing library” as used herein refers to a collection of nucleic acid fragments suitable for a sequencing instrument, such as a next-generation sequencing instrument or long-read technologies such as Oxford Nanopore and PacBio, among others. Generating a sequencing library can comprise any of a number of steps. Exemplary steps include amplicon fragmentation, nucleic acid end repair, adapter ligation, barcoding, and amplification, such as amplification with indexed or barcoded primers. For example, amplicons in an amplicon sample can then be end-repaired. The end-repaired fragmented amplicons can then be ligated to adapters. The adapters can then be used to amplify the ligated amplicons. The primers used in the amplification process can be indexed or barcoded to generate indexed or barcoded libraries.

In some versions, separate workflows are used to determine the sequences of the amplicons in the amplicon samples generated from different primer sets. “Separate workflow” in this context means that none of the amplicons or any other contents of the amplicon samples generated from different primer sets are combined or mixed in the process of determining the sequences. In some versions, a separate sequencing library is generated from each amplicon sample generated from different primer sets. In some versions, different amplicon samples generated from the same primer set can be barcoded and combined into a single sequencing library. This can occur even in cases in which amplicon samples generated from different primer sets are processed in a separate workflow.

In some versions, sequencing libraries from each separate amplicon sample are sequentially generated. In some versions, the sequencing libraries are generated from the amplicon samples in the same order in which the amplicon samples were generated. In some versions, a sequencing library from each previously generated amplicon sample is generated before each subsequent amplicon sample is generated.

In some versions, each sequencing library is separately sequenced in a sequencing instrument. In some versions, each sequencing library is sequentially sequenced in a sequencing instrument. In some versions, each sequencing library is sequentially sequenced in a sequencing instrument in the order in which the sequencing libraries were generated. In some versions, each previously generated sequencing library generated from an amplicon sample with a given primer set is sequenced in a sequencing instrument before each subsequent amplicon sample with a different primer set is generated.

In some versions, all the separate sequencing libraries are generated within a single enclosable library-generating space. “Single enclosable library-generating space” as used herein refers to a space suitable for generating a sequencing library that is enclosable with a barrier impassable to humans.

In some versions, the template nucleic acid is the same in all the amplification reactions. In some versions, the template nucleic acid and all the amplification reactions are from a single, split sample. In some versions, the template nucleic acid in all the amplification reactions are from the same subject. In some versions, the template nucleic acid in at least one amplification reaction is different from the template nucleic acid in at least one of the other amplification reactions. In some versions, the template nucleic acid in at least one amplification reaction is from a different sample than the template nucleic acid in at least one of the other amplification reactions. In some versions, the template nucleic acid in at least one amplification reaction is from a different subject than the template nucleic acid in at least one of the other amplification reactions. In some versions, the template nucleic acid in each amplification reaction is different. In some versions, the template nucleic acid in each amplification reaction is from a different sample. In some versions, the template nucleic acid in each amplification reaction is from a different subject.

It is understood that any given amplification reaction of the invention may comprise primers other than those that are members of a set of primers of the invention.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### Tagged Primer Sets: An Approach for Overcoming Post-Amplification Contamination in Highly Sensitive Molecular Assays

### Introduction

Polymerase chain reaction (PCR) has been in use in molecular biology for decades, allowing scientists to take small amounts of DNA and generate numerous copies in an exponential manner using target-specific DNA primers (Garibyan et al. 2013). Repeated use of the same primers can lead to false positive results, as DNA from previous experiments can contaminate subsequent reactions. Sterilization between experiments and physical separation of lab spaces are used to reduce contamination risks (Mifflin 2007, Aslanzadeh 2004), but the increasing use of ultra-sensitive assays requires the development of techniques to identify, track, and exclude any contamination noise that may remain.

One ultra-sensitive assay we have recently developed is Structural Variant Enrichment and Normalization (SVEN) (WO 2025/006244 A1). This assay is designed to detect minimal residual disease in cancer patients after diagnosis using personalized structural variant (SV) target panels. Briefly, the process identifies dozens of SV breakpoints using tumor tissue from the patients. Then, PCR primer multiplexes are used to amplify these SVs from patient samples if they are present, followed by sequencing of the resulting DNA products. When tumor DNA fragments are present, even in trace amounts, we expect the SVEN to amplify these fragments so that they can be detected upon sequencing. However, we expect very few SV molecules to be present in patient blood samples. If there is carryover of residual PCR amplicons from one experiment to the next, this could lead to false positives. The current invention was designed to help identify such run-to-run contamination noise, and eventually improve our ability to detect true low-abundance tumor signal.

### Identification of Contamination Source

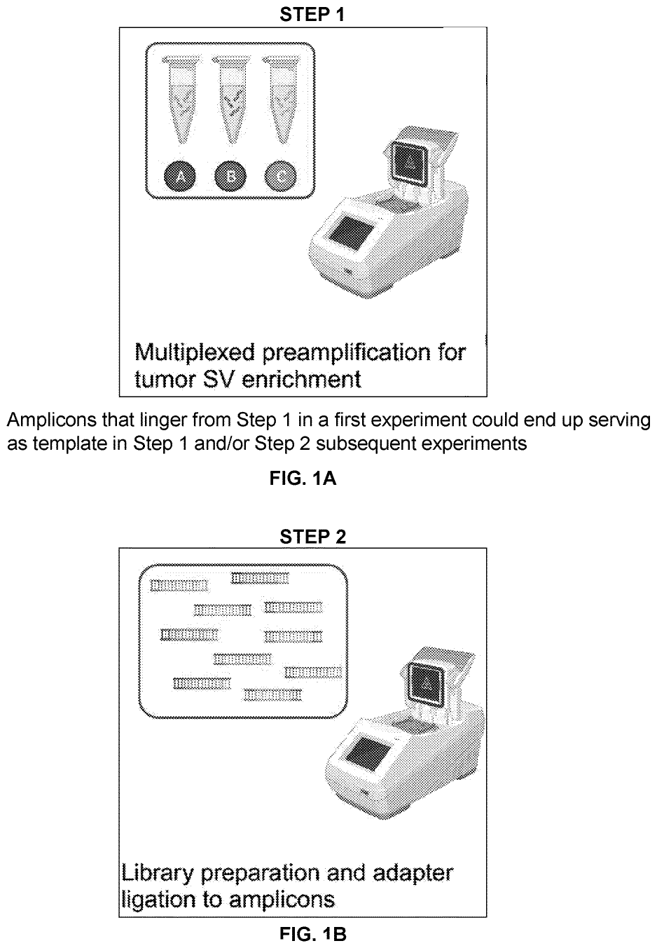

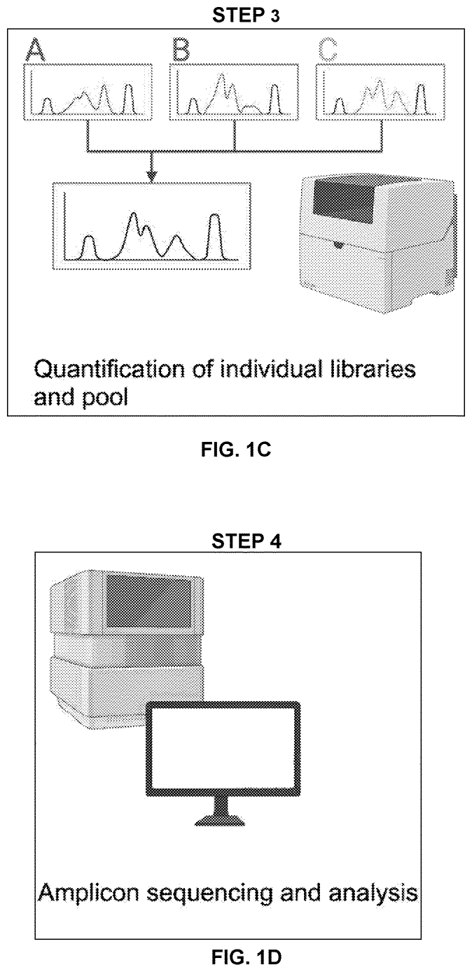

While conducting SVEN development experiments on cell line DNA, we found increasing amounts of contamination in our no-template controls (NTCs). The impact of contaminating DNA varies significantly depending on when it is introduced (FIG. 1): If the contamination occurs before exponential amplification, it will result in a very large number of contamination-derived amplicons in our sequencing data as these amplicons will participate in the exponential amplification reaction. If carry-over contamination occurs post-amplification at step 2 (library construction), however, few amplicons will be detected in our sequencing data. The number of amplicons in our NTCs was more consistent with post-amplification contamination during library preparation.

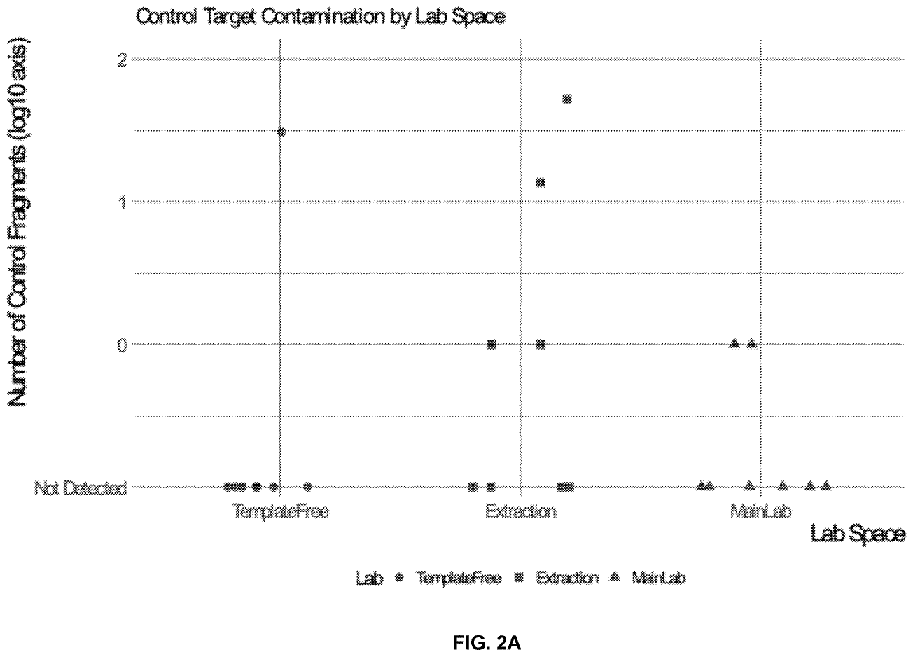

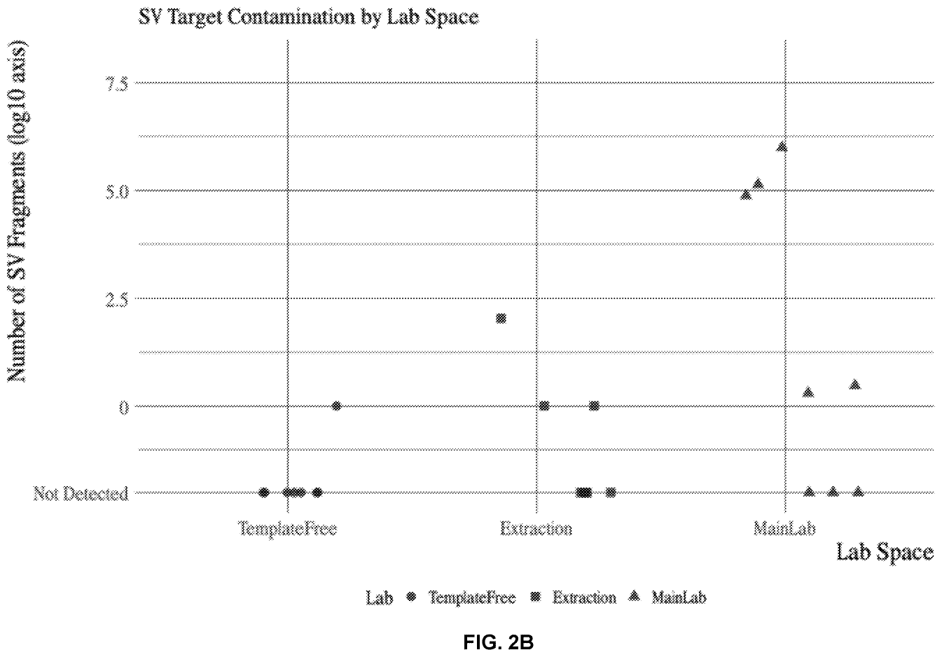

To further validate this finding, we prepared replicate NTCs in three physically separate lab spaces with increasing levels of potential contamination: a template-free lab (following standard procedure; circle), a DNA extraction lab (square), and the main lab space (triangle) using the HCC1954 multiplex with untagged forward primers (Table 1). Our results (FIGS. 2A and 2B and Table 2) confirmed that the number of contamination-derived amplicons was low in NTCs prepared in the template-free lab (no DNA extracted, prepared, or manipulated) and the extraction lab (where DNA is extracted, prepared, or manipulated), consistent with post-amplification contamination. In contrast, there was likely exponential amplification of contaminants in the NTCs prepared in the main lab space. The number of replicates with evidence of contamination was also higher in the main lab space and extraction lab (6/8 each) and lowest in the template-free lab (1/8). Importantly, the character of the contamination was different between the extraction and main labs despite having the same number of contaminated replicates. In the extraction lab, there was an even split between replicates that had control target contamination (4/8) and SV target contamination (3/8) including a replicate that had both control and SV contamination, which is consistent with the presence of HCC1954 tumor DNA in the extraction lab. Conversely, most of the replicates in the main lab had SV contamination (5/8) and only 2/8 replicates with control target contamination, including a replicate that had both control and SV contamination. Together, this indicates that the run-to-run carry-over contamination is significant, leading to indeterminate or even invalid results.

TABLE 1
Targets, tags, and primers employed in the experiments of FIGS. 2A and 2B.*

| | | Forward Primer | Reverse Primer |
|---|---|---|---|
| Target | Tag | Sequence | Sequence |
| HCC1954v2_DNAIA_short1 | — | CTTGTACCTGGTGTTC | GTAGGCTGGGTAGCCAAA |
| | | GGTTAT (SEQ ID NO: 1) | TC (SEQ ID NO: 2) |
| HCC1954v2_DNAIA_short2 | — | CCAGCCCAAGATGGA | CTCTTCTTTCCTTTCAGCA |
| | | TGAAT (SEQ ID NO: 3) | ACAC (SEQ ID NO: 4) |
| HCC1954v2_DNAIA_short3 | — | GCGGTGGAAGTCATT | TCTCTCTCTCTCCAGTGAA |
| | | CTGATA (SEQ ID NO: 5) | CAA (SEQ ID NO: 6) |
| HCC1954v2_DNAIA_short4 | — | GAGGGCCACAGATTG | GATCACTTAGCTCAGGAA |
| | | GTATT (SEQ ID NO: 7) | ACAAAC (SEQ ID NO: 8) |
| HCC1954v2_DNAIA_short5 | — | ACTGAAAGGTCAGAT | TACCGGTTTGTGTCCAAG |
| | | TCAGGAG (SEQ ID | AG (SEQ ID NO: 10) |
| | | NO: 9) | |
| HCC1954v2_CWSV00079 | — | TGAGGCTCGACAGGT | GCCTGTCGTGCAGCCATG |
| | | ACCATATAGCCT (SEQ | AGTTAG (SEQ ID NO: 12) |
| | | ID NO: 11) | |
| HCC1954v2_CWSV00085 | — | GACTCCAACCATTGG | AAAGGAGAACTATGTGAG |
| | | TATAATCTGAAAGTT | CTAATGCATTT (SEQ ID |
| | | GTCT (SEQ ID NO: 13) | NO: 14) |
| HCC1954v2_CWSV00096 | — | CTGCCTGACAACGGA | CTGTATATCTGGCTAGCC |
| | | CTGAAA (SEQ ID | ATATGCAGAAA (SEQ ID |
| | | NO: 15) | NO: 16) |
| HCC1954v2_CWSV00142 | — | TCGGCTATTTCTAACA | CAATCAGAAGACTGCTAG |
| | | GCTCACATGTTTACAT | GGTGGCTGTATT (SEQ ID |
| | | (SEQ ID NO: 17) | NO: 18) |
| HCC1954v2_CWSV00166 | — | TTCCATAAGTGCCAG | GAGTGTATAGACTAGAAG |
| | | CTGGCTGAGAAATA | TACAGCCATATAGTT (SEQ |
| | | (SEQ ID NO: 19) | ID NO: 20) |
| HCC1954v2_CWSV00203 | — | TCTGCTCCCAGGTTCA | CCCTGTTGCCCAGGCTAG |
| | | AGTGATTCTCAAG | AGTG (SEQ ID NO: 22) |
| | | (SEQ ID NO: 21) | |
| HCC1954v2_CWSV00281 | — | TCCGAGAGCTCACAG | GAGCGAGCTCAAGATAGA |
| | | CTTGGCT (SEQ ID | GGCAGCTAA (SEQ ID |
| | | NO: 23) | NO: 24) |
| HCC1954v2_CWSV00291 | — | GTTTGATGGAATTCA | TCCACATCACGGGAGCAG |
| | | CCAGTGTGTTT (SEQ | CTG (SEQ ID NO: 26) |
| | | ID NO: 25) | |
| HCC1954v2_CWSV00362 | — | ATTTAGAGAGCCTGT | GTCCAGCTGAGTGGAACA |
| | | CACAAATGTCCATAT | TTGAAAGAACA (SEQ ID |
| | | GAGAA (SEQ ID NO: 27) | NO: 28) |
| HCC1954v2_CWSV00739 | — | GAGAGGAGAGAGTGC | TAGGAGGAGCCCATTGGC |
| | | AGGGTCAA (SEQ ID | C (SEQ ID NO: 30) |
| | | NO: 29) | |
| HCC1954v2_CWSV00742 | — | ACATTATTGTTAACA | GCAGGAGAATGGCATGA |
| | | AGGGTTGCTTCTGGG | ACCCGG (SEQ ID NO: 32) |
| | | AAGA (SEQ ID NO: 31) | |
| HCC1954v2_CWSV00743 | — | CCCTGGCACCTAGTG | AGATGTGGCTGAACTTTA |
| | | GGTCCATAC (SEQ ID | AATGCATATT (SEQ ID |
| | | NO: 33) | NO: 34) |
| HCC1954v2_CWSV00744 | — | GCACTCCAGAGACTC | AAAGGGCCAGGCCTGAGT |
| | | TGTTTCTAAA (SEQ ID | GGT (SEQ ID NO: 36) |
| | | NO: 35) | |
| HCC1954v2_CWSV00791 | — | CAGGCATGGTAGCGC | CTCCGGCACTGGGCAGAG |
| | | GCCT (SEQ ID NO: 37) | GTA (SEQ ID NO: 38) |
| HCC1954v2_CWSV00841 | — | GGAAGCCTCATAATC | TCAGTGCTAGCTGATAAC |
| | | ATGGTGGAAGGCA | TCTGACTTT (SEQ ID |
| | | (SEQ ID NO: 39) | NO: 40) |
| HCC1954v2_CWSV00852 | — | CATGTTTACCGATGA | CACTGCATGTTCCCAGTC |
| | | AGCTGCAAA (SEQ ID | ATAGGTGG (SEQ ID |
| | | NO: 41) | NO: 42) |
| HCC1954v2_CWSV00856 | — | AGGAAGCTCAGCTAT | AGACCCATCTCACATGTA |
| | | ACCTGTTACTGATAAT | GACACA (SEQ ID NO: 44) |
| | | TT (SEQ ID NO: 43) | |
| HCC1954v2_CWSV01009 | — | CCCATGCAGGCCTAG | CTTCTCCAAGTCTCTACAC |
| | | GCTAGTGT (SEQ ID | TTGGCCACAA (SEQ ID |
| | | NO: 45) | NO: 46) |
| HCC1954v2_CWSV01021 | — | ACCTAAAGGACACAT | AGGATGACGCCTGGCTTG |
| | | CTCAGTACATTT (SEQ | GAGAAT (SEQ ID NO: 48) |
| | | ID NO: 47) | |
| HCC1954v2_CWSV01028 | — | ACTGAAGGCTCTATG | AGCAACAAAGATTATCCT |
| | | TGCAGGAACTCC (SEQ | AAGACTTCTGGAGGAAG |
| | | ID NO: 49) | (SEQ ID NO: 50) |
| HCC1954v2_CWSV01070 | — | CCTCCCAAAGTGCTG | AGGAGGGAGTGACAGGA |
| | | GGATTATAGGCA | TGTGATCTG (SEQ ID |
| | | (SEQ ID NO: 51) | NO: 52) |
| HCC1954v2_CWSV01075 | — | AACTGTTCTTCCAGGC | AGCCTGGCCAACACAGTG |
| | | CTGATTT (SEQ ID | AAACC (SEQ ID NO: 54) |
| | | NO: 53) | |
| HCC1954v2_CWSV01077 | — | CCCATCCTGGCACGT | TGACCTGTTCTTGGGTAG |
| | | ACTGCTACT (SEQ ID | CACTATTGGT (SEQ ID |
| | | NO: 55) | NO: 56) |
| HCC1954v2_CWSV01100 | — | AGGCACACTCAGGCT | AAAGGGATAGCAGTTTCC |
| | | TTACGATTCTTGTTTA | TGCTGTCCATAG (SEQ ID |
| | | (SEQ ID NO: 57) | NO: 58) |
| HCC1954v2_CWSV01109 | — | ATTCTCCCTGCAAATT | CCCAGGCTGCTGGTCATG |
| | | GTTTGACTCTAGAAA | GAAA (SEQ ID NO: 60) |
| | | TAGC (SEQ ID NO: 59) | |
| HCC1954v2_CWSV01130 | — | GGGTAGTAGCGATCT | CACAGGGTTTATCTCCTC |
| | | GGGTTACCCAG (SEQ | AGACAAA (SEQ ID NO: 62) |
| | | ID NO: 61) | |
| HCC1954v2_CWSV01177 | — | CATTGACCCACCGGT | GCGGACGCCATGGAGCT |
| | | CATTCAGGAG (SEQ ID | (SEQ ID NO: 64) |
| | | NO: 63) | |
| HCC1954v2_CWSV01529 | — | CTGAGGGTTATCCAA | AACACATTAGTGATGTCT |
| | | GTGACCCATTGCA | CAATGCCATCC (SEQ ID |
| | | (SEQ ID NO: 65) | NO: 66) |

*SV targets and primers are indicated with “CWSV.” Control targets and primers are indicated with “DNAIA.”

TABLE 2
Contaminated replicates by lab space.*

| Target-type | Template-Free | DNA Extraction | Main Lab |
|---|---|---|---|
| Contamination | Lab | Lab | Space |
| Control only | 0 | 3 | 1 |
| SV only | 0 | 2 | 4 |
| Both | 1 | 1 | 1 |
| Total | 1 | 6 | 6 |

*The Control only rows enumerates replicates that only had control contamination. The SV-only row enumerates replicates that had only SV target contamination. The Both row enumerates replicates that had contamination of both control and SV targets. Total is the total number of contaminated replicates with any combination between the target types.

### Tagged Primer Design and Use

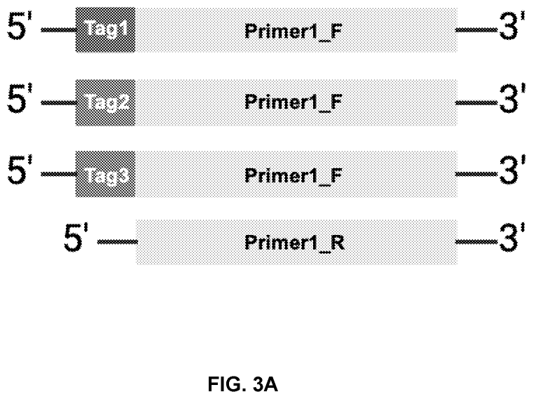

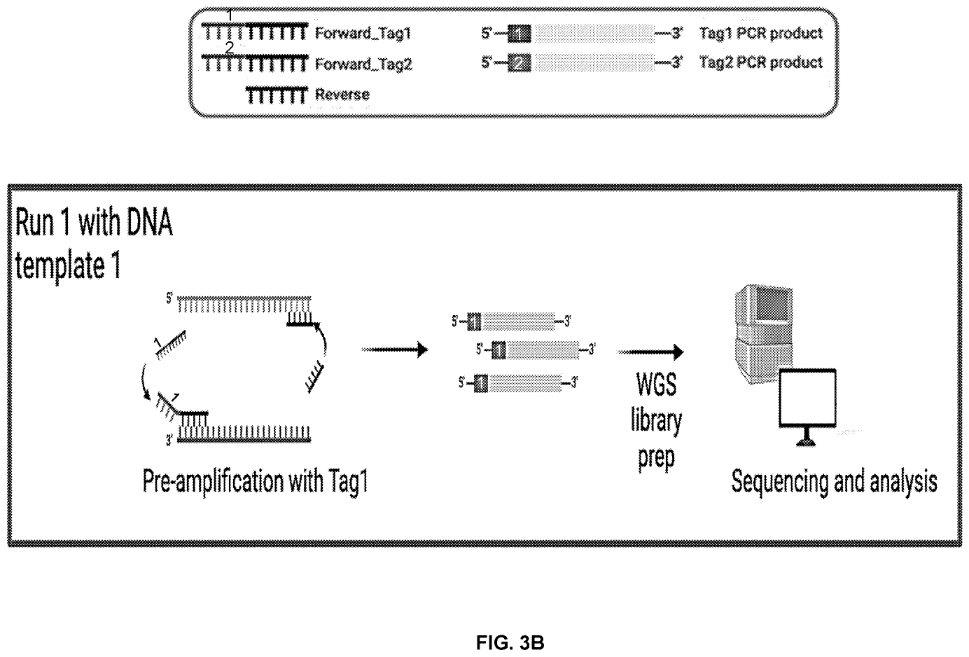

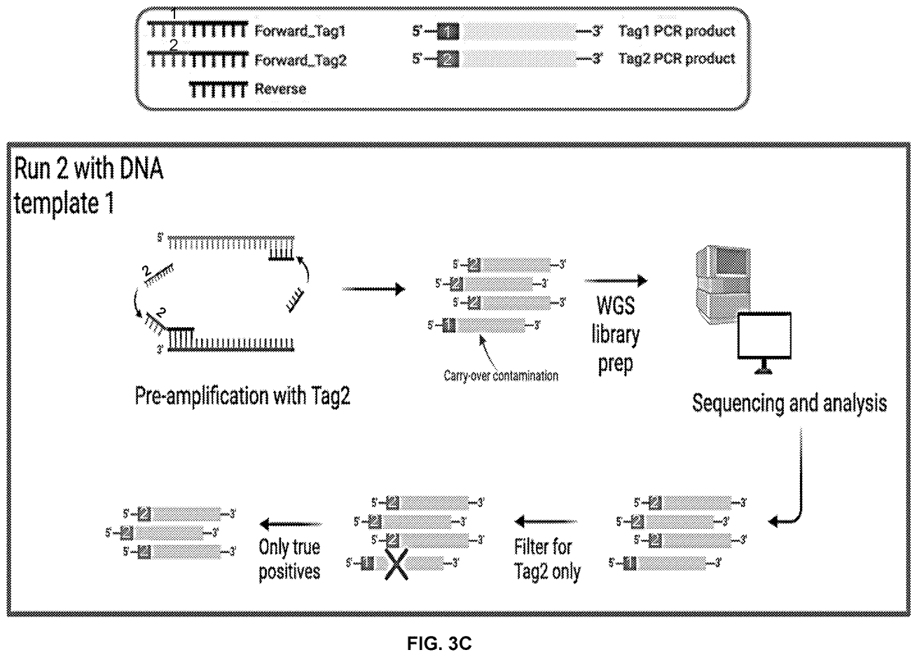

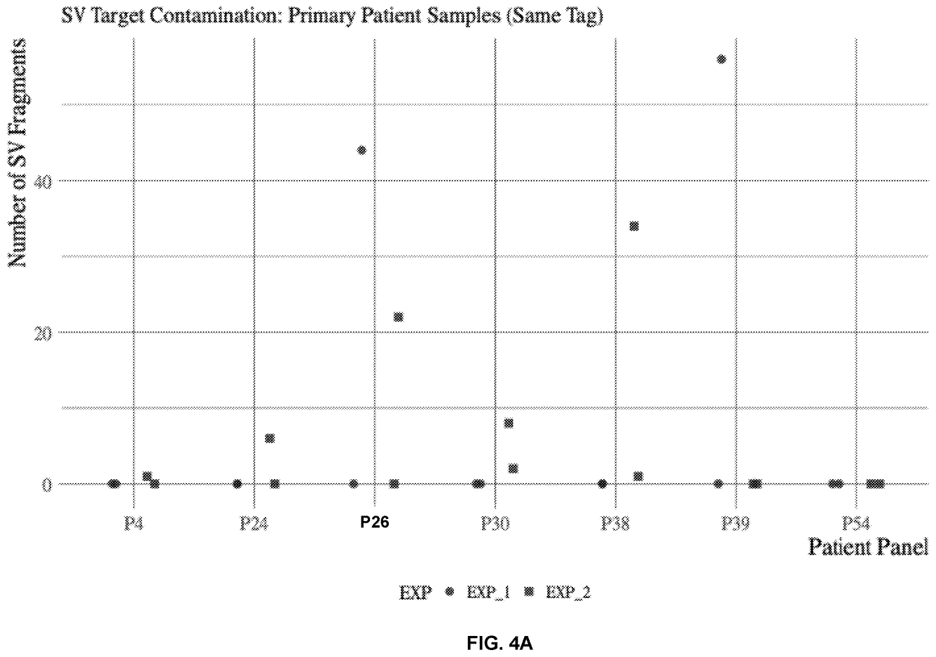

As all sequencing library preparation after initial amplification must occur in the main lab (post-PCR space), we expected that it would be impossible to completely eliminate DNA from previous experiments. We therefore had to develop an approach to identify and remove post-amplification contamination. We reasoned that developing multiple versions of each primer panel, differing only in the addition of a few base pairs to the 5′ end of the forward primers (FIGS. 3A-3C), would allow us to identify reads that came from different experiments without significantly impacting primer performance. For each of the three versions of the primer sets, the target-specific sequence in the forward primer remains constant, while a four base pair tag is added to the 5′ end of the forward primer. All amplicons generated in a given experiment would, therefore, contain the same tag. We could then rotate through primer sets with different tags in subsequent experiments.

This approach provides a means to remove the contaminating sequences from each sample at the computational analysis stage by requiring the correct tag sequence be present on a fragment before it is included in the analysis. The source experiment of contaminating DNA from previous experiments can also be determined using their tag, enabling quantification and source tracking of between-run contamination wherein high levels of contaminating, improperly tagged reads would indicate a pre-amplification contamination event (step 1 of FIG. 1) while low levels of would indicate a post-amplification event (step 2 of FIG. 2).

The tags were limited to four base pairs in the present examples to minimize potential impacts of the tag on primer secondary structure and thermodynamics. Furthermore, the reverse primer was left untagged to maintain fluorophore diversity on the Illumina sequencer in the first cycles to prevent errors in the sequencing run. However, additional tag sets could be developed for the reverse primers to increase the possible combinations, which would be particularly powerful in environments with high-throughput, repeated sampling. The tagged system was tested using one of the multiplexes developed for our cell lines (HCC1954) to ensure that the performance of the multiplex was not significantly affected by the addition of the tags, and the results demonstrated that the primers were still working as expected. Although there was some impact on absolute quantification between panels, relative quantification between samples using the same tagged panels remained stable between experiments. We proceeded to use them in subsequent panel designs and testing for both our cell line model system and primary patient samples.

### Longitudinal Use and Results

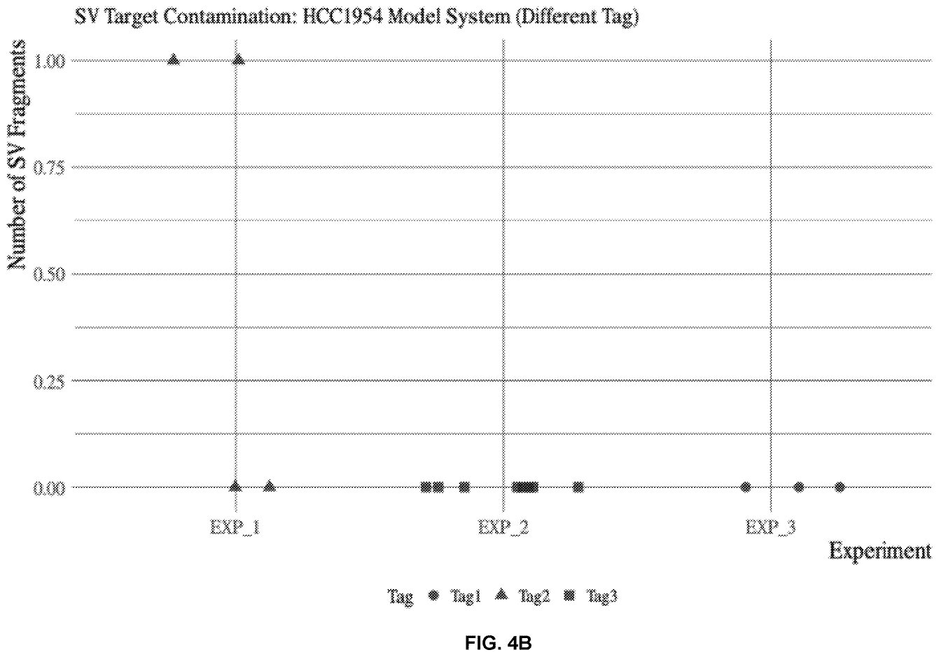

In subsequent testing using patient-specific SV panels, we found that when the same primer tags (Tag3) are used between consecutive experiments (Table 3), residual amplicons persist (FIG. 4A), and higher levels are often found the second time a panel was used in several, but not all, the panels tested. However, implementation of rotating tagged primer sets (Table 4), which is the intended use-case for this technology, allowed us to filter out carry-over contamination across runs using the 0% tumor fraction HCC1954 model system. We would expect no SV contamination in this model system since now tumor content is present. Our results demonstrate no carry-over contamination in the transition from EXP_1 (n=4; Tag2, circle), with two SV targets identified, to EXP_2 (n=8; Tag3, square) and EXP_3 (n=3; Tag1, triangle), which both had no SV targets identified (FIG. 4B). These results demonstrate a simple and effective method to identify, quantify, and exclude post-amplification product contamination in DNA sequencing experiments. Such a system has utility in applications where low-abundance target DNA needs to be accurately identified and quantified.

TABLE 3
Targets, tag, and, primer sequences for two consecutive experiments using the same tag
(underlined) for templates P4, P24, P26, P30, P38, P39, P54 as shown in FIG. 4A.*

| | | | |
|---|---|---|---|
| Target | Tag | Forward Primer Sequence | Reverse Primer Sequence |

P4

| | | | |
|---|---|---|---|
| p04_CWSV00001 | 3 | CATGTTCATCACACACATG | CAGAATCCATGCTCTTAACC |
| | | CTAACACAAATCCAGTC | ATTATGCTCTGC (SEQ ID |
| | | (SEQ ID NO: 448) | NO: 449) |
| p04_CWSV00007 | 3 | CATGGACCGCCGGTGAGC | GAATGTACTTTGCAAGTGAG |
| | | CGG (SEQ ID NO: 450) | AATGACAT (SEQ ID NO: 451) |
| p04_CWSV00013 | 3 | CATGTCATCCTGGACATTA | ACCCAGAGTAGGCATTAATC |
| | | GCTACCTCTGTGATTGA | ATTCCAGACATTTC (SEQ ID |
| | | (SEQ ID NO: 452) | NO: 453) |
| p04_CWSV00017 | 3 | CATGGCACTTGCCTCCACC | GGTGAAACTACTTAATGGGT |
| | | TTCCCAG (SEQ ID NO: 454) | GCAGGGTG (SEQ ID NO: 455) |
| p04_CWSV00028 | 3 | CATGCCAGGGACCCTAGC | TGACCCTGGTCAGTATTCCT |
| | | AAACGAGCT (SEQ ID | AGGGTCC (SEQ ID NO: 457) |
| | | NO: 456) | |
| p04_CWSV00031 | 3 | CATGCCATATTTATACCAG | TCATAGCAAGCAATGACAA |
| | | CCTGGCTCTAGGAGAGT | TGCCAATAAA (SEQ ID |
| | | (SEQ ID NO: 458) | NO: 459) |
| p04_CWSV00038 | 3 | CATGTCTCCCACACTCCAG | TGTAGATACAGTTATTCTTT |
| | | ATCCAAAGACTACTT (SEQ | GTTACTGGTTGAAT (SEQ ID |
| | | ID NO: 460) | NO: 461) |
| p04_CWSV00040 | 3 | CATGCGTGCACGCGCATG | CAAATAAGCACATAAGATA |
| | | CAAATAGG (SEQ ID | AAGATCCAGCTATTT (SEQ |
| | | NO: 462) | ID NO: 463) |
| p04_CWSV00042 | 3 | CATGCCCTGAACATTTCAC | GCCTAAGCCTGGAACCTAG |
| | | CATGAAATTTGTGAGCT | GAGGTG (SEQ ID NO: 465) |
| | | (SEQ ID NO: 464) | |
| p04_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p04_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p04_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p04_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p04_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

P24

| | | | |
|---|---|---|---|
| p24_CWSV00001 | 3 | CATGGTTGAACCTGGGAG | GCTCAGGCACTGAGAGTCA |
| | | GCGGAGGT (SEQ ID NO: 67) | GCC (SEQ ID NO: 68) |
| p24_CWSV00005 | 3 | CATGCAGGAGGCCAAGGG | GGATAGCTATTGAGAGCTTC |
| | | TGACGG (SEQ ID NO: 69) | CTTCATTTG (SEQ ID NO: 70) |
| p24_CWSV00008 | 3 | CATGAAGAGTACTTGACA | ATGAAGTATCATATAAGGAT |
| | | CATGCATGAACTCAGTAA | ACACTCTATGCATC (SEQ ID |
| | | ATT (SEQ ID NO: 71) | NO: 72) |
| p24_CWSV00011 | 3 | CATGGGGTCCCACATGAC | GGTCCCAGTCCCTCAGTCTC |
| | | AGCCATGACT (SEQ ID | TCCA (SEQ ID NO: 74) |
| | | NO: 73) | |
| p24_CWSV00012 | 3 | CATGAAAGAAACAAACTT | ACAGTTCAGTAGTGTTAAGA |
| | | GTCTCGGTATCCAGAAAGT | ACATTTGCA (SEQ ID NO: 76) |
| | | GT (SEQ ID NO: 75) | |
| p24_CWSV00016 | 3 | CATGAACTATTCCTTCAAG | GGATGGCACCTGCTGGCTAC |
| | | CCAAGCATAGTATGTCTAG | ATTT (SEQ ID NO: 78) |
| | | A (SEQ ID NO: 77) | |
| p24_CWSV00022 | 3 | CATGAGTAGTTGGGATTAC | AAGACACAAGTTTATCTATA |
| | | AGGCATGTGCCA (SEQ ID | TGACAAACTTTGAC (SEQ ID |
| | | NO: 79) | NO: 80) |
| p24_CWSV00023 | 3 | CATGTGTAACTAACCTGCA | TGCCCATTGTATTAGTCAGG |
| | | CGTTGTGCACATG (SEQ ID | CAGGGTT (SEQ ID NO: 82) |
| | | NO: 81) | |
| p24_CWSV00028 | 3 | CATGCTCCTGGACTCAAGC | GGAGCTGGTGGAGCCTGCTC |
| | | GATCCTCCTG (SEQ ID | T (SEQ ID NO: 84) |
| | | NO: 83) | |
| p24_CWSV00040 | 3 | CATGAATATGAAATAAAG | GTGACCAAACTAAATGTTTG |
| | | CCCACAGTAAAGATTACTC | TGTACTTTAAAGGA (SEQ ID |
| | | T (SEQ ID NO: 85) | NO: 86) |
| p24_CWSV00044 | 3 | CATGGCCACTGCACTCCAG | GTGGCCCTAACTCGGCTCAC |
| | | CCTAGGT (SEQ ID NO: 87) | CATA (SEQ ID NO: 88) |
| p24_CWSV00046 | 3 | CATGAAAGAATATCGATA | AAATAGCTGCTTTAGATATC |
| | | CATGGGCAGCTGACACTT | TGAAGCCA (SEQ ID NO: 90) |
| | | (SEQ ID NO: 89) | |
| p24_CWSV00049 | 3 | CATGTTTGAGACACAGTTT | CCCACTGTGGAATGTCACAG |
| | | CTCTCTTGTCACCCAG | CTATTT (SEQ ID NO: 92) |
| | | (SEQ ID NO: 91) | |
| p24_CWSV00050 | 3 | CATGCCCACTGCTGGACCT | TGATGCATGGCCTTAGCTGT |
| | | TAGCACG (SEQ ID NO: 93) | TTACTTTGC (SEQ ID NO: 94) |
| p24_CWSV00053 | 3 | CATGCTCCACATGCATAGT | GAGGTGGAAATTGCAGTGA |
| | | ATCAGCCCTCCA (SEQ ID | GCCGAG (SEQ ID NO: 96) |
| | | NO: 95) | |
| p24_CWSV00058 | 3 | CATGAGGTGTGAGCCACC | AGTGACTCACGCCTATAATC |
| | | ACGCCT (SEQ ID NO: 97) | CCAGCG (SEQ ID NO: 98) |
| p24_CWSV00061 | 3 | CATGTTTAAAGACAGTCTT | CCGAGGCAGGCCGATCACC |
| | | CGCCAGGTGTGGT (SEQ ID | (SEQ ID NO: 100) |
| | | NO: 99) | |
| p24_CWSV00068 | 3 | CATGTTTACGAGGCCAGC | GTAACATAATACAAAGCTG |
| | | ATCATCCTGATACTAAA | CATCACAGCTAATCT (SEQ |
| | | (SEQ ID NO: 101) | ID NO: 102) |
| p24_CWSV00078 | 3 | CATGGGGATCCCAGACCTT | AGGTGCTTATAAATTTCTGT |
| | | GGCCAGG (SEQ ID NO: 103) | TACATGAAAGAATAC (SEQ |
| | | | ID NO: 104) |
| p24_CWSV00087 | 3 | CATGAAATTAAGAAGGTT | ATTCCCGCTCTTATGGCTTA |
| | | CATCACAGTTCAAAGAAG | TACTCTAGTTGG (SEQ ID |
| | | CTC (SEQ ID NO: 105) | NO: 106) |
| p24_CWSV00088 | 3 | CATGTGGTCTGGTCGTCCA | CACAGTTCTCATACTCAGGA |
| | | TGCTTGCA (SEQ ID NO: 107) | ACCCTCAT (SEQ ID NO: 108) |
| p24_CWSV00090 | 3 | CATGTGGTGTTGGCGCTCA | TTTGTCAGTTGGCCCTGATT |
| | | GACAGTGATA (SEQ ID | TCAGATATCCTC (SEQ ID |
| | | NO: 109) | NO: 110) |
| p24_CWSV00097 | 3 | CATGGCTTGTAATCCCAGC | TTTCACCAGCACAGTGTAGA |
| | | TACTCAGGAGGC (SEQ ID | ATTGCAG (SEQ ID NO: 112) |
| | | NO: 111) | |
| p24_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p24_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p24_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p24_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p24_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

P26

| | | | |
|---|---|---|---|
| p26_CWSV00006 | 3 | CATGACTACATTTCCTGCT | TCCGAAGGAAGTGAATTCA |
| | | ATCTTGATTTACAACTCTT | CCCAAGGAT (SEQ ID |
| | | G (SEQ ID NO: 118) | NO: 119) |
| p26_CWSV00010 | 3 | CATGTTTGGGATGAAACTG | TTACTGCCAGAGTATTCTGG |
| | | TTCCACCTCAGATTT (SEQ | TTAAGAAAC (SEQ ID |
| | | ID NO: 120) | NO: 121) |
| p26_CWSV00012 | 3 | CATGCATAGTATTTCACAT | AAACGTTTGGTTCATTACAA |
| | | TTGTGTATTACTTCGAATT | GAATAGAGGCCTTC (SEQ ID |
| | | T (SEQ ID NO: 122) | NO: 123) |
| p26_CWSV00014 | 3 | CATGTCCTTGAGTCTATCT | TGCGTGGTGTGCAGTAAATT |
| | | TGGAGGTGAGCAAAC (SEQ | ATCAATAGG (SEQ ID |
| | | ID NO: 124) | NO: 125) |
| p26_CWSV00017 | 3 | CATGCCAAATTTAATTCAG | ACTCTTTATGAAGAGCAATT |
| | | TCTTTCTTCCCATAGTGTC | TGGTAGTACCTACCA (SEQ |
| | | T (SEQ ID NO: 126) | ID NO: 127) |
| p26_CWSV00022 | 3 | CATGGTTCAACACCCTTTG | GGCCTCCTTGAACTATGGTG |
| | | ATGGCTCCCATG (SEQ ID | GGCT (SEQ ID NO: 129) |
| | | NO: 128) | |
| p26_CWSV00030 | 3 | CATGAAAGCAGTGTGCTG | TCCTCATGTGAGTTTATGTC |
| | | AGTGAAAGAAGTCAG | CTACTTGAA (SEQ ID NO: 131) |
| | | (SEQ ID NO: 130) | |
| p26_CWSV00038 | 3 | CATGTTGTGCTGCAATAAA | AGTCCAAAGTTTAAGATGCC |
| | | CATAGATGCGCG (SEQ ID | AGAGATGCA (SEQ ID |
| | | NO: 132) | NO: 133) |
| p26_CWSV00039 | 3 | CATGTTTCTCCATAGACTC | TTGATATAAAGCCTACTGTG |
| | | CTGTTCTTGTTATACAGAT | ATCTTGGCACCATAC (SEQ |
| | | G (SEQ ID NO: 134) | ID NO: 135) |
| p26_CWSV00041 | 3 | CATGTTGTGGATGATTAGC | TCAGGAGTTTGAGATCAGCC |
| | | ATCTTAACCATGTTGAGTC | TGGCC (SEQ ID NO: 137) |
| | | T (SEQ ID NO: 136) | |
| p26_CWSV00042 | 3 | CATGCCCAGATGTGGATGT | GGGTGGATGAGACTTTCCGA |
| | | TCATGGTCCAGA (SEQ ID | AGAAC (SEQ ID NO: 139) |
| | | NO: 138) | |
| p26_CWSV00046 | 3 | CATGCCCTGCGAACCGTCG | GTGGATTACCTGAGGTCAGT |
| | | ACCG (SEQ ID NO: 140) | AGTTCAAGA (SEQ ID |
| | | | NO: 141) |
| p26_CWSV00052 | 3 | CATGGCAGGCTGCTGGAC | CCTGGCTAGCTCCGAAATAT |
| | | CCTGC (SEQ ID NO: 142) | TCCAGGA (SEQ ID NO: 143) |
| p26_CWSV00053 | 3 | CATGGGTTCCTTCGCTACT | CCACCACACCCAGCTAAGTT |
| | | CTGGCTCCA (SEQ ID | T (SEQ ID NO: 145) |
| | | NO: 144) | |
| p26_CWSV00055 | 3 | CATGGAATCTCAAACGGT | CTGCATAGAATACAGCCTTA |
| | | ATTTAACATATAAGAAGA | CTGAATGTTAGTCTC (SEQ |
| | | TTC (SEQ ID NO: 146) | ID NO: 147) |
| p26_CWSV00057 | 3 | CATGAAGGCTCCTGGAGC | TCTGTCTGCCTGGAGCACTG |
| | | CCTTTGAGG (SEQ ID | ATTTCTC (SEQ ID NO: 149) |
| | | NO: 148) | |
| p26_CWSV00058 | 3 | CATGTATTGGATAGTGAGC | CCTGAGCTAGATATAAAGA |
| | | CCACCACTCAGTAACA | CTCTCCACGT (SEQ ID |
| | | (SEQ ID NO: 150) | NO: 151) |
| p26_CWSV00062 | 3 | CATGACCACCACGCCTGG | TTTAAGCCAATAGCACCATG |
| | | CTAATTT (SEQ ID NO: 152) | TTTCTTT (SEQ ID NO: 153) |
| p26_CWSV00064 | 3 | CATGGGGCATTGCAGCAG | GGCATTTATTGCAGGGTGCC |
| | | GTGCAG (SEQ ID NO: 154) | AAGC (SEQ ID NO: 155) |
| p26_CWSV00066 | 3 | CATGCCTTAAACCATCACG | AGCCTATGTGTGTCTCTGCA |
| | | GTGCTGCTTTGA (SEQ ID | CGTGAT (SEQ ID NO: 157) |
| | | NO: 156) | |
| p26_CWSV00071 | 3 | CATGATCTTACAAGAAGA | CTGCTGAGAAATTACTGTCC |
| | | CATATGCCCACAGAGGAC | CTGTGCAAA (SEQ ID |
| | | TA (SEQ ID NO: 158) | NO: 159) |
| p26_CWSV00072 | 3 | CATGCTGGGCATCAGAGC | CCCGTGCCACACTCGCCG |
| | | AAGACTCCATCT (SEQ ID | (SEQ ID NO: 161) |
| | | NO: 160) | |
| p26_CWSV00074 | 3 | CATGCTGGGCATGCGACA | ATCGTCAAATGAACTCGAA |
| | | TCTCTCAGG (SEQ ID | ATGAATCATAATCGA (SEQ |
| | | NO: 162) | ID NO: 163) |
| p26_CWSV00081 | 3 | CATGTTGCCCTGGTGCCTA | GCACAACCAGAGATACATC |
| | | GGCTGG (SEQ ID NO: 164) | AGCCCTAAA (SEQ ID |
| | | | NO: 165) |
| p26_CWSV00083 | 3 | CATGGCCCACCTTGGCCTC | GAATGCTTGTGTTTAAATCT |
| | | CCAAAG (SEQ ID NO: 166) | TCAGTGTCACTCTAG (SEQ |
| | | | ID NO: 167) |
| p26_CWSV00087 | 3 | CATGGGGAATAGACATGC | AGCTAATATCATGCCACTGC |
| | | TGTGGTGGCAGA (SEQ ID | ACTCCAGC (SEQ ID NO: 169) |
| | | NO: 168) | |
| p26_CWSV00092 | 3 | CATGATTAAGGATGAGGA | AGGCTTCACCAGACACCAA |
| | | CCCTGAGGTTCAGAAAC | ATCTGC (SEQ ID NO: 171) |
| | | (SEQ ID NO: 170) | |
| p26_CWSV00106 | 3 | CATGAGATCTGCTGTTAGT | CCTATGCGGCCGAGCCTCC |
| | | CTGATGGGCTTCC (SEQ ID | (SEQ ID NO: 173) |
| | | NO: 172) | |
| p26_CWSV00108 | 3 | CATGTTTCAGCCAAGCAGT | GGTCTCAAACTCCTGGGTTC |
| | | TTGAAAGGATTCT (SEQ ID | AAGTGAAC (SEQ ID NO: 175) |
| | | NO: 174) | |
| p26_CWSV00110 | 3 | CATGATTATGATAGGTATC | ACGAATTTGTGGCCAAGTCC |
| | | AACACCCTTGCATTT (SEQ | TCAAA (SEQ ID NO: 177) |
| | | ID NO: 176) | |
| p26_CWSV00115 | 3 | CATGGGAATCTTTGTTAAT | GGCTGTGGAGATGCATCAGT |
| | | TGTTTCACGTGGAAA (SEQ | GAATCAG (SEQ ID NO: 179) |
| | | ID NO: 178) | |
| p26_CWSV00118 | 3 | CATGTCCATTGCCATTACT | TTCTCATGACAGTGCTGATC |
| | | ATTTCAGAGTTACAATGTT | TGTGGATTT (SEQ ID NO: 181) |
| | | (SEQ ID NO: 180) | |
| p26_CWSV00120 | 3 | CATGCGAGCGGCCCAACT | TCTCAGCTACTCGGGAGGCT |
| | | CCACAG (SEQ ID NO: 182) | GAGAT (SEQ ID NO: 183) |
| p26_CWSV00134 | 3 | CATGAAACGAGTTAATAC | TCATGGCATGGATGTACCAA |
| | | TTGCACCGTCTCAGACA | AGATTT (SEQ ID NO: 185) |
| | | (SEQ ID NO: 184) | |
| p26_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p26_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p26_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p26_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p26_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

P30

| | | | |
|---|---|---|---|
| p30_CWSV00004 | 3 | CATGAAACACAACCACTG | TCCTGAGCCAGGTTTACATG |
| | | AGAGTGCTATTATGGTACT | GTTACCATT (SEQ ID NO: 187) |
| | | C (SEQ ID NO: 186) | |
| p30_CWSV00005 | 3 | CATGGGACATTCATGTTTC | GTCACCATGGCCACTCTGGA |
| | | TCCAAAGCCCTCTG (SEQ | GC (SEQ ID NO: 189) |
| | | ID NO: 188) | |
| p30_CWSV00016 | 3 | CATGGAAGTTGCCAAACA | ATCTATCCCAAAGGGCTGCT |
| | | GTTCCTCAGAATAGTTACA | GTGAGG (SEQ ID NO: 191) |
| | | C (SEQ ID NO: 190) | |
| p30_CWSV00030 | 3 | CATGTTGAATGAATGGTAC | GTCTGGGAGGCAGAGGTTG |
| | | GTGCGAAGTATATGCTGA | CAGT (SEQ ID NO: 193) |
| | | (SEQ ID NO: 192) | |
| p30_CWSV00031 | 3 | CATGCCACAAACCTCTTTG | CTGCCTGCTGGCAGGGCTTC |
| | | TTATAATTTCTCCAGTTGA | (SEQ ID NO: 195) |
| | | G (SEQ ID NO: 194) | |
| p30_CWSV00034 | 3 | CATGGGGATAGACAGACC | CAGATTCGCTAAATGATTGA |
| | | TGTTGAATCATAAATAGA | CCTTGTACATTT (SEQ ID |
| | | ACA (SEQ ID NO: 196) | NO: 197) |
| p30_CWSV00038 | 3 | CATGACCTTCACAAAGCAT | TGAGCTGTGGCCACTTACTC |
| | | GCACTGACTGT (SEQ ID | AGGAAA (SEQ ID NO: 199) |
| | | NO: 198) | |
| p30_CWSV00039 | 3 | CATGATCCCAGCACTCTGG | TGGTCCCTCTGGCCAACTTG |
| | | AGGGCC (SEQ ID NO: 200) | GC (SEQ ID NO: 201) |
| p30_CWSV00040 | 3 | CATGTTTGCCTACCTTCCT | CCAACAGATGCCTCCCACTC |
| | | GTACACATAAACCAAGG | CAAGT (SEQ ID NO: 203) |
| | | (SEQ ID NO: 202) | |
| p30_CWSV00041 | 3 | CATGTTACACATTATTCAG | CACCCACCTTGGCCTCCCAA |
| | | CAATGGACTAGAATTGGT | AG (SEQ ID NO: 205) |
| | | GC (SEQ ID NO: 204) | |
| p30_CWSV00053 | 3 | CATGCATGGCAGCATGGC | CTCCTGCCTCAGCCTCCCGA |
| | | AGCTTTATATCCTG (SEQ | G (SEQ ID NO: 207) |
| | | ID NO: 206) | |
| p30_CWSV00055 | 3 | CATGGGGCTTGGCCTTAAG | GACAGAGTTTCACTCTTGTT |
| | | TGCCGT (SEQ ID NO: 208) | GCCCAGG (SEQ ID NO: 209) |
| p30_CWSV00056 | 3 | CATGGGGTCCAGGAAAGT | GAGTTCAAGACCAGCCTCGC |
| | | CGGGCAG (SEQ ID NO: 210) | CAATG (SEQ ID NO: 211) |
| p30_CWSV00058 | 3 | CATGTTCACAGCTAATCTC | GATGGCTTGGAAATCCATAA |
| | | TCCTGGGCACAC (SEQ ID | GTCCTATTT (SEQ ID NO: 213) |
| | | NO: 212) | |
| p30_CWSV00083 | 3 | CATGTTTAACCTGCTCAGT | TGATCCCACCTGGTCTGATG |
| | | CTAGTTAGAACATCTCTCG | GGTG (SEQ ID NO: 215) |
| | | T (SEQ ID NO: 214) | |
| p30_CWSV00106 | 3 | CATGCACAGAGCATGAGC | GGTGCTCAGTATTCTAATTC |
| | | ATTGAAGCAGAATGAA | TTTGATTCCTTGTC (SEQ ID |
| | | (SEQ ID NO: 216) | NO: 217) |
| p30_CWSV00109 | 3 | CATGCACACACACCAGCA | CCAGCATGATGTTGGTCCAA |
| | | TGCACACTCA (SEQ ID | GTTGGC (SEQ ID NO: 219) |
| | | NO: 218) | |
| p30_CWSV00116 | 3 | CATGGGGCCAAAGCGGAC | GCCGTTGTTCTTATCATCTG |
| | | TGAGGC (SEQ ID NO: 220) | TCGATACCAAA (SEQ ID |
| | | | NO: 221) |
| p30_CWSV00119 | 3 | CATGAAAGACCCAGAATA | TCCGTGTGCCCAAGTCAGTT |
| | | GCCAAAGCTATCCTGAG | ACCA (SEQ ID NO: 223) |
| | | (SEQ ID NO: 222) | |
| p30_CWSV00126 | 3 | CATGTGGTCCTTTCTTGCA | TAATTGGTCAGTATCCGTAT |
| | | GTAGGAGGATGTC (SEQ ID | GGGTCTTT (SEQ ID NO: 225) |
| | | NO: 224) | |
| p30_CWSV00128 | 3 | CATGAAACTGCCTGATTCC | GAGCCTTCTACCTTCAGACT |
| | | ACAGCTGAGGTA (SEQ ID | CAGACTGAA (SEQ ID |
| | | NO: 226) | NO: 227) |
| p30_CWSV00129 | 3 | CATGGGGATGCTTGGTGC | TGTTGGCTCCTATGTCCATT |
| | | AGGCTTCTATTT (SEQ ID | TGACATTT (SEQ ID NO: 229) |
| | | NO: 228) | |
| p30_CWSV00145 | 3 | CATGGGAAATCCCTCAGTC | TGCCTGTGTATATACAGCAT |
| | | CGGAAGTCTAGAAC (SEQ | GTATGATGTACATTT (SEQ |
| | | ID NO: 230) | ID NO: 231) |
| p30_CWSV00148 | 3 | CATGGCACATGCAAATAA | GTATCGAAGTATATAAACTG |
| | | TGCAATCATATATGGCTTA | ATGGTTCACAGAGA (SEQ ID |
| | | AT (SEQ ID NO: 232) | NO: 233) |
| p30_CWSV00149 | 3 | CATGTACCATAACTTAATG | CACGCAACCGTAGGTGTGTA |
| | | CCATATTTGCCATAGTTGG | TTCCAG (SEQ ID NO: 235) |
| | | C (SEQ ID NO: 234) | |
| p30_CWSV00155 | 3 | CATGAAATGTAAACCTTGT | TTTGTCTCTGCTTACATTTGG |
| | | CTAAATCTGGGATCTTC | CTCTCTGTAATTT (SEQ ID |
| | | (SEQ ID NO: 236) | NO: 237) |
| p30_CWSV00172 | 3 | CATGAAAGCTCACAATTA | CCTGTATTTCTGTGTCTTCAC |
| | | CAAGTGCTAAGTCTTCAGA | ATGGCATTCTC (SEQ ID |
| | | AA (SEQ ID NO: 238) | NO: 239) |
| p30_CWSV00173 | 3 | CATGCTAAATCCTTTCCCG | GCTGCATAAATGTCTTCTTT |
| | | TTAGGGACTGTTCTGATC | AGAGAAGTGTCTTT (SEQ ID |
| | | (SEQ ID NO: 240) | NO: 241) |
| p30_CWSV00175 | 3 | CATGAATGGTGGAGGTAG | CTTGAACTCCTGGGCTCAAG |
| | | CAGCATTGCAATC (SEQ ID | CGATC (SEQ ID NO: 243) |
| | | NO: 242) | |
| p30_CWSV00185 | 3 | CATGTTACTCATTCTTAAG | CATCATCCCACCAAGTTAGC |
| | | CACTAGGTGTGATTCTAAA | TCCAA (SEQ ID NO: 245) |
| | | T (SEQ ID NO: 244) | |
| p30_CWSV00193 | 3 | CATGTTAAGGCATAGCTTA | TCCCAGGTAATGCTGATGTT |
| | | GCAGTCCACCAAAGAT | GCCAGT (SEQ ID NO: 247) |
| | | (SEQ ID NO: 246) | |
| p30_CWSV00194 | 3 | CATGAATGCTTAAGATATG | CAAGCTCAAGATTTCTGTTA |
| | | TACCCAGCATACCTTAAAT | GTTACCAGTTTCTTT (SEQ ID |
| | | G (SEQ ID NO: 248) | NO: 249) |
| p30_CWSV00196 | 3 | CATGAAAGTCACACTGTTG | GGGCTCTGGTGTGACGAAGT |
| | | CTAACAAGCTGATAAA | CATTG (SEQ ID NO: 251) |
| | | (SEQ ID NO: 250) | |
| p30_CWSV00197 | 3 | CATGCTGCACTCCAGCCTG | TAGGAGTGCTTCTCTGATGA |
| | | GGCG (SEQ ID NO: 252) | TGTTCCACC (SEQ ID NO: 253) |
| p30_CWSV00198 | 3 | CATGCATATTATATCTTTG | TTATGGTTATTACTTGACAG |
| | | CTTGCCTAGTTTCTCATGT | AGGGCAGT (SEQ ID NO: 255) |
| | | T (SEQ ID NO: 254) | |
| p30_CWSV00215 | 3 | CATGATGGTAGCATGACTT | TTGGTGCTTTATGGTTTCATT |
| | | CCTGGCCTCTAGT (SEQ ID | CTGAACAC (SEQ ID NO: 257) |
| | | NO: 256) | |
| p30_CWSV00216 | 3 | CATGTTGGCAATGGCATTT | ATGAGATAGATGACAGAAC |
| | | GGTTTCCTCCA (SEQ ID | TGACACCTAG (SEQ ID |
| | | NO: 258) | NO: 259) |
| p30_CWSV00218 | 3 | CATGTAGTCTCAGCTACTT | TCCTGGCATTTGGGAAGCTT |
| | | GGGAAGCTGAGG (SEQ ID | GCAC (SEQ ID NO: 261) |
| | | NO: 260) | |
| p30_CWSV00227 | 3 | CATGTAAGTGGAGATAAG | GGAGGAGGGCATTTGAGGA |
| | | CTTGTATGTCTTATATGG | GTCTCTATG (SEQ ID NO: 263) |
| | | (SEQ ID NO: 262) | |
| p30_CWSV00233 | 3 | CATGTTTATTGGAGGGCCA | AGTGTGATAGTGTGCGCCTG |
| | | GGTAATAGCTTTGCA (SEQ | TCAACT (SEQ ID NO: 265) |
| | | ID NO: 264) | |
| p30_CWSV00250 | 3 | CATGTGAAAGTCCACCTGT | ACCTAGCCTTTGCTCTAGCA |
| | | GAGTTGCAGAACT (SEQ ID | CCGG (SEQ ID NO: 267) |
| | | NO: 266) | |
| p30_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p30_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p30_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p30_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p30_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

P38

| | | | |
|---|---|---|---|
| p38_CWSV00001 | 3 | CATGGACGGAGTTTCACTC | AGAGGTGGCAATGGGCCGA |
| | | TTTCGCCCAG (SEQ ID | GAT (SEQ ID NO: 467) |
| | | NO: 466) | |
| p38_CWSV00006 | 3 | CATGAAAGTCTCAAGTAG | ACTGCTGGCTGGAAACAGA |
| | | TCGGCATATCTGTTT (SEQ | GGCTAA (SEQ ID NO: 469) |
| | | ID NO: 468) | |
| p38_CWSV00011 | 3 | CATGGTGATAATTTAAGGC | CATGCACTTGTAGTTCCAGG |
| | | AGTTTCTCATCTGCCTTC | TACTAGGGAG (SEQ ID |
| | | (SEQ ID NO: 470) | NO: 471) |
| p38_CWSV00014 | 3 | CATGAAATTAGCCGGACA | GTGCGCGCCTGTAATCCCAG |
| | | TGGTGGTGCATG (SEQ ID | C (SEQ ID NO: 473) |
| | | NO: 472) | |
| p38_CWSV00016 | 3 | CATGAAATTCCCAAGTCAC | AAAGGGACTCCAAGTATTGT |
| | | TTTGTTCAGAAATATTTCA | ACGATGAAA (SEQ ID |
| | | G (SEQ ID NO: 474) | NO: 475) |
| p38_CWSV00019 | 3 | CATGTCCTTAGACTTTCCT | CACTGAGAGGCCTAGAAGC |
| | | TGCTTTCAGTAACCTTGAG | AGTGACC (SEQ ID NO: 477) |
| | | (SEQ ID NO: 476) | |
| p38_CWSV00022 | 3 | CATGTTTAGTTAGATTGTC | CCACAAAGTGCAAAGTTTCA |
| | | TTCTTATTGTTGAGTTACA | TGCAACTT (SEQ ID NO: 479) |
| | | G (SEQ ID NO: 478) | |
| p38_CWSV00024 | 3 | CATGAATTACATTTAATGC | GTTCTATCCATTAAGATCAT |
| | | CTGTTGATGAGGGCTCAA | GCTCACTAATTTAGA (SEQ |
| | | G (SEQ ID NO: 480) | ID NO: 481) |
| p38_CWSV00029 | 3 | CATGTTTAGTAGCATGGCC | GACCAGCCTAGGCAACATA |
| | | ATGTGCTTT (SEQ ID | GTGAGACC (SEQ ID NO: 483) |
| | | NO: 482) | |
| p38_CWSV00037 | 3 | CATGGCCAAGGCCCACCC | CAGGATTCCGTAAGCCAGGT |
| | | TGCAG (SEQ ID NO: 484) | TT (SEQ ID NO: 485) |
| p38_CWSV00040 | 3 | CATGCAAATCAAATCCCA | TTCTCATCTTCCCAGCTGAC |
| | | GCTGAGCCACTCTG (SEQ | ATTT (SEQ ID NO: 487) |
| | | ID NO: 486) | |
| p38_CWSV00045 | 3 | CATGAATCCACCCACCTAG | GACTCAATTTATGTTGTTTC |
| | | AATTCTGTACTCAGC (SEQ | CATTCCAGTTT (SEQ ID |
| | | ID NO: 488) | NO: 489) |
| p38_CWSV00050 | 3 | CATGCCACCACGCCCAGCT | AGTATGAAGCCCACCTAGAT |
| | | AATTT (SEQ ID NO: 490) | TCATTGTGAGG (SEQ ID |
| | | | NO: 491) |
| p38_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p38_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p38_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p38_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p38_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

P39

| | | | |
|---|---|---|---|
| p39_CWSV00002 | 3 | CATGATTTATTTACAACTG | CGATCTCGGCTCACTGCAAC |
| | | TGATTGGACAAGAAGAAT | CTC (SEQ ID NO: 493) |
| | | TC (SEQ ID NO: 492) | |
| p39_CWSV00005 | 3 | CATGATTATTTATATGATC | CAGGAGACCTCTCTGAGTAG |
| | | TTCACCTGTGCAATTCCT | TTGACAATT (SEQ ID NO: 495) |
| | | (SEQ ID NO: 494) | |
| p39_CWSV00009 | 3 | CATGATCTCTCAAGTGAGA | GACTTCAGAAGCTAGTTACT |
| | | GGATTCTTCCTTCCTGA | GAAGGAATACT (SEQ ID |
| | | (SEQ ID NO: 496) | NO: 497) |
| p39_CWSV00010 | 3 | CATGAATAAGTCTCTCAGT | ATGTAATGACGGACTTAGTG |
| | | CATTATATTAGCTTCTAAC | GCAATAAA (SEQ ID NO: 499) |
| | | A (SEQ ID NO: 498) | |
| p39_CWSV00017 | 3 | CATGGGGAAGAGGTCAGG | AATCCTCCCAGCACTTTGGG |
| | | ATTTGCACCTATATCTG | AGGC (SEQ ID NO: 501) |
| | | (SEQ ID NO: 500) | |
| p39_CWSV00019 | 3 | CATGCTCAGGACTGAAGG | GCTATTCGGGAGGCTGAGG |
| | | GTTTCCCAGGAC (SEQ ID | CAG (SEQ ID NO: 503) |
| | | NO: 502) | |
| p39_CWSV00033 | 3 | CATGATGTTTAGTTATGCC | TATGTCTACTCTGTAGCATT |
| | | CTATGCAGATGTCTTGGT | TGGTGGTTCATCC (SEQ ID |
| | | (SEQ ID NO: 504) | NO: 505) |
| p39_CWSV00034 | 3 | CATGGGGACGGTACGGTG | CAGATAATCAGCATTAATAA |
| | | CGCG (SEQ ID NO: 506) | TATGTCCAGCATGAC (SEQ |
| | | | ID NO: 507) |
| p39_CWSV00039 | 3 | CATGACATAAAGACTTTGC | CAATAGTGGGACAGGCATA |
| | | TGATGATATATTAACAAAT | GCATAGACATTCTC (SEQ ID |
| | | G (SEQ ID NO: 508) | NO: 509) |
| p39_CWSV00046 | 3 | CATGACGGCATATCCTGCT | ATGGGCACTATTGCTTTCTA |
| | | CACCGACTT (SEQ ID | AATGCACAA (SEQ ID |
| | | NO: 510) | NO: 511) |
| p39_CWSV00048 | 3 | CATGTTTCCTGTGCTGAAG | GGAAGTTCATGCCTAAGAA |
| | | TGACCCTTTGGT (SEQ ID | ACATGTAGCA (SEQ ID |
| | | NO: 512) | NO: 513) |
| p39_CWSV00060 | 3 | CATGTTAGGTCTTAGATTT | TCTCGGCTCACTGCAAGCTC |
| | | AAGTTCCTGATCCATTT | CG (SEQ ID NO: 515) |
| | | (SEQ ID NO: 514) | |
| p39_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p39_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p39_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p39_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p39_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

P54

| | | | |
|---|---|---|---|
| p54_CWSV00009 | 3 | CATGGGGTGTGGTGGCTC | CATGCTTGTACCTGGTGTTC |
| | | ATGGCTG (SEQ ID NO: 268) | GGTTAT (SEQ ID NO: 113) |
| p54_CWSV00010 | 3 | CATGCAGAAGAGTGTGTT | CATGCCAGCCCAAGATGGA |
| | | GCCTTCCACTGTTC (SEQ | TGAAT (SEQ ID NO: 114) |
| | | ID NO: 269) | |
| p54_CWSV00023 | 3 | CATGCAAACTTGCTTAGTT | CATGGCGGTGGAAGTCATTC |
| | | GTTTACCTGTGATATTGGC | TGATA (SEQ ID NO: 115) |
| | | T (SEQ ID NO: 270) | |
| p54_CWSV00032 | 3 | CATGACCATTTAATCCAAG | CATGGAGGGCCACAGATTG |
| | | CTACCCGCTATGTCG (SEQ | GTATT (SEQ ID NO: 116) |
| | | ID NO: 271) | |
| p54_CWSV00035 | 3 | CATGGCTGAACTGGCCTA | CATGACTGAAAGGTCAGATT |
| | | ACCTGAATGTATGTACA | CAGGAG (SEQ ID NO: 117) |
| | | (SEQ ID NO: 272) | |
| p54_CWSV00042 | 3 | CATGGTATTTGTATGGTCA | GGTTCAACTGAAGTGCCAGC |
| | | TACACGAGGAACTGCTGT | CTGC (SEQ ID NO: 274) |
| | | A (SEQ ID NO: 273) | |
| p54_CWSV00048 | 3 | CATGAAACTCCTGACCTCA | CCTCAAAGTGTCTGCAGAGC |
| | | AGTGATCCACCTG (SEQ ID | AGGAATC (SEQ ID NO: 276) |
| | | NO: 275) | |
| p54_CWSV00057 | 3 | CATGTTATGGCTCAGGATA | GAAGGATCAGAGTACCAGC |
| | | AATTCCTACATGTAGAATT | ATCCGTCTTC (SEQ ID |
| | | C (SEQ ID NO: 277) | NO: 278) |
| p54_CWSV00095 | 3 | CATGGGGTTTCACCATGTT | CAGTTTATATTCCAGATAGG |
| | | GGCCAGGATG (SEQ ID | CAGGAGGTT (SEQ ID |
| | | NO: 279) | NO: 280) |
| p54_CWSV00132 | 3 | CATGAATACCAGTAAGAG | CTGTGCCCATTTCACGCCCT |
| | | CTTGAACTTTGTAAGTTAA | GG (SEQ ID NO: 282) |
| | | AC (SEQ ID NO: 281) | |
| p54_CWSV00144 | 3 | CATGTGTGCAGCAGACTTC | TTTGTGAAAGGCAAATGATG |
| | | TGCCTGGA (SEQ ID | CATGACAG (SEQ ID NO: 284) |
| | | NO: 283) | |
| p54_CWSV00162 | 3 | CATGTACAGTATTAGGCAC | GAAAGGTTAAGCAACTTGC |
| | | CTGACTGGGTAAA (SEQ ID | CCATGGTCAA (SEQ ID |
| | | NO: 285) | NO: 286) |
| p54_CWSV00185 | 3 | CATGGTGGCACATGCCTGT | AGGCCCAGCCATCTCACTGC |
| | | AATCCCAGC (SEQ ID | A (SEQ ID NO: 288) |
| | | NO: 287) | |
| p54_CWSV00191 | 3 | CATGGATAATCTGGGTACC | GGGCCCATAGGATTTCTGGA |
| | | AGCCTTAAGGAAGGC (SEQ | CTGAAATCA (SEQ ID |
| | | ID NO: 289) | NO: 290) |
| p54_CWSV00206 | 3 | CATGTTGAGCTAAGCCAGT | GAGCGATTCTCCTGCCTAGG |
| | | CCATCAAGACTGG (SEQ ID | CCT (SEQ ID NO: 292) |
| | | NO: 291) | |
| p54_CWSV00230 | 3 | CATGTGCCTCGGCCTCCCA | ACAGTAACCAGATCTCAGCC |
| | | AGTAGC (SEQ ID NO: 293) | AATCAGAGG (SEQ ID |
| | | | NO: 294) |
| p54_CWSV00240 | 3 | CATGAAATTAGCTGGTGG | GGTTACAGTGAGCCATGATT |
| | | CAGGCACCTGTA (SEQ ID | GTGCCG (SEQ ID NO: 296) |
| | | NO: 295) | |
| p54_CWSV00260 | 3 | CATGATTTCGAGAAATAA | TAGGGCATTTCACATGGGCT |
| | | CTTCCGATTTATTTCAGAT | CAGAAACA (SEQ ID NO: 298) |
| | | AG (SEQ ID NO: 297) | |
| p54_CWSV00266 | 3 | CATGAAGCCAAGGCTGAG | AAACTTCCAGACTGATTCCA |
| | | CCAAGTCAG (SEQ ID | AAGTCCAAG (SEQ ID |
| | | NO: 299) | NO: 300) |
| p54_CWSV00268 | 3 | CATGTCATTGATTCCAGTG | GGTGACAGAGGGAGACTCT |
| | | CGGTCCTAAA (SEQ ID | GTCACAAA (SEQ ID NO: 302) |
| | | NO: 301) | |
| p54_CWSV00281 | 3 | CATGCGCCCGGACAGTGG | TCTTCCTGTGTTACCTAGGC |
| | | AGACG (SEQ ID NO: 303) | TGGTCTCA (SEQ ID NO: 304) |
| p54_CWSV00305 | 3 | CATGTGTCCCACTGGAGA | GCTAGGATTACAGGCGTGA |
| | | ACCCTGACTAATACA (SEQ | GCCACTAT (SEQ ID NO: 306) |
| | | ID NO: 305) | |
| p54_CWSV00320 | 3 | CATGCTTCTACTTGTTGGA | AGTCATGGAGGTAGAGCCTT |
| | | CTTCTGATAGGGTCCAAAG | CTTGAAAGG (SEQ ID |
| | | (SEQ ID NO: 307) | NO: 308) |
| p54_CWSV00335 | 3 | CATGCCTCACAGGTAGGC | GCCCTGAGGCTCACGTCACT |
| | | GTGAGGATCAG (SEQ ID | CA (SEQ ID NO: 310) |
| | | NO: 309) | |
| p54_CWSV00339 | 3 | CATGCTCAACATGAAGAC | ATGAAGCTAGGATTATTAAC |
| | | AACAGGGATGAGGAGC | CTAAGGGTCACTTGT (SEQ |
| | | (SEQ ID NO: 311) | ID NO: 312) |
| p54_CWSV00360 | 3 | CATGGTCGCTCAGGCTGGT | AAACACAGTCCCTGTCCTTA |
| | | GTGCAG (SEQ ID NO: 313) | CAGAGCTC (SEQ ID NO: 314) |
| p54_CWSV00369 | 3 | CATGGGCTTACATCTGTAA | ACTGCCTCAGTTAACCAGCA |
| | | TCCCAGCACTTTGAGA | GACTTCA (SEQ ID NO: 316) |
| | | (SEQ ID NO: 315) | |
| p54_CWSV00374 | 3 | CATGCCTCCCACCTTGGCC | TCCTGGGCTCAAGCAATCCT |
| | | TCCCAA (SEQ ID NO: 317) | CCTG (SEQ ID NO: 318) |
| p54_CWSV00416 | 3 | CATGATGGATCCTGGTACC | GGAGCTTGTACAGCAGCTA |
| | | CAGAGGGAATACAA (SEQ | AGGTGAG (SEQ ID NO: 320) |
| | | ID NO: 319) | |
| p54_CWSV00426 | 3 | CATGGACTCAAACTCCTGG | TCCCTGAGATCAAGTGATTG |
| | | GCTCAAGTGATCC (SEQ ID | TCCCATCTT (SEQ ID NO: 322) |
| | | NO: 321) | |
| p54_CWSV00428 | 3 | CATGCCCATGGACTCGTAA | AATACTCTGGATGATCTTCA |
| | | TTGGTTTCCTGGA (SEQ ID | GGACATGTT (SEQ ID NO: 324) |
| | | NO: 323) | |
| p54_CWSV00432 | 3 | CATGGTGATAGCATGCCTC | GGAGGTGGAGGCGGGAGGA |
| | | TGTAGTCCCAGC (SEQ ID | TT (SEQ ID NO: 326) |
| | | NO: 325) | |
| p54_CWSV00435 | 3 | CATGAATGAGTGAGTGAC | AACCCTTCATGAACGTGAGG |
| | | GAGGCTGCTGG (SEQ ID | AAGCTGA (SEQ ID NO: 328) |
| | | NO: 327) | |
| p54_CWSV00447 | 3 | CATGTCTTAGCCATTCAGA | AATACTGTAGCCTTCCTAGG |
| | | TCAATCCCAGACAGACTA | GCTCTCACA (SEQ ID NO: 330) |
| | | (SEQ ID NO: 329) | |
| p54_CWSV00449 | 3 | CATGTACACCTTAACCTAG | CTGCCACAGCCTCCCATGTA |
| | | CAATGCTTTCTCTTCTGAG | GCT (SEQ ID NO: 332) |
| | | (SEQ ID NO: 331) | |
| p54_CWSV00461 | 3 | CATGTCTGCCTCAGCCTCC | CTAAGGAGAGACTGCTTTCT |
| | | CATGTAGC (SEQ ID | GATGCCTACC (SEQ ID |
| | | NO: 333) | NO: 334) |
| p54_CWSV00475 | 3 | CATGCATGCATTGCAACGC | CAGTGCTATCCAGCCCAGAT |
| | | AGTGGTGC (SEQ ID | ACTATGCTTT (SEQ ID |
| | | NO: 335) | NO: 336) |
| p54_CWSV00512 | 3 | CATGCTGCGAACACTTAAT | AGGATGGTATCGATCTCTTC |
| | | GCAGAGCTTGATACC (SEQ | ACCTCGTGA (SEQ ID NO: 338) |
| | | ID NO: 337) | |
| p54_CWSV00541 | 3 | CATGCTTCCAGCTAGTTTC | AAGTTGCAAACTGCATTGAG |
| | | TAGAGGTCTTGTCTGGA | ATAGACAATTAAA (SEQ ID |
| | | (SEQ ID NO: 339) | NO: 340) |
| p54_CWSV00574 | 3 | CATGCTAGGTGACAGCATT | TGGCTAACGCCTGTGATCCC |
| | | GAGCAGGGCT (SEQ ID | AGC (SEQ ID NO: 342) |
| | | NO341:) | |
| p54_CWSV00611 | 3 | CATGGTTCAAGAGCGATG | CTCGCTGCAACTTCCACCCG |
| | | GCTCTCGAATAAA (SEQ ID | C (SEQ ID NO: 344) |
| | | NO: 343) | |
| p54_CWSV00624 | 3 | CATGGGGCATCACCACAG | TTTCCTGTTATTTCTATAAG |
| | | GCTGAGGA (SEQ ID | ATGCTTCCTCTGATT (SEQ ID |
| | | NO: 345) | NO: 346) |
| p54_CWSV00647 | 3 | CATGGAGGTTGCATTGAG | GAGAATGCAGCCCAGTAGG |
| | | CCAAGATCATGGC (SEQ ID | TCTCAGC (SEQ ID NO: 348) |
| | | NO: 347) | |
| p54_DNAIA_short1 | 3 | CATGCTTGTACCTGGTGTT | GTAGGCTGGGTAGCCAAAT |
| | | CGGTTAT (SEQ ID NO: 113) | C (SEQ ID NO: 2) |
| p54_DNAIA_short2 | 3 | CATGCCAGCCCAAGATGG | CTCTTCTTTCCTTTCAGCAAC |
| | | ATGAAT (SEQ ID NO: 114) | AC (SEQ ID NO: 4) |
| p54_DNAIA_short3 | 3 | CATGGCGGTGGAAGTCAT | TCTCTCTCTCTCCAGTGAAC |
| | | TCTGATA (SEQ ID NO: 115) | AA (SEQ ID NO: 6) |
| p54_DNAIA_short4 | 3 | CATGGAGGGCCACAGATT | GATCACTTAGCTCAGGAAAC |
| | | GGTATT (SEQ ID NO: 116) | AAAC (SEQ ID NO: 8) |
| p54_DNAIA_short5 | 3 | CATGACTGAAAGGTCAGA | TACCGGTTTGTGTCCAAGAG |
| | | TTCAGGAG (SEQ ID | (SEQ ID NO: 10) |
| | | NO: 117) | |

*Tag sequence is indicated with underline. SV targets and primers are indicated with “CWSV.” Control targets and primers are indicated with “DNAIA.”

TABLE 4
Targets, tag, and, primer sequences for three consecutive experiments using three
different tags (underlined) on a single template (HCC1954v2) as shown in FIG. 4B.*

| Target | Tag | Forward Primer Sequence | Reverse Primer Sequence |
|---|---|---|---|
| HCC1954v2_CWSV00079 | 2 | CGTATGAGGCTCGACA | GCCTGTCGTGCAGCCATG |
| | | GGTACCATATAGCCT | AGTTAG (SEQ ID NO: 12) |
| | | (SEQ ID NO: 349) | |
| HCC1954v2_CWSV00085 | 2 | CGTAGACTCCAACCAT | AAAGGAGAACTATGTGA |
| | | TGGTATAATCTGAAAG | GCTAATGCATTT (SEQ ID |
| | | TTGTCT (SEQ ID NO: 350) | NO: 14) |
| HCC1954v2_CWSV00096 | 2 | CGTACTGCCTGACAAC | CTGTATATCTGGCTAGCC |
| | | GGACTGAAA (SEQ ID | ATATGCAGAAA (SEQ ID |
| | | NO: 351) | NO: 16) |
| HCC1954v2_CWSV00142 | 2 | CGTATCGGCTATTTCTA | CAATCAGAAGACTGCTA |
| | | ACAGCTCACATGTTTAC | GGGTGGCTGTATT (SEQ |
| | | AT (SEQ ID NO: 352) | ID NO: 18) |
| HCC1954v2_CWSV00166 | 2 | CGTATTCCATAAGTGCC | GAGTGTATAGACTAGAA |
| | | AGCTGGCTGAGAAATA | GTACAGCCATATAGTT |
| | | (SEQ ID NO: 353) | (SEQ ID NO: 20) |
| HCC1954v2_CWSV00203 | 2 | CGTATCTGCTCCCAGGT | CCCTGTTGCCCAGGCTAG |
| | | TCAAGTGATTCTCAAG | AGTG (SEQ ID NO: 22) |
| | | (SEQ ID NO: 354) | |
| HCC1954v2_CWSV00281 | 2 | CGTATCCGAGAGCTCA | GAGCGAGCTCAAGATAG |
| | | CAGCTTGGCT (SEQ ID | AGGCAGCTAA (SEQ ID |
| | | NO: 355) | NO: 24) |
| HCC1954v2_CWSV00291 | 2 | CGTAGTTTGATGGAATT | TCCACATCACGGGAGCA |
| | | CACCAGTGTGTTT (SEQ | GCTG (SEQ ID NO: 26) |
| | | ID NO: 356) | |
| HCC1954v2_CWSV00362 | 2 | CGTAATTTAGAGAGCC | GTCCAGCTGAGTGGAAC |
| | | TGTCACAAATGTCCAT | ATTGAAAGAACA (SEQ ID |
| | | ATGAGAA (SEQ ID | NO: 28) |
| | | NO: 357) | |
| HCC1954v2_CWSV00739 | 2 | CGTAGAGAGGAGAGAG | TAGGAGGAGCCCATTGG |
| | | TGCAGGGTCAA (SEQ ID | CC (SEQ ID NO: 30) |
| | | NO: 358) | |
| HCC1954v2_CWSV00742 | 2 | CGTAACATTATTGTTAA | GCAGGAGAATGGCATGA |
| | | CAAGGGTTGCTTCTGG | ACCCGG (SEQ ID NO: 32) |
| | | GAAGA (SEQ ID NO: 359) | |
| HCC1954v2_CWSV00743 | 2 | CGTACCCTGGCACCTA | AGATGTGGCTGAACTTTA |
| | | GTGGGTCCATAC (SEQ | AATGCATATT (SEQ ID |
| | | ID NO: 360) | NO: 34) |
| HCC1954v2_CWSV00744 | 2 | CGTAGCACTCCAGAGA | AAAGGGCCAGGCCTGAG |
| | | CTCTGTTTCTAAA (SEQ | TGGT (SEQ ID NO: 36) |
| | | ID NO: 361) | |
| HCC1954v2_CWSV00791 | 2 | CGTACAGGCATGGTAG | CTCCGGCACTGGGCAGA |
| | | CGCGCCT (SEQ ID | GGTA (SEQ ID NO: 38) |
| | | NO: 362) | |
| HCC1954v2_CWSV00841 | 2 | CGTAGGAAGCCTCATA | TCAGTGCTAGCTGATAAC |
| | | ATCATGGTGGAAGGCA | TCTGACTTT (SEQ ID |
| | | (SEQ ID NO: 363) | NO: 40) |
| HCC1954v2_CWSV00852 | 2 | CGTACATGTTTACCGAT | CACTGCATGTTCCCAGTC |
| | | GAAGCTGCAAA (SEQ ID | ATAGGTGG (SEQ ID |
| | | NO: 364) | NO: 42) |
| HCC1954v2_CWSV00856 | 2 | CGTAAGGAAGCTCAGC | AGACCCATCTCACATGTA |
| | | TATACCTGTTACTGATA | GACACA (SEQ ID NO: 44) |
| | | ATTT (SEQ ID NO: 365) | |
| HCC1954v2_CWSV01009 | 2 | CGTACCCATGCAGGCC | CTTCTCCAAGTCTCTACA |
| | | TAGGCTAGTGT (SEQ ID | CTTGGCCACAA (SEQ ID |
| | | NO: 366) | NO: 46) |
| HCC1954v2_CWSV01021 | 2 | CGTAACCTAAAGGACA | AGGATGACGCCTGGCTT |
| | | CATCTCAGTACATTT | GGAGAAT (SEQ ID NO: 48) |
| | | (SEQ ID NO: 367) | |
| HCC1954v2_CWSV01028 | 2 | CGTAACTGAAGGCTCT | AGCAACAAAGATTATCC |
| | | ATGTGCAGGAACTCC | TAAGACTTCTGGAGGAA |
| | | (SEQ ID NO: 368) | G (SEQ ID NO: 50) |
| HCC1954v2_CWSV01070 | 2 | CGTACCTCCCAAAGTG | AGGAGGGAGTGACAGGA |
| | | CTGGGATTATAGGCA | TGTGATCTG (SEQ ID |
| | | (SEQ ID NO: 369) | NO: 52) |
| HCC1954v2_CWSV01075 | 2 | CGTAAACTGTTCTTCCA | AGCCTGGCCAACACAGT |
| | | GGCCTGATTT (SEQ ID | GAAACC (SEQ ID NO: 54) |
| | | NO: 370) | |
| HCC1954v2_CWSV01077 | 2 | CGTACCCATCCTGGCA | TGACCTGTTCTTGGGTAG |
| | | CGTACTGCTACT (SEQ | CACTATTGGT (SEQ ID |
| | | ID NO: 371) | NO: 56) |
| HCC1954v2_CWSV01100 | 2 | CGTAAGGCACACTCAG | AAAGGGATAGCAGTTTC |
| | | GCTTTACGATTCTTGTT | CTGCTGTCCATAG (SEQ |
| | | TA (SEQ ID NO: 372) | ID NO: 58) |
| HCC1954v2_CWSV01109 | 2 | CGTAATTCTCCCTGCAA | CCCAGGCTGCTGGTCATG |
| | | ATTGTTTGACTCTAGAA | GAAA (SEQ ID NO: 60) |
| | | ATAGC (SEQ ID NO: 373) | |
| HCC1954v2_CWSV01130 | 2 | CGTAGGGTAGTAGCGA | CACAGGGTTTATCTCCTC |
| | | TCTGGGTTACCCAG | AGACAAA (SEQ ID NO: 62) |
| | | (SEQ ID NO: 374) | |
| HCC1954v2_CWSV01177 | 2 | CGTACATTGACCCACC | GCGGACGCCATGGAGCT |
| | | GGTCATTCAGGAG (SEQ | (SEQ ID NO: 64) |
| | | ID NO: 375) | |
| HCC1954v2_CWSV01529 | 2 | CGTACTGAGGGTTATC | AACACATTAGTGATGTCT |
| | | CAAGTGACCCATTGCA | CAATGCCATCC (SEQ ID |
| | | (SEQ ID NO: 376) | NO: 66) |
| HCC1954v2_DNAIA_short1 | 2 | CGTACTTGTACCTGGTG | GTAGGCTGGGTAGCCAA |
| | | TTCGGTTAT (SEQ ID | ATC (SEQ ID NO: 2) |
| | | NO: 377) | |
| HCC1954v2_DNAIA_short2 | 2 | CGTACCAGCCCAAGAT | CTCTTCTTTCCTTTCAGC |
| | | GGATGAAT (SEQ ID | AACAC (SEQ ID NO: 4) |
| | | NO: 378) | |
| HCC1954v2_DNAIA_short3 | 2 | CGTAGCGGTGGAAGTC | TCTCTCTCTCTCCAGTGA |
| | | ATTCTGATA (SEQ ID | ACAA (SEQ ID NO: 6) |
| | | NO: 379) | |
| HCC1954v2_DNAIA_short4 | 2 | CGTAGAGGGCCACAGA | GATCACTTAGCTCAGGA |
| | | TTGGTATT (SEQ ID | AACAAAC (SEQ ID NO: 8) |
| | | NO: 380) | |
| HCC1954v2_DNAIA_short5 | 2 | CGTAACTGAAAGGTCA | TACCGGTTTGTGTCCAAG |
| | | GATTCAGGAG (SEQ ID | AG (SEQ ID NO: 10) |
| | | NO: 381) | |
| HCC1954v2_CWSV00079 | 3 | GTACTGAGGCTCGACA | GCCTGTCGTGCAGCCATG |
| | | GGTACCATATAGCCT | AGTTAG (SEQ ID NO: 12) |
| | | (SEQ ID NO: 382) | |
| HCC1954v2_CWSV00085 | 3 | GTACGACTCCAACCAT | AAAGGAGAACTATGTGA |
| | | TGGTATAATCTGAAAG | GCTAATGCATTT (SEQ ID |
| | | TTGTCT (SEQ ID NO: 383) | NO: 14) |
| HCC1954v2_CWSV00096 | 3 | GTACCTGCCTGACAAC | CTGTATATCTGGCTAGCC |
| | | GGACTGAAA (SEQ ID | ATATGCAGAAA (SEQ ID |
| | | NO: 384) | NO: 16) |
| HCC1954v2_CWSV00142 | 3 | GTACTCGGCTATTTCTA | CAATCAGAAGACTGCTA |
| | | ACAGCTCACATGTTTAC | GGGTGGCTGTATT (SEQ |
| | | AT (SEQ ID NO: 385) | ID NO: 18) |
| HCC1954v2_CWSV00166 | 3 | GTACTTCCATAAGTGCC | GAGTGTATAGACTAGAA |
| | | AGCTGGCTGAGAAATA | GTACAGCCATATAGTT |
| | | (SEQ ID NO: 386) | (SEQ ID NO: 20) |
| HCC1954v2_CWSV00203 | 3 | GTACTCTGCTCCCAGGT | CCCTGTTGCCCAGGCTAG |
| | | TCAAGTGATTCTCAAG | AGTG (SEQ ID NO: 22) |
| | | (SEQ ID NO: 387) | |
| HCC1954v2_CWSV00281 | 3 | GTACTCCGAGAGCTCA | GAGCGAGCTCAAGATAG |
| | | CAGCTTGGCT (SEQ ID | AGGCAGCTAA (SEQ ID |
| | | NO: 388) | NO: 24) |
| HCC1954v2_CWSV00291 | 3 | GTACGTTTGATGGAATT | TCCACATCACGGGAGCA |
| | | CACCAGTGTGTTT (SEQ | GCTG (SEQ ID NO: 26) |
| | | ID NO: 389) | |
| HCC1954v2_CWSV00362 | 3 | GTACATTTAGAGAGCC | GTCCAGCTGAGTGGAAC |
| | | TGTCACAAATGTCCAT | ATTGAAAGAACA (SEQ ID |
| | | ATGAGAA (SEQ ID | NO: 28) |
| | | NO: 390) | |
| HCC1954v2_CWSV00739 | 3 | GTACGAGAGGAGAGAG | TAGGAGGAGCCCATTGG |
| | | TGCAGGGTCAA (SEQ ID | CC (SEQ ID NO: 30) |
| | | NO: 391) | |
| HCC1954v2_CWSV00742 | 3 | GTACACATTATTGTTAA | GCAGGAGAATGGCATGA |
| | | CAAGGGTTGCTTCTGG | ACCCGG (SEQ ID NO: 32) |
| | | GAAGA (SEQ ID NO: 392) | |
| HCC1954v2_CWSV00743 | 3 | GTACCCCTGGCACCTA | AGATGTGGCTGAACTTTA |
| | | GTGGGTCCATAC (SEQ | AATGCATATT (SEQ ID |
| | | ID NO: 393) | NO: 34) |
| HCC1954v2_CWSV00744 | 3 | GTACGCACTCCAGAGA | AAAGGGCCAGGCCTGAG |
| | | CTCTGTTTCTAAA (SEQ | TGGT (SEQ ID NO: 36) |
| | | ID NO: 394) | |
| HCC1954v2_CWSV00791 | 3 | GTACCAGGCATGGTAG | CTCCGGCACTGGGCAGA |
| | | CGCGCCT (SEQ ID | GGTA (SEQ ID NO: 38) |
| | | NO: 395) | |
| HCC1954v2_CWSV00841 | 3 | GTACGGAAGCCTCATA | TCAGTGCTAGCTGATAAC |
| | | ATCATGGTGGAAGGCA | TCTGACTTT (SEQ ID |
| | | (SEQ ID NO: 396) | NO: 40) |
| HCC1954v2_CWSV00852 | 3 | GTACCATGTTTACCGAT | CACTGCATGTTCCCAGTC |
| | | GAAGCTGCAAA (SEQ ID | ATAGGTGG (SEQ ID |
| | | NO: 397) | NO: 42) |
| HCC1954v2_CWSV00856 | 3 | GTACAGGAAGCTCAGC | AGACCCATCTCACATGTA |
| | | TATACCTGTTACTGATA | GACACA (SEQ ID NO: 44) |
| | | ATTT (SEQ ID NO: 398) | |
| HCC1954v2_CWSV01009 | 3 | GTACCCCATGCAGGCC | CTTCTCCAAGTCTCTACA |
| | | TAGGCTAGTGT (SEQ ID | CTTGGCCACAA (SEQ ID |
| | | NO: 399) | NO: 46) |
| HCC1954v2_CWSV01021 | 3 | GTACACCTAAAGGACA | AGGATGACGCCTGGCTT |
| | | CATCTCAGTACATTT | GGAGAAT (SEQ ID NO: 48) |
| | | (SEQ ID NO: 400) | |
| HCC1954v2_CWSV01028 | 3 | GTACACTGAAGGCTCT | AGCAACAAAGATTATCC |
| | | ATGTGCAGGAACTCC | TAAGACTTCTGGAGGAA |
| | | (SEQ ID NO: 401) | G (SEQ ID NO: 50) |
| HCC1954v2_CWSV01070 | 3 | GTACCCTCCCAAAGTG | AGGAGGGAGTGACAGGA |
| | | CTGGGATTATAGGCA | TGTGATCTG (SEQ ID |
| | | (SEQ ID NO: 402) | NO: 52) |
| HCC1954v2_CWSV01075 | 3 | GTACAACTGTTCTTCCA | AGCCTGGCCAACACAGT |
| | | GGCCTGATTT (SEQ ID | GAAACC (SEQ ID NO: 54) |
| | | NO: 403) | |
| HCC1954v2_CWSV01077 | 3 | GTACCCCATCCTGGCA | TGACCTGTTCTTGGGTAG |
| | | CGTACTGCTACT (SEQ | CACTATTGGT (SEQ ID |
| | | ID NO: 404) | NO: 56) |
| HCC1954v2_CWSV01100 | 3 | GTACAGGCACACTCAG | AAAGGGATAGCAGTTTC |
| | | GCTTTACGATTCTTGTT | CTGCTGTCCATAG (SEQ |
| | | TA (SEQ ID NO: 405) | ID NO: 58) |
| HCC1954v2_CWSV01109 | 3 | GTACATTCTCCCTGCAA | CCCAGGCTGCTGGTCATG |
| | | ATTGTTTGACTCTAGAA | GAAA (SEQ ID NO: 60) |
| | | ATAGC (SEQ ID NO: 406) | |
| HCC1954v2_CWSV01130 | 3 | GTACGGGTAGTAGCGA | CACAGGGTTTATCTCCTC |
| | | TCTGGGTTACCCAG | AGACAAA (SEQ ID NO: 62) |
| | | (SEQ ID NO: 407) | |
| HCC1954v2_CWSV01177 | 3 | GTACCATTGACCCACC | GCGGACGCCATGGAGCT |
| | | GGTCATTCAGGAG (SEQ | (SEQ ID NO: 64) |
| | | ID NO: 408) | |
| HCC1954v2_CWSV01529 | 3 | GTACCTGAGGGTTATC | AACACATTAGTGATGTCT |
| | | CAAGTGACCCATTGCA | CAATGCCATCC (SEQ ID |
| | | (SEQ ID NO: 409) | NO: 66) |
| HCC1954v2_DNAIA_short1 | 3 | GTACCTTGTACCTGGTG | GTAGGCTGGGTAGCCAA |
| | | TTCGGTTAT (SEQ ID | ATC (SEQ ID NO: 2) |
| | | NO: 410) | |
| HCC1954v2_DNAIA_short2 | 3 | GTACCCAGCCCAAGAT | CTCTTCTTTCCTTTCAGC |
| | | GGATGAAT (SEQ ID | AACAC (SEQ ID NO: 4) |
| | | NO: 411) | |
| HCC1954v2_DNAIA_short3 | 3 | GTACGCGGTGGAAGTC | TCTCTCTCTCTCCAGTGA |
| | | ATTCTGATA (SEQ ID | ACAA (SEQ ID NO: 6) |
| | | NO: 412) | |
| HCC1954v2_DNAIA_short4 | 3 | GTACGAGGGCCACAGA | GATCACTTAGCTCAGGA |
| | | TTGGTATT (SEQ ID | AACAAAC (SEQ ID NO: 8) |
| | | NO: 413) | |
| HCC1954v2_DNAIA_short5 | 3 | GTACACTGAAAGGTCA | TACCGGTTTGTGTCCAAG |
| | | GATTCAGGAG (SEQ ID | AG (SEQ ID NO: 10) |
| | | NO: 414) | |
| HCC1954v2_CWSV00079 | 1 | ACGTTGAGGCTCGACA | GCCTGTCGTGCAGCCATG |
| | | GGTACCATATAGCCT | AGTTAG (SEQ ID NO: 12) |
| | | (SEQ ID NO: 415) | |
| HCC1954v2_CWSV00085 | 1 | ACGTGACTCCAACCAT | AAAGGAGAACTATGTGA |
| | | TGGTATAATCTGAAAG | GCTAATGCATTT (SEQ ID |
| | | TTGTCT (SEQ ID NO: 416) | NO: 14) |
| HCC1954v2_CWSV00096 | 1 | ACGTCTGCCTGACAAC | CTGTATATCTGGCTAGCC |
| | | GGACTGAAA (SEQ ID | ATATGCAGAAA (SEQ ID |
| | | NO: 417) | NO: 16) |
| HCC1954v2_CWSV00142 | 1 | ACGTTCGGCTATTTCTA | CAATCAGAAGACTGCTA |
| | | ACAGCTCACATGTTTAC | GGGTGGCTGTATT (SEQ |
| | | AT (SEQ ID NO: 418) | ID NO: 18) |
| HCC1954v2_CWSV00166 | 1 | ACGTTTCCATAAGTGCC | GAGTGTATAGACTAGAA |
| | | AGCTGGCTGAGAAATA | GTACAGCCATATAGTT |
| | | (SEQ ID NO: 419) | (SEQ ID NO: 20) |
| HCC1954v2_CWSV00203 | 1 | ACGTTCTGCTCCCAGGT | CCCTGTTGCCCAGGCTAG |
| | | TCAAGTGATTCTCAAG | AGTG (SEQ ID NO: 22) |
| | | (SEQ ID NO: 420) | |
| HCC1954v2_CWSV00281 | 1 | ACGTTCCGAGAGCTCA | GAGCGAGCTCAAGATAG |
| | | CAGCTTGGCT (SEQ ID | AGGCAGCTAA (SEQ ID |
| | | NO: 421) | NO: 24) |
| HCC1954v2_CWSV00291 | 1 | ACGTGTTTGATGGAATT | TCCACATCACGGGAGCA |
| | | CACCAGTGTGTTT (SEQ | GCTG (SEQ ID NO: 26) |
| | | ID NO: 422) | |
| HCC1954v2_CWSV00362 | 1 | ACGTATTTAGAGAGCC | GTCCAGCTGAGTGGAAC |
| | | TGTCACAAATGTCCAT | ATTGAAAGAACA (SEQ ID |
| | | ATGAGAA (SEQ ID | NO: 28) |
| | | NO: 423) | |
| HCC1954v2_CWSV00739 | 1 | ACGTGAGAGGAGAGAG | TAGGAGGAGCCCATTGG |
| | | TGCAGGGTCAA (SEQ ID | CC (SEQ ID NO: 30) |
| | | NO: 424) | |
| HCC1954v2_CWSV00742 | 1 | ACGTACATTATTGTTAA | GCAGGAGAATGGCATGA |
| | | CAAGGGTTGCTTCTGG | ACCCGG (SEQ ID NO: 32) |
| | | GAAGA (SEQ ID NO: 425) | |
| HCC1954v2_CWSV00743 | 1 | ACGTCCCTGGCACCTA | AGATGTGGCTGAACTTTA |
| | | GTGGGTCCATAC (SEQ | AATGCATATT (SEQ ID |
| | | ID NO: 426) | NO: 34) |
| HCC1954v2_CWSV00744 | 1 | ACGTGCACTCCAGAGA | AAAGGGCCAGGCCTGAG |
| | | CTCTGTTTCTAAA (SEQ | TGGT (SEQ ID NO: 36) |
| | | ID NO: 427) | |
| HCC1954v2_CWSV00791 | 1 | ACGTCAGGCATGGTAG | CTCCGGCACTGGGCAGA |
| | | CGCGCCT (SEQ ID | GGTA (SEQ ID NO: 38) |
| | | NO: 428) | |
| HCC1954v2_CWSV00841 | 1 | ACGTGGAAGCCTCATA | TCAGTGCTAGCTGATAAC |
| | | ATCATGGTGGAAGGCA | TCTGACTTT (SEQ ID |
| | | (SEQ ID NO: 429) | NO: 40) |
| HCC1954v2_CWSV00852 | 1 | ACGTCATGTTTACCGAT | CACTGCATGTTCCCAGTC |
| | | GAAGCTGCAAA (SEQ ID | ATAGGTGG (SEQ ID |
| | | NO: 430) | NO: 42) |
| HCC1954v2_CWSV00856 | 1 | ACGTAGGAAGCTCAGC | AGACCCATCTCACATGTA |
| | | TATACCTGTTACTGATA | GACACA (SEQ ID NO: 44) |
| | | ATTT (SEQ ID NO: 431) | |
| HCC1954v2_CWSV01009 | 1 | ACGTCCCATGCAGGCC | CTTCTCCAAGTCTCTACA |
| | | TAGGCTAGTGT (SEQ ID | CTTGGCCACAA (SEQ ID |
| | | NO: 432) | NO: 46) |
| HCC1954v2_CWSV01021 | 1 | ACGTACCTAAAGGACA | AGGATGACGCCTGGCTT |
| | | CATCTCAGTACATTT | GGAGAAT (SEQ ID NO: 48) |
| | | (SEQ ID NO: 433) | |
| HCC1954v2_CWSV01028 | 1 | ACGTACTGAAGGCTCT | AGCAACAAAGATTATCC |
| | | ATGTGCAGGAACTCC | TAAGACTTCTGGAGGAA |
| | | (SEQ ID NO: 434) | G (SEQ ID NO: 50) |
| HCC1954v2_CWSV01070 | 1 | ACGTCCTCCCAAAGTG | AGGAGGGAGTGACAGGA |
| | | CTGGGATTATAGGCA | TGTGATCTG (SEQ ID |
| | | (SEQ ID NO: 435) | NO: 52) |
| HCC1954v2_CWSV01075 | 1 | ACGTAACTGTTCTTCCA | AGCCTGGCCAACACAGT |
| | | GGCCTGATTT (SEQ ID | GAAACC (SEQ ID NO: 54) |
| | | NO: 436) | |
| HCC1954v2_CWSV01077 | 1 | ACGTCCCATCCTGGCA | TGACCTGTTCTTGGGTAG |
| | | CGTACTGCTACT (SEQ | CACTATTGGT (SEQ ID |
| | | ID NO: 437) | NO: 56) |
| HCC1954v2_CWSV01100 | 1 | ACGTAGGCACACTCAG | AAAGGGATAGCAGTTTC |
| | | GCTTTACGATTCTTGTT | CTGCTGTCCATAG (SEQ |
| | | TA (SEQ ID NO: 438) | ID NO: 58) |
| HCC1954v2_CWSV01109 | 1 | ACGTATTCTCCCTGCAA | CCCAGGCTGCTGGTCATG |
| | | ATTGTTTGACTCTAGAA | GAAA (SEQ ID NO: 60) |
| | | ATAGC (SEQ ID NO: 439) | |
| HCC1954v2_CWSV01130 | 1 | ACGTGGGTAGTAGCGA | CACAGGGTTTATCTCCTC |
| | | TCTGGGTTACCCAG | AGACAAA (SEQ ID NO: 62) |
| | | (SEQ ID NO: 440) | |
| HCC1954v2_CWSV01177 | 1 | ACGTCATTGACCCACC | GCGGACGCCATGGAGCT |
| | | GGTCATTCAGGAG (SEQ | (SEQ ID NO: 64) |
| | | ID NO: 441) | |
| HCC1954v2_CWSV01529 | 1 | ACGTCTGAGGGTTATC | AACACATTAGTGATGTCT |
| | | CAAGTGACCCATTGCA | CAATGCCATCC (SEQ ID |
| | | (SEQ ID NO: 442) | NO: 66) |
| HCC1954v2_DNAIA_short1 | 1 | ACGTCTTGTACCTGGTG | GTAGGCTGGGTAGCCAA |
| | | TTCGGTTAT (SEQ ID | ATC (SEQ ID NO: 2) |
| | | NO: 443) | |
| HCC1954v2_DNAIA_short2 | 1 | ACGTCCAGCCCAAGAT | CTCTTCTTTCCTTTCAGC |
| | | GGATGAAT (SEQ ID | AACAC (SEQ ID NO: 4) |
| | | NO: 444) | |
| HCC1954v2_DNAIA_short3 | 1 | ACGTGCGGTGGAAGTC | TCTCTCTCTCTCCAGTGA |
| | | ATTCTGATA (SEQ ID | ACAA (SEQ ID NO: 6) |
| | | NO: 445) | |
| HCC1954v2_DNAIA_short4 | 1 | ACGTGAGGGCCACAGA | GATCACTTAGCTCAGGA |
| | | TTGGTATT (SEQ ID | AACAAAC (SEQ ID NO: 8) |
| | | NO: 446) | |
| HCC1954v2_DNAIA_short5 | 1 | ACGTACTGAAAGGTCA | TACCGGTTTGTGTCCAAG |
| | | GATTCAGGAG (SEQ ID | AG (SEQ ID NO: 10) |
| | | NO: 447) | |

*Tag sequences are indicated with underline. SV targets and primers are indicated with “CWSV.” Control targets and primers are indicated with “DNAIA.”

### Alternative Approaches and Applications

One simple alternative approach to the tagged-primer design is to vary the length of primers on the 5′ end while keeping the 3′ end constant. Since our read out is sequencing-based, this can also guide the informatic approach to distinguish different versions of the same target panel.

The application of this approach is not restricted to SVEN. Any PCR-based assay that relies on detection of amplicons (mutated or otherwise) can benefit from this approach to track, measure, and exclude run-to-run carryover of PCR amplicons.

### REFERENCES

Aslanzadeh J. Preventing PCR amplification carryover contamination in a clinical laboratory. Ann Clin Lab Sci. 2004 Autumn; 34(4):389-96.
Garibyan L, Avashia N. Polymerase chain reaction. J Invest Dermatol. 2013 March; 133(3):1-4.
Mifflin T E. Setting up a PCR laboratory. CSH Protoc. 2007 Jul. 1; 2007:pdb.top14.

1. A method of analyzing nucleic acid with a group of primer sets, the method comprising sequentially amplifying template nucleic acid with each primer set within the group, wherein:
the group of primer sets comprises two or more primer sets;
each primer set comprises set-identity primer pairs;
each set-identity primer pair consists of set-identity primers consisting of a first set-identity primer and a second set-identity primer;
each set-identity primer comprises a template-annealing segment on a 3′ end of the set-identity primer;
each set-identity primer comprises a set-identity segment on a 5′ end of the set-identity primer;
a combination of the set-identity segment on the first set-identity primer and the set-identity segment on the second set-identity primer in each set-identity primer pair defines a set-identity signature; and
each of the set-identity signatures is defined only by set-identity primer pairs in one and only one of the primer sets within the group.

2. The method of claim 1, wherein each set-identity signature is defined by base length and base sequence of the set-identity segments on the first and second set-identity primers.

3. The method of claim 1, wherein each set-identity segment comprises from 0 to 10 bases.

4. The method of claim 1, wherein all the set-identity segments on all the second set-identity primers have a same base length and base sequence.

5. The method of claim 1, wherein each set-identity segment on each second set-identity primer has a base length of 0 bases.

6. The method of claim 1, wherein each set-identity segment on each first set-identity primer has a base length from 0 to 6 bases.

7. The method of claim 1, wherein all the set-identity segments on all the first set-identity primers have a same base length.

8. The method of claim 1, wherein each set-identity segment on each first set-identity primer within each primer set has a different base sequence than each set-identity segment on each first set-identity primer in each of the other primer sets.

9. The method of claim 1, wherein the set-identity segment on each first set-identity primer comprises a non-complimentary 5′ overhang.

10. The method of claim 1, wherein each primer set comprises at least 1 set-identity primer pair and up to 100 set-identity primer pairs, or at least 2 set-identity primer pairs and up to 100 set-identity primer pairs.

11. (canceled)

12. The method of claim 1, wherein:
the template-annealing segment of each of the first set-identity primers in each primer set has a base length and base sequence identical to a base length and base sequence of the template-annealing segment of at least one of the first set-identity primers in each of the other sets; and/or
the template-annealing segment of each of the second set-identity primers in each primer set has a base length and base sequence identical to a base length and base sequence of the template-annealing segment of at least one of the second set-identity primers in each of the other sets.

13. The method of claim 1, wherein each primer set comprises multiple copies of each first set-identity primer and each second set-identity primer.

14. The method of claim 1, wherein the all the set-identity primer pairs within each primer set together define no more than 10 different set-identity signatures.

15. The method of claim 1, wherein all the set-identity primer pairs within each primer set define the same set-identity signature.

16. The method of claim 1, wherein the group of primer sets comprises three or more primer sets.

17. The method of claim 1, wherein each sequential amplification comprises mixing the template nucleic acid and one of the primer sets, wherein the mixing for all the sequential amplifications is performed within a single enclosable mixing space.

18. The method of claim 1, further comprising:
generating a separate amplicon sample from each sequential amplification with each of the primer sets, wherein each amplicon sample comprises amplicons comprising one or more of the set-identity signatures; and
determining sequences of the amplicons in each separate amplicon sample, wherein the determining the sequences of the amplicons in each separate amplicon sample comprises determining the sequences of the amplicons in each separate amplicon sample in a separate workflow.

19. The method of claim 18, further comprising identifying the set-identity signatures in the sequences of the amplicons in each of the amplicon samples and informatically filtering the sequences of the amplicons in any given amplicon sample that comprise one of the set-identity signatures not defined by any of the set-identity primer pairs in the primer set employed in generating the given amplicon sample.

20. (canceled)

21. The method of claim 18, wherein the determining the sequences of the amplicons in each separate amplicon sample comprises generating a separate sequencing library from each separate amplicon sample, wherein the generating the separate sequencing libraries:
comprises sequentially generating each of the separate sequencing libraries;
is performed within a single enclosable library-generating space; and
comprises one or more steps selected from the group consisting of amplicon fragmentation, nucleic acid end repair, adapter ligation, and amplification with indexed or barcoded primers.

22-24. (canceled)

25. The method of claim 21, further comprising separately sequencing with a sequencing instrument each of the separate sequencing libraries, wherein the separately sequencing comprises sequentially sequencing with a sequencing instrument each of the sequencing libraries.

26. (canceled)
